(19) United States
(12) Patent Application Publication
(10) Pub. No.: US 2016/0228565 A1
(43) Pub. Date: Aug. 11, 2016

(54) POLYAMIDE BASED PEPTIDODENDRIMER CONJUGATES

(71) Applicant: New York University, New York, NY (US)

(72) Inventors: Marcus WECK, New York, NY (US); Stefania GALDIERO, Naples (IT); Rossella TARALLO, Astoria, NY (US); Thomas Patrick CARBERRY, III, Astoria, NY (US); Annarita FALANGA, Naples (IT); Massimiliano GALDIERO, Naples (IT)

(21) Appl. No.: 14/979,372

(22) Filed: Dec. 22, 2015

Related U.S. Application Data
(60) Provisional application No. 62/096,781, filed on Dec. 24, 2014.

(51) Int. Cl. A61K 47/48; C08G 69/48; C08G 69/28; A61K 38/16; C08G 69/26

(52) U.S. Cl. CPC A61K 47/48207; A61K 38/162; C08G 69/26; C08G 69/28; C08G 69/48

(57) ABSTRACT

The present invention relates to monofunctional and bifunctional peptidodendrimer conjugates that contain a polyamide dendrimer conjugated to Herpes Simplex Virus-1 glycoprotein-derived peptides. Also disclosed are pharmaceutical compositions containing these peptidodendrimer conjugates and methods of using these peptidodendrimer conjugates (e.g., to inhibit HSV-1 viral entry and to treat or prevent HSV-1 infection).

This application claims the benefit of U.S. Provisional Patent Application Ser. No. 62/096,781, filed Dec. 24, 2014, which is hereby incorporated by reference in its entirety.

### FIELD OF THE INVENTION

The present invention relates to peptidodendrimer conjugates for use against HSV-1.

### BACKGROUND OF THE INVENTION

Herpes simplex viruses (HSVs) are responsible for a wide variety of clinical manifestations and represent a significant worldwide disease and economic burden. There are two serotypes of HSV, HSV-1 and HSV-2, which primarily infect either oral or genital sites, respectively. For some populations, between 60% and 95% are infected with HSV-1 and between 6% and 50% with HSV-2 (van Benthem et al., Sex Transm. Infect. 77(2):120-24 (2001)). Even if HSV infections are often subclinical, their incidence and severity have increased over the past decades due to the increasing number of immunocompromised patients. In particular, the impact of genital herpes as a public health threat is amplified because of its epidemiological synergy with the human immunodeficiency virus (HIV) (Wald et al., J. Infect. Dis. 185:45-52 (2002)). Synthetic nucleoside analogs targeting viral DNA polymerase (e.g., acyclovir) are routinely used as standard treatment of symptomatic HSV infections (Superti et al., “New Advances in Anti-HSV Chemotherapy,” Curr. Med. Chem. 15(9):900-11 (2008)). However, their clinical use in immunocompromised patients receiving long-term treatments may lead to treatment failures due to the emergence of antiviral-resistant strains (Greco et al., “Novel Targets for the Development of Anti-Herpes Compounds,” Infect. Disord. Drug Targets 7(1):11-18 (2007)). Thus, it is imperative to develop new anti-HSV agents with antiviral activity based on alternative mechanisms of action. Inhibition of HSV attachment and/or entry represents a particularly attractive antiviral strategy since it may prevent the establishment of infection. Target compounds with this mode of action could provide a starting point for the development of topical microbicides that block transmission at the mucosal surface, thereby providing a method of prophylactic intervention (Keller et al., “Topical Microbicides for the Prevention of Genital Herpes Infection,” J. Antimicrob. Chemother. 55(4):420-23 (2005)).

Entry of enveloped viruses requires fusion of viral and cellular membranes, driven by conformational changes of viral glycoproteins. Herpes viruses are a paradigm for viral entry mediated by a multi-component fusion machinery. HSV enters host cells by fusion of the viral envelope with either the plasma membrane or an endosomal membrane, and the entry pathway is thought to be determined by both virus and host cell factors. In particular, HSV-1 enters cells through fusion of the viral envelope with a cellular membrane in a cascade of molecular interactions involving multiple viral glycoproteins and cellular receptors. The envelope glycoproteins gH/gL, gB, and gD are all essential for the entry process, and expression of this quartet of glycoproteins induces the fusion of cellular membranes in the absence of virus infection (Turner et al., J. Virol. 72:873-75 (1998)). Both gH/gL and gB constitute the core fusion machinery and cooperate to induce the initial lipid destabilization that ends in fusion.

In particular, initial interactions occur when viral envelope glycoprotein C (gC) binds to heparin sulfate on the cell surface. Glycoprotein D (gD), binds specifically to at least one of at least four known entry receptors (Akhtar & Shukla, “Viral Entry Mechanisms: Cellular and Viral Mediators of Herpes Simplex Virus Entry,” FEBS J. 276(24):7228-36 (2009)). These include herpes virus entry mediator (“HVEM”), nectin-1, nectin-2, and 3-O sulfated heparin sulfate. The receptor provides a strong, fixed attachment to the host cell. These interactions bring the membrane surfaces into mutual proximity and allow for other glycoproteins embedded in the viral envelope to interact with other cell surface molecules. Once bound to the HVEM, gD changes its conformation and interacts with viral glycoproteins H (gH) and L (gL), which form a complex. The interaction of these membrane proteins results in the hemifusion state. Afterward, gB interaction with the gH/gL complex creates an entry pore for the viral capsid. Glycoprotein B interacts with glycosaminoglycans on the surface of the host cell.

Numerous strategies have been traditionally pursued for the development of molecules with enhanced antiviral activities, such as nucleoside analogues, or modified natural products (Superti et al., Curr. Med. Chem. 15(9)900-11 (2008)). The use of peptides may allow the targeting of different steps of the virus replication cycle. Peptides may prevent viral attachment to host cell receptors (such as heparin sulfate) or inhibit the replication complex by interfering with protein-protein interactions, dissociating the complex and/or inhibiting its formation.

Peptides have several advantages: they can be highly specific and effective, they can be biodegraded by peptidases limiting their accumulation in tissues and resulting in lower toxicity, and they can exert a broad activity on different microorganisms.

The present invention is directed to overcoming these and other deficiencies in the art.

### SUMMARY OF THE INVENTION

One aspect of the present invention relates to a monofunctional peptidodendrimer conjugate comprising: a polyamide dendrimer conjugated with an HSV-1 envelope glycoprotein-derived peptide, wherein the peptide is a substituted or unsubstituted peptide selected from the group consisting of gB8, PgH, gC1, g1, and g2.

A second aspect of the present invention relates to a pharmaceutical composition comprising, in a pharmaceutically acceptable vehicle, (i) a first monofunctional peptidodendrimer conjugate comprising: a polyamide dendrimer conjugated with a first HSV-1 envelope glycoprotein-derived peptide; and (ii) a second monofunctional peptidodendrimer conjugate comprising: a polyamide dendrimer conjugated with a second HSV-1 envelope glycoprotein-derived peptide, wherein the first and second HSV-1 envelope glycoprotein-derived peptides are different.

A third aspect of the present invention relates to a bifunctional peptidodendrimer conjugate comprising: a polyamide dendrimer conjugated with two different HSV-1 envelope glycoprotein-derived peptides.

The present invention is further directed to pharmaceutical formulations containing the monofunctional peptidodendrimer conjugate and/or the bifunctional peptidodendrimer conjugate.

A fourth aspect of the present invention relates to a method of inhibiting entry of HSV-1 into a host cell. This method involves contacting the host cell, under conditions effective to inhibit entry of HSV-1 into the host cell, with:
(i) a monofunctional peptidodendrimer conjugate as described herein;
(ii) (a) a first monofunctional peptidodendrimer conjugate as described herein and (b) a second monofunctional peptidodendrimer conjugate as described herein, where the first and second monofunctional peptidodendrimer conjugates contain different HSV-1 glycoprotein-derived peptides;
(iii) a bifunctional peptidodendrimer conjugate as described herein; or
(iv) a combination thereof.

A fifth aspect of the present invention relates to a method of treating or preventing HSV-1 infection in a subject. This method involves administering to the subject, under conditions effective to treat or prevent HSV-1 infection:
(i) a monofunctional peptidodendrimer conjugate as described herein;
(ii) (a) a first monofunctional peptidodendrimer conjugate as described herein and (b) a second monofunctional peptidodendrimer conjugate as described herein, where the first and second monofunctional peptidodendrimer conjugates contain different HSV-1 glycoprotein-derived peptides;
(iii) a bifunctional peptidodendrimer conjugate as described herein; or
(iv) a combination thereof.

As demonstrated herein, peptidodendrimer conjugates containing monofunctional or bifunctional poly(amide)-based dendrimers functionalized with one or more peptides derived from HSV-1 envelope glycoproteins have the potential to inhibit HSV-1 infectivity.

### BRIEF DESCRIPTION OF THE DRAWINGS

FIGS. 1A-1C are the sequence alignment of gB glycoproteins sp|P06437|GB_HHV1K (SEQ ID NO:1), tr|Q9IWU4|Q9IWU4_HHV1 (SEQ ID NO:2), sp|P06436|GB_HHV1F (SEQ ID NO:3), tr|Q69076|Q69076_HHV1 (SEQ ID NO:4), sp|P10211|GB_HHV11 (SEQ ID NO:5), tr|Q69526|Q69526_HHV1 (SEQ ID NO:6), sp|P08665|GB_HHV1P (SEQ ID NO:7), and tr|Q9QLM8|Q9QLM8_HHV1 (SEQ ID NO:8), and the gB glycoprotein consensus sequence (SEQ ID NO:9).

FIG. 2 is the sequence alignment of gD glycoproteins sp|P06476|GD_HHV1H (SEQ ID NO:10), sp|Q69091|GD_HHV11 (SEQ ID NO:11), sp|P57083|GD_HHV1P (SEQ ID NO:12), and sp|P36318|GD_HHV1A (SEQ ID NO:13), and the gD glycoprotein consensus sequence (SEQ ID NO:14).

FIGS. 3A-3B are the sequence alignment of gH glycoproteins sp|P08356|GH_HHV1E (SEQ ID NO:15), sp|Q9DHD5|GH_HHV1F (SEQ ID NO:16), and sp|P06477|GH_HHV11 (SEQ ID NO:17), and the gH glycoprotein consensus sequence (SEQ ID NO:18).

FIGS. 4A-4D are analytical HPLC traces of Dendrimer-gB8 crude (FIG. 4A), Dendrimer-gB8 pure (FIG. 4B), gB503-523 control (FIG. 4C), and the dendrimer control (FIG. 4D).

FIG. 5 is the UV calibration curve of Pra-gB8 peptide at 280 nm.

FIG. 6 is a graph showing the results of the virus yield reduction assay described in Example 4. Each bar represents the inhibition percentage at the indicated concentrations of, from left to right, the dendrimer control, the gB8-dendrimer conjugate alone, the PgH-dendrimer conjugate alone, the 1:1 mixture of the gB8-dendrimer conjugate and the PgH-dendrimer conjugate, gB8 protein alone, and PgH protein alone.

FIG. 7 is a graph showing the results of the co-treatment assay described in Example 5. Each bar represents the inhibition percentage at the indicated concentrations of, from left to right, the dendrimer control, the gB8-dendrimer conjugate, the PgH-dendrimer conjugate, and the 1:1 mixture of the gB8 dendrimer conjugate and the PgH-dendrimer conjugate.

FIG. 8 is a graph showing the results of the virus pre-treatment assay described in Example 6. Each bar represents the inhibition percentage at the indicated concentrations of, from left to right, the dendrimer control, the gB8-dendrimer conjugate, the PgH-dendrimer conjugate, and the 1:1 mixture of the gB8 dendrimer conjugate and the PgH-dendrimer conjugate.

FIG. 9 is a graph showing the results of the cell pre-treatment assay described in Example 7. Each bar represents the inhibition percentage at the indicated concentrations of, from left to right, the dendrimer control, the gB8-dendrimer conjugate, the PgH-dendrimer conjugate, and the 1:1 mixture of the gB8 dendrimer conjugate and the PgH-dendrimer conjugate.

FIG. 10 is a graph showing the results of the post-treatment assay described in Example 8. Each bar represents the inhibition percentage at the indicated concentrations of, from left to right, the dendrimer control, the gB8-dendrimer conjugate, the PgH-dendrimer conjugate, and the 1:1 mixture of the gB8 dendrimer conjugate and the PgH-dendrimer conjugate.

FIGS. 11A-11B are the ¹H NMR (FIG. 11A) and ¹C NMR (FIG. 11B) spectra of dendron 8.

FIGS. 12A-12B are the ¹H NMR (FIG. 12A) and ¹C NMR (FIG. 12B) spectra of dendron 9.

FIGS. 13A-13B are the ¹H NMR (FIG. 13A) and ¹C NMR (FIG. 13B) spectra of dendron 10.

FIGS. 14A-14B are the ¹H NMR (FIG. 14A) and ¹C NMR (FIG. 14B) spectra of dendrimer 11.

FIGS. 15A-15B are the ¹H NMR (FIG. 15A) and ¹C NMR (FIG. 15B) spectra of dendrimer 12.

FIG. 16 is the ¹H NMR spectra of bifunctional dendrimer 13.

FIG. 17 is the mass spectra of bifunctional dendrimer 13. Matrix: 2,5-DHBA; dissolved in MeOH.

FIG. 18 is a graph showing the results of the virus yield reduction assay described in Example 11. Each bar represents the inhibition percentage at the indicated concentrations of, from left to right, the dendrimer control, the gB8-dendrimer conjugate alone, the PgH-dendrimer conjugate alone, the bifunctional gB8-PgH-dendrimer conjugate, gB8 protein alone, and PgH protein alone.

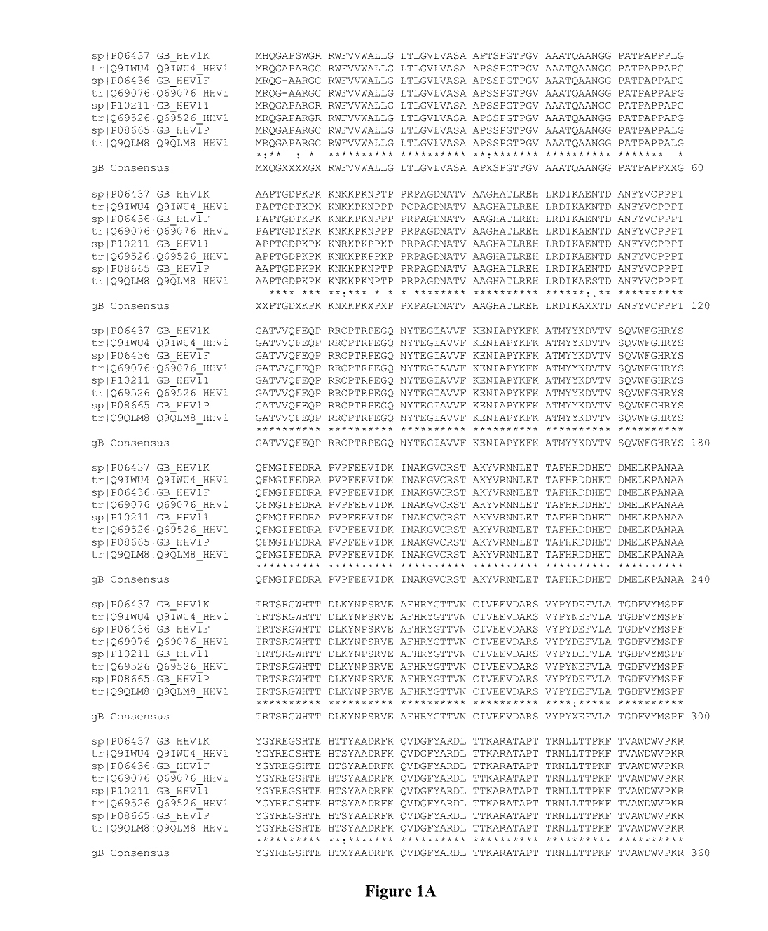

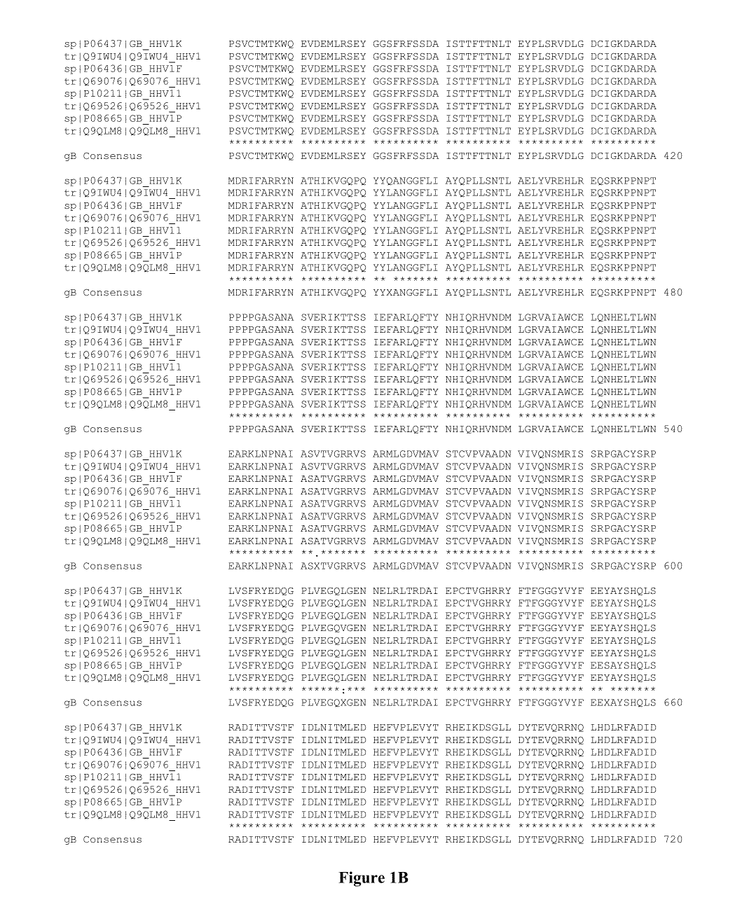

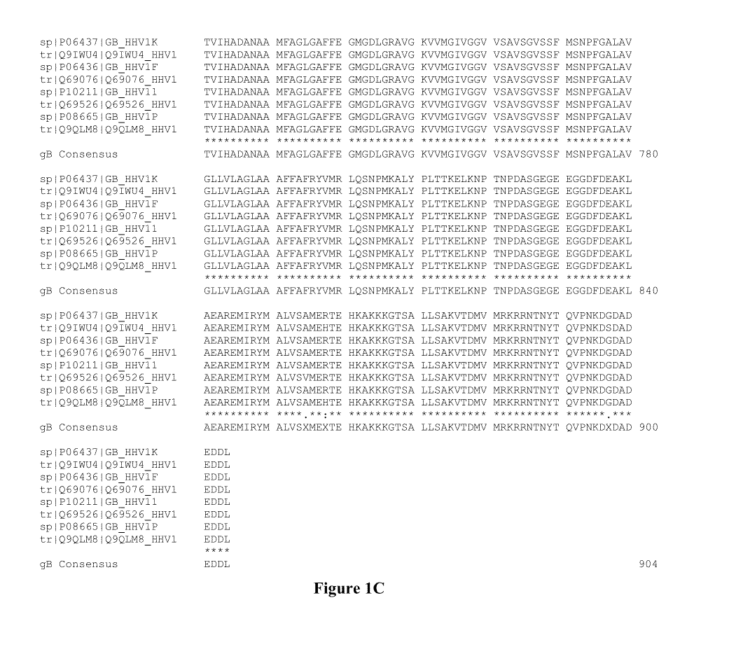

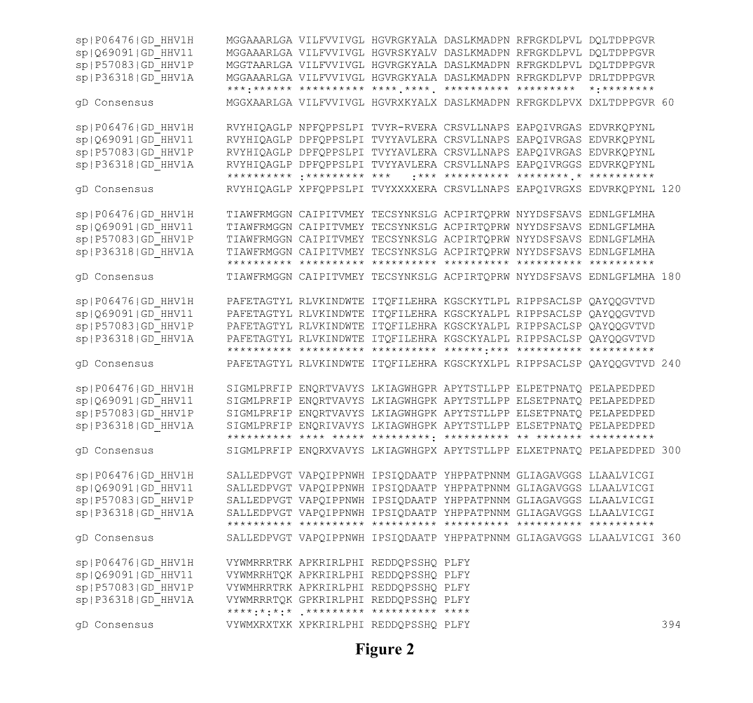

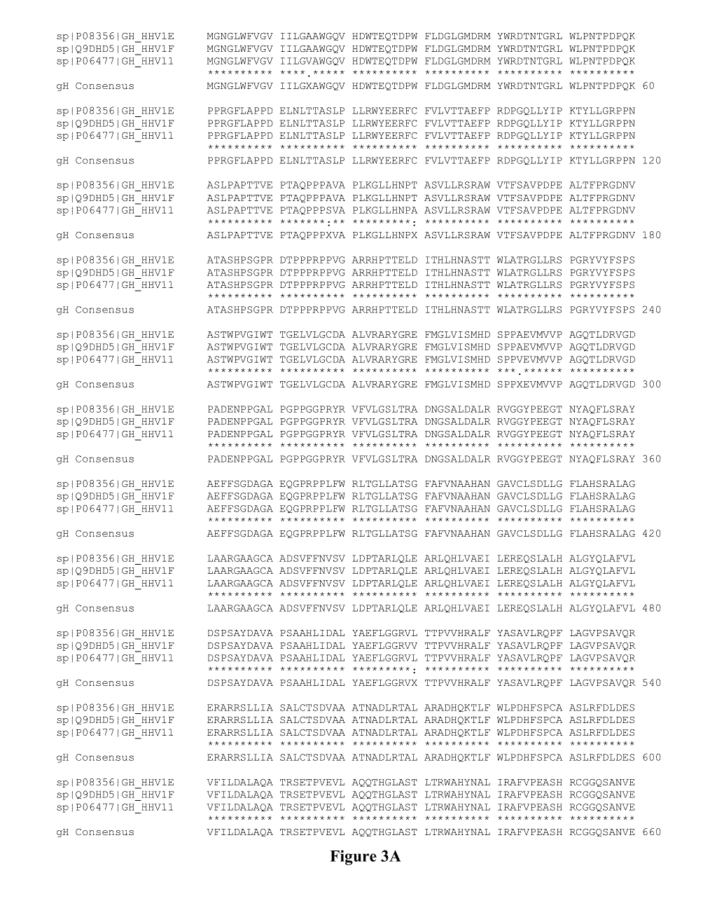

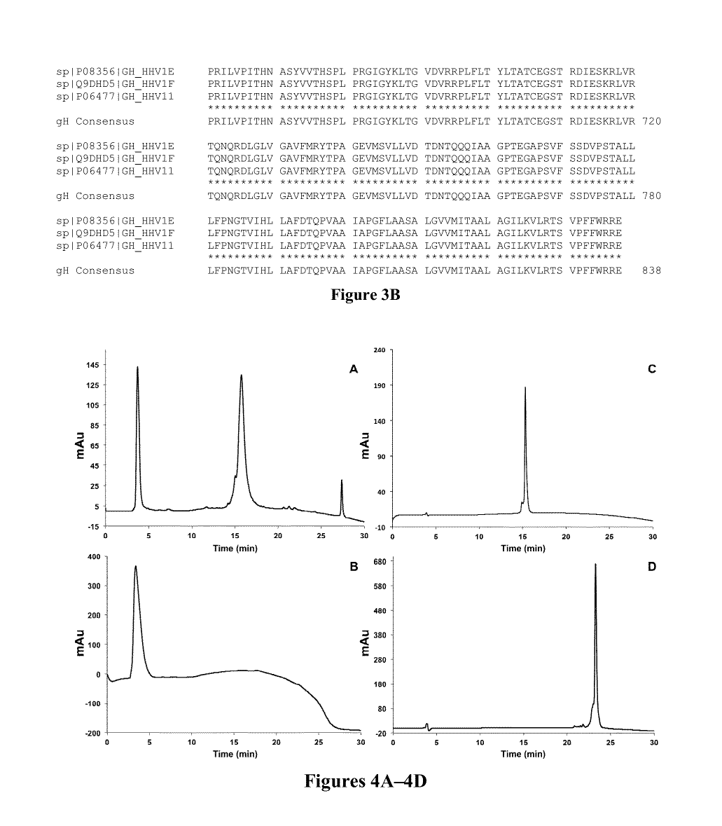

### DETAILED DESCRIPTION OF THE INVENTION

The present invention relates generally to monofunctional peptidodendrimer conjugates comprising: a polyamide dendrimer conjugated with an HSV-1 envelope glycoprotein-derived peptide; and to bifunctional peptidodendrimer conjugates comprising: a polyamide dendrimer conjugated with two different HSV-1 envelope glycoprotein-derived peptides.

Recent evidence suggests that helical domains as well as surface loops may play an important role in the fusion process and represent possible targets for therapeutic interference. In particular, helical sequences derived from gH and gB have shown the ability to inhibit HSV-1 infection of susceptible cells (Galdiero et al., J. Gen. Virol. 87(5):1085-97 (2006), which is hereby incorporated by reference in its entirety).

Different peptides may also show different inhibition pathways. The use of several peptides may help in interfering with different steps of the viral process. A combination of several gH and gB derived peptides and/or of peptides derived by gC could potentially give a compound which not only protects the cell from infection, but also kills unattached viruses. Furthermore, some sequences are also known to work cooperatively, so a system where the peptides are placed near one another could assist in those applications.

The monofunctional and bifunctional peptidodendrimers of the present invention each contain HSV-1 envelope glycoprotein-derived peptides. Unless stated otherwise, suitable HSV-1 envelope glycoproteins from which these peptides can be derived include gB, gC, gD, gH, and gL. Representative examples of these glycoproteins are shown in Table 1 below.

TABLE 1
Representative Glycoproteins

| | | UniProt | |
|---|---|---|---|
| | | Accession | |
| Glycoprotein | HSV Strain | No. | Sequence |
| gB | KOS | P06437 | MHQGAPSWGRRWFVVWALLGLTLGVLVASAAPTSPGTPG |
| | | | VAAATQAANGGPATPAPPPLGAAPTGDPKPKKNKKPKNPT |
| | | | PPRPAGDNATVAAGHATLREHLRDIKAENTDANFYVCPPPT |
| | | | GATVVQFEQPRRCPTRPEGQNYTEGIAVVFKENIAPYKFKAT |
| | | | MYYKDVTVSQVWFGHRYSQFMGIFEDRAPVPFEEVIDKINA |
| | | | KGVCRSTAKYVRNNLETTAFHRDDHETDMELKPANAATRTS |
| | | | RGWHTTDLKYNPSRVEAFHRYGTTVNCIVEEVDARSVYPYD |
| | | | EFVLATGDFVYMSPFYGYREGSHTEHTTYAADRFKQVDGFY |
| | | | ARDLTTKARATAPTTRNLLTTPKFTVAWDWVPKRPSVCTMT |
| | | | KWQEVDEMLRSEYGGSFRFSSDAISTTFTTNLTEYPLSRVDLG |
| | | | DCIGKDARDAMDRIFARRYNATHIKVGQPQYYQANGGFLIA |
| | | | YQPLLSNTLAELYVREHLREQSRKPPNPTPPPPGASANASVER |
| | | | IKTTSSIEFARLQFTYNHIQRHVNDMLGRVAIAWCELQNHEL |
| | | | TLWNEARKLNPNAIASVTVGRRVSARMLGDVMAVSTCVPV |
| | | | AADNVIVQNSMRISSRPGACYSRPLVSFRYEDQGPLVEGQLG |
| | | | ENNELRLTRDAIEPCTVGHRRYFTFGGGYVYFEEYAYSHQLSR |
| | | | ADITTVSTFIDLNITMLEDHEFVPLEVYTRHEIKDSGLLDYTEV |
| | | | QRRNQLHDLRFADIDTVIHADANAAMFAGLGAFFEGMGDL |
| | | | GRAVGKVVMGIVGGVVSAVSGVSSFMSNPFGALAVGLLVL |
| | | | AGLAAAFFAFRYVMRLQSNPMKALYPLTTKELKNPTNPDAS |
| | | | GEGEEGGDFDEAKLAEAREMIRYMALVSAMERTEHKAKKK |
| | | | GTSALLSAKVTDMVMRKRRNTNYTQVPNKDGDADEDDL |
| | | | (SEQ ID NO: 1) |
| gB | +GC | Q9IWU4 | MRQGAPARGCRWFVVWALLGLTLGVLVASAAPSSPGTPGV |
| | | | AAATQAANGGPATPAPPAPGPAPTGDTKPKKNKKPKNPPP |
| | | | PCPAGDNATVAAGHATLREHLRDIKAKNTDANFYVCPPPTG |
| | | | ATVVQFEQPRRCPTRPEGQNYTEGIAVVFKENIAPYKFKATM |
| | | | YYKDVTVSQVWFGHRYSQFMGIFEDRAPVPFEEVIDKINAK |
| | | | GVCRSTAKYVRNNLETTAFHRDDHETDMELKPANAATRTSR |
| | | | GWHTTDLKYNPSRVEAFHRYGTTVNCIVEEVDARSVYPYNEF |
| | | | VLATGDFVYMSPFYGYREGSHTEHTSYAADRFKQVDGFYAR |
| | | | DLTTKARATAPTTRNLLTTPKFTVAWDWVPKRPSVCTMTK |
| | | | WQEVDEMLRSEYGGSFRFSSDAISTTFTTNLTEYPLSRVDLG |
| | | | DCIGKDARDAMDRIFARRYNATHIKVGQPQYYLANGGFLIAY |
| | | | QPLLSNTLAELYVREHLREQSRKPPNPTPPPPGASANASVERI |
| | | | KTTSSIEFARLQFTYNHIQRHVNDMLGRVAIAWCELQNHELT |
| | | | LWNEARKLNPNAIASVTVGRRVSARMLGDVMAVSTCVPVA |
| | | | ADNVIVQNSMRISSRPGACYSRPLVSFRYEDQGPLVEGQLGE |
| | | | NNELRLTRDAIEPCTVGHRRYFTFGGGYVYFEEYAYSHQLSRA |
| | | | DITTVSTFIDLNITMLEDHEFVPLEVYTRHEIKDSGLLDYTEVQ |
| | | | RRNQLHDLRFADIDTVIHADANAAMFAGLGAFFEGMGDLG |
| | | | RAVGKVVMGIVGGVVSAVSGVSSFMSNPFGALAVGLLVLA |
| | | | GLAAAFFAFRYVMRLQSNPMKALYPLTTKELKNPTNPDASG |
| | | | EGEEGGDFDEAKLAEAREMIRYMALVSAMEHTEHKAKKKGT |
| | | | SALLSAKVTDMVMRKRRNTNYTQVPNKDSDADEDDL |
| | | | (SEQ ID NO: 2) |
| gB | F | P06436 | MRQGAARGCRWFVVWALLGLTLGVLVASAAPSSPGTPGVA |
| | | | AATQAANGGPATPAPPAPGPAPTGDTKPKKNKKPKNPPPP |
| | | | RPAGDNATVAAGHATLREHLRDIKAENTDANFYVCPPPTGA |
| | | | TVVQFEQPRRCPTRPEGQNYTEGIAVVFKENIAPYKFKATMY |
| | | | YKDVTVSQVWFGHRYSQFMGIFEDRAPVPFEEVIDKINAKG |
| | | | VCRSTAKYVRNNLETTAFHRDDHETDMELKPANAATRTSRG |
| | | | WHTTDLKYNPSRVEAFHRYGTTVNCIVEEVDARSVYPYDEFV |
| | | | LATGDFVYMSPFYGYREGSHTEHTSYAADRFKQVDGFYARD |
| | | | LTTKARATAPTTRNLLTTPKFTVAWDWVPKRPSVCTMTKW |
| | | | QEVDEMLRSEYGGSFRFSSDAISTTFTTNLTEYPLSRVDLGDCI |
| | | | GKDARDAMDRIFARRYNATHIKVGQPQYYLANGGFLIAYQP |
| | | | LLSNTLAELYVREHLREQSRKPPNPTPPPPGASANASVERIKT |
| | | | TSSIEFARLQFTYNHIQRHVNDMLGRVAIAWCELQNHELTL |
| | | | WNEARKLNPNAIASATVGRRVSARMLGDVMAVSTCVPVAA |
| | | | DNVIVQNSMRISSRPGACYSRPLVSFRYEDQGPLVEGQLGEN |
| | | | NELRLTRDAIEPCTVGHRRYFTFGGGYVYFEEYAYSHQLSRAD |
| | | | ITTVSTFIDLNITMLEDHEFVPLEVYTRHEIKDSGLLDYTEVQR |
| | | | RNQLHDLRFADIDTVIHADANAAMFAGLGAFFEGMGDLGR |
| | | | AVGKVVMGIVGGVVSAVSGVSSFMSNPFGALAVGLLVLAGL |
| | | | AAAFFAFRYVMRLQSNPMKALYPLTTKELKNPTNPDASGEG |
| | | | EEGGDFDEAKLAEAREMIRYMALVSAMERTEHKAKKKGTSA |
| | | | LLSAKVTDMVMRKRRNTNYTQVPNKDGDADEDDL |
| | | | (SEQ ID NO: 3) |
| gB | | Q69076 | MRQGAARGCRWFVVWALLGLTLGVLVASAAPSSPGTPGVA |
| | | | AATQAANGGPATPAPPAPGPAPTG DTKPKKNKKPKNPPPP |
| | | | RPAGDNATVAAGHATLREHLRDIKAENTDANFYVCPPPTGA |
| | | | TVVQFEQPRRCPTRPEGQNYTEGIAVVFKENIAPYKFKATMY |
| | | | YKDVTVSQVWFGHRYSQFMGIFEDRAPVPFEEVIDKINAKG |
| | | | VCRSTAKYVRNNLETTAFHRDDHETDMELKPANAATRTSRG |
| | | | WHTTDLKYNPSRVEAFHRYGTTVNCIVEEVDARSVYPYDEFV |
| | | | LATGDFVYMSPFYGYREGSHTEHTSYAADRFKQVDGFYARD |
| | | | LTTKARATAPTTRNLLTTPKFTVAWDWVPKRPSVCTMTKW |
| | | | QEVDEMLRSEYGGSFRFSSDAISTTFTTNLTEYPLSRVDLGDCI |
| | | | GKDARDAMDRIFARRYNATHIKVGQPQYYLANGGFLIAYQP |
| | | | LLSNTLAELYVREHLREQSRKPPNPTPPPPGASANASVERIKT |
| | | | TSSIEFARLQFTYNHIQRHVNDMLGRVAIAWCELQNHELTL |
| | | | WNEARKLNPNAIASATVGRRVSARMLGDVMAVSTCVPVAA |
| | | | DNVIVQNSMRISSRPGACYSRPLVSFRYEDQGPLVEGQVGE |
| | | | NNELRLTRDAIEPCTVGHRRYFTFGGGYVYFEEYAYSHQLSRA |
| | | | DITTVSTFIDLNITMLEDHEFVPLEVYTRHEIKDSGLLDYTEVQ |
| | | | RRNQLHDLRFADIDTVIHADANAAMFAGLGAFFEGMGDLG |
| | | | RAVGKVVMGIVGGVVSAVSGVSSFMSNPFGALAVGLLVLA |
| | | | GLAAAFFAFRYVMRLQSNPMKALYPLTTKELKNPTNPDASG |
| | | | EGEEGGDFDEAKLAEAREMIRYMALVSAMERTEHKAKKKGT |
| | | | SALLSAKVTDMVMRKRRNTNYTQVPNKDGDADEDDL |
| | | | (SEQ ID NO: 4) |
| gB | 17 | P10211 | MRQGAPARGRRWFVVWALLGLTLGVLVASAAPSSPGTPGV |
| | | | AAATQAANGGPATPAPPAPGAPPTGDPKPKKNRKPKPPKPP |
| | | | RPAGDNATVAAGHATLREHLRDIKAENTDANFYVCPPPTGA |
| | | | TVVQFEQPRRCPTRPEGQNYTEGIAVVFKENIAPYKFKATMY |
| | | | YKDVTVSQVWFGHRYSQFMGIFEDRAPVPFEEVIDKINAKG |
| | | | VCRSTAKYVRNNLETTAFHRDDHETDMELKPANAATRTSRG |
| | | | WHTTDLKYNPSRVEAFHRYGTTVNCIVEEVDARSVYPYDEFV |
| | | | LATGDFVYMSPFYGYREGSHTEHTSYAADRFKQVDGFYARD |
| | | | LTTKARATAPTTRNLLTTPKFTVAWDWVPKRPSVCTMTKW |
| | | | QEVDEMLRSEYGGSFRFSSDAISTTFTTNLTEYPLSRVDLGDCI |
| | | | GKDARDAMDRIFARRYNATHIKVGQPQYYLANGGFLIAYQP |
| | | | LLSNTLAELYVREHLREQSRKPPNPTPPPPGASANASVERIKT |
| | | | TSSIEFARLQFTYNHIQRHVNDMLGRVAIAWCELQNHELTL |
| | | | WNEARKLNPNAIASATVGRRVSARMLGDVMAVSTCVPVAA |
| | | | DNVIVQNSMRISSRPGACYSRPLVSFRYEDQGPLVEGQLGEN |
| | | | NELRLTRDAIEPCTVGHRRYFTFGGGYVYFEEYAYSHQLSRAD |
| | | | ITTVSTFIDLNITMLEDHEFVPLEVYTRHEIKDSGLLDYTEVQR |
| | | | RNQLHDLRFADIDTVIHADANAAMFAGLGAFFEGMGDLGR |
| | | | AVGKVVMGIVGGVVSAVSGVSSFMSNPFGALAVGLLVLAGL |
| | | | AAAFFAFRYVMRLQSNPMKALYPLTTKELKNPTNPDASGEG |
| | | | EEGGDFDEAKLAEAREMIRYMALVSAMERTEHKAKKKGTSA |
| | | | LLSAKVTDMVMRKRRNTNYTQVPNKDGDADEDDL |
| | | | (SEQ ID NO: 5) |
| gB | AGNpath | Q69526 | MRQGAPARGRRWFVVWALLGLTLGVLVASAAPSSPGTPGV |
| | | | AAATQAANGGPATPAPPAPGAPPTGDPKPKKNKKPKPPKPP |
| | | | RPAGDNATVAAGHATLREHLRDIKAENTDANFYVCPPPTGA |
| | | | TVVQFEQPRRCPTRPEGQNYTEGIAVVFKENIAPYKFKATMY |
| | | | YKDVTVSQVWFGHRYSQFMGIFEDRAPVPFEEVIDKINAKG |
| | | | VCRSTAKYVRNNLETTAFHRDDHETDMELKPANAATRTSRG |
| | | | WHTTDLKYNPSRVEAFHRYGTTVNCIVEEVDARSVYPYNEFV |
| | | | LATGDFVYMSPFYGYREGSHTEHTSYAADRFKQVDGFYARD |
| | | | LTTKARATAPTTRNLLTTPKFTVAWDWVPKRPSVCTMTKW |
| | | | QEVDEMLRSEYGGSFRFSSDAISTTFTTNLTEYPLSRVDLGDCI |
| | | | GKDARDAMDRIFARRYNATHIKVGQPQYYLANGGFLIAYQP |
| | | | LLSNTLAELYVREHLREQSRKPPNPTPPPPGASANASVERIKT |
| | | | TSSIEFARLQFTYNHIQRHVNDMLGRVAIAWCELQNHELTL |
| | | | WNEARKLNPNAIASATVGRRVSARMLGDVMAVSTCVPVAA |
| | | | DNVIVQNSMRISSRPGACYSRPLVSFRYEDQGPLVEGQLGEN |
| | | | NELRLTRDAIEPCTVGHRRYFTFGGGYVYFEEYAYSHQLSRAD |
| | | | ITTVSTFIDLNITMLEDHEFVPLEVYTRHEIKDSGLLDYTEVQR |
| | | | RNQLHDLRFADIDTVIHADANAAMFAGLGAFFEGMGDLGR |
| | | | AVGKVVMGIVGGVVSAVSGVSSFMSNPFGALAVGLLVLAGL |
| | | | AAAFFAFRYVMRLQSNPMKALYPLTTKELKNPTNPDASGEG |
| | | | EEGGDFDEAKLAEAREMIRYMALVSVMERTEHKAKKKGTSA |
| | | | LLSAKVTDMVMRKRRNTNYTQVPNKDGDADEDDL |
| | | | (SEQ ID NO: 6) |
| gB | Patton | P08665 | MRQGAPARGCRWFVVWALLGLTLGVLVASAAPSSPGTPGV |
| | | | AAATQAANGGPATPAPPALGAAPTGDPKPKKNKKPKNPTP |
| | | | PRPAGDNATVAAGHATLREHLRDIKAENTDANFYVCPPPTG |
| | | | ATVVQFEQPRRCPTRPEGQNYTEGIAVVFKENIAPYKFKATM |
| | | | YYKDVTVSQVWFGHRYSQFMGIFEDRAPVPFEEVIDKINAK |
| | | | GVCRSTAKYVRNNLETTAFHRDDHETDMELKPANAATRTSR |
| | | | GWHTTDLKYNPSRVEAFHRYGTTVNCIVEEVDARSVYPYDEF |
| | | | VLATGDFVYMSPFYGYREGSHTEHTSYAADRFKQVDGFYAR |
| | | | DLTTKARATAPTTRNLLTTPKFTVAWDWVPKRPSVCTMTK |
| | | | WQEVDEMLRSEYGGSFRFSSDAISTTFTTNLTEYPLSRVDLG |
| | | | DCIGKDARDAMDRIFARRYNATHIKVGQPQYYLANGGFLIAY |
| | | | QPLLSNTLAELYVREHLREQSRKPPNPTPPPPGASANASVERI |
| | | | KTTSSIEFARLQFTYNHIQRHVNDMLGRVAIAWCELQNHELT |
| | | | LWNEARKLNPNAIASATVGRRVSARMLGDVMAVSTCVPVA |
| | | | ADNVIVQNSMRISSRPGACYSRPLVSFRYEDQGPLVEGQLGE |
| | | | NNELRLTRDAIEPCTVGHRRYFTFGGGYVYFEESAYSHQLSRA |
| | | | DITTVSTFIDLNITMLEDHEFVPLEVYTRHEIKDSGLLDYTEVQ |
| | | | RRNQLHDLRFADIDTVIHADANAAMFAGLGAFFEGMGDLG |
| | | | RAVGKVVMGIVGGVVSAVSGVSSFMSNPFGALAVGLLVLA |
| | | | GLAAAFFAFRYVMRLQSNPMKALYPLTTKELKNPTNPDASG |
| | | | EGEEGGDFDEAKLAEAREMIRYMALVSAMERTEHKAKKKGT |
| | | | SALLSAKVTDMVM RKRRNTNYTQVPNKDGDADEDDL |
| | | | (SEQ ID NO: 7) |
| gB | HSZP | Q9QLM8 | MRQGAPARGCRWFVVWALLGLTLGVLVASAAPSSPGTPGV |
| | | | AAATQAANGGPATPAPPALGAAPTGDPKPKKNKKPKNPTP |
| | | | PRPAGDNATVAAGHATLREHLRDIKAESTDANFYVCPPPTG |
| | | | ATVVQFEQPRRCPTRPEGQNYTEGIAVVFKENIAPYKFKATM |
| | | | YYKDVTVSQVWFGHRYSQFMGIFEDRAPVPFEEVIDKINAK |
| | | | GVCRSTAKYVRNNLETTAFHRDDHETDMELKPANAATRTSR |
| | | | GWHTTDLKYNPSRVEAFHRYGTTVNCIVEEVDARSVYPYDEF |
| | | | VLATGDFVYMSPFYGYREGSHTEHTSYAADRFKQVDGFYAR |
| | | | DLTTKARATAPTTRNLLTTPKFTVAWDWVPKRPSVCTMTK |
| | | | WQEVDEMLRSEYGGSFRFSSDAISTTFTTNLTEYPLSRVDLG |
| | | | DCIGKDARDAMDRIFARRYNATHIKVGQPQYYLANGGFLIAY |
| | | | QPLLSNTLAELYVREHLREQSRKPPNPTPPPPGASANASVERI |
| | | | KTTSSIEFARLQFTYNHIQRHVNDMLGRVAIAWCELQNHELT |
| | | | LWNEARKLNPNAIASATVGRRVSARMLGDVMAVSTCVPVA |
| | | | ADNVIVQNSMRISSRPGACYSRPLVSFRYEDQGPLVEGQLGE |
| | | | NNELRLTRDAIEPCTVGHRRYFTFGGGYVYFEEYAYSHQLSRA |
| | | | DITTVSTFIDLNITMLEDHEFVPLEVYTRHEIKDSGLLDYTEVQ |
| | | | RRNQLHDLRFADIDTVIHADANAAMFAGLGAFFEGMGDLG |
| | | | RAVGKVVMGIVGGVVSAVSGVSSFMSNPFGALAVGLLVLA |
| | | | GLAAAFFAFRYVMRLQSNPMKALYPLTTKELKNPTNPDASG |
| | | | EGEEGGDFDEAKLAEAREMIRYMALVSAMEHTEHKAKKKGT |
| | | | SALLSAKVTDMVMRKRRNTNYTQVPNKDGDADEDDL |
| | | | (SEQ ID NO: 8) |
| gB | Consensus | N/A | MXQGXXXXGXRWFVVWALLGLTLGVLVASAAPXSPGTPGV |
| | Sequence | | AAATQAANGGPATPAPPXXGXXPTGDXKPKKNXKPKXPXPP |
| | (see FIGS. | | XPAGDNATVAAGHATLREHLRDIKAXXTDANFYVCPPPTGA |
| | 1A-1C) | | TVVQFEQPRRCPTRPEGQNYTEGIAVVFKENIAPYKFKATMY |
| | | | YKDVTVSQVWFGHRYSQFMGIFEDRAPVPFEEVIDKINAKG |
| | | | VCRSTAKYVRNNLETTAFHRDDHETDMELKPANAATRTSRG |
| | | | WHTTDLKYNPSRVEAFHRYGTTVNCIVEEVDARSVYPYXEFV |
| | | | LATGDFVYMSPFYGYREGSHTEHTXYAADRFKQVDGFYARD |
| | | | LTTKARATAPTTRNLLTTPKFTVAWDWVPKRPSVCTMTKW |
| | | | QEVDEMLRSEYGGSFRFSSDAISTTFTTNLTEYPLSRVDLGDCI |
| | | | GKDARDAMDRIFARRYNATHIKVGQPQYYXANGGFLIAYQP |
| | | | LLSNTLAELYVREHLREQSRKPPNPTPPPPGASANASVERIKT |
| | | | TSSIEFARLQFTYNHIQRHVNDMLGRVAIAWCELQNHELTL |
| | | | WNEARKLNPNAIASXTVGRRVSARMLGDVMAVSTCVPVAA |
| | | | DNVIVQNSMRISSRPGACYSRPLVSFRYEDQGPLVEGQXGE |
| | | | NNELRLTRDAIEPCTVGHRRYFTFGGGYVYFEEXAYSHQLSR |
| | | | ADITTVSTFIDLNITMLEDHEFVPLEVYTRHEIKDSGLLDYTEV |
| | | | QRRNQLHDLRFADIDTVIHADANAAMFAGLGAFFEGMGDL |
| | | | GRAVGKVVMGIVGGVVSAVSGVSSFMSNPFGALAVGLLVL |
| | | | AGLAAAFFAFRYVMRLQSNPMKALYPLTTKELKNPTNPDAS |
| | | | GEGEEGGDFDEAKLAEAREMIRYMALVSXMEXTEHKAKKKG |
| | | | TSALLSAKVTDMVMRKRRNTNYTQVPNKDXDADEDDL |
| | | | (SEQ ID NO: 9) |
| | | | (X at residue 2 is H or R; X at residue 5 is absent or A; X |
| | | | at residue 6 is P or A; X at residue 7 is S or A; X at |
| | | | residue 8 is W or R; X at residue 10 is R or C; X at |
| | | | residue 33 is T or 5; X at residue 58 is P or A; X at |
| | | | residue 59 is L or P; X at residue 61 is A or P; X at |
| | | | residue 62 is A or P; X at residue 67 is P or T; X at |
| | | | residue 73 is K or R; X at residue 77 is N or P; X at |
| | | | residue 79 is T, P, or K; X at residue 82 is R or C; X at |
| | | | residue 107 is E or K; X at residue 108 is N or S; X at |
| | | | residue 285 is D or N; X at residue 313 is T or S; X at |
| | | | residue 443 is Q or L; X at residue 553 is V or A; X at |
| | | | residue 617 is L or V; X at residue 653 is Y or S; X at |
| | | | residue 855 is A or V; X at residue 858 is R or H; X at |
| | | | residue 897 is G or S) |
| gC | KOS | P28986 | MAPGRVGLAVVLWGLLWLGAGVAGGSETASTGPTITAGAV |
| | | | TNASEAPTSGSPGSAASPEVTPTSTPNPNNVTQNKTTPTEPA |
| | | | SPPTTPKPTSTPKSPPTSTPDPKPKNNTTPAKSGRPTKPPGPV |
| | | | WCDRRDPLARYGSRVQIRCRFRNSTRMEFRLQIWRYSMGP |
| | | | SPPIAPAPDLEEVLTNITAPPGGLLVYDSAPNLTDPHVLWAEG |
| | | | AGPGADPPLYSVTGPLPTQRLIIGEVTPATQGMYYLAWGRM |
| | | | DSPHEYGTWVRVRMFRPPSLTLQPHAVMEGQPFKATCTAA |
| | | | AYYPRNPVEFDWFEDDRQVFNPGQIDTQTHEHPDGFTTVST |
| | | | VTSEAVGGQVPPRTFTCQMTWHRDSVTFSRRNATGLALVLP |
| | | | RPTITMEFGVRHVVCTAGCVPEGVTFAWFLGDDPSPAAKSA |
| | | | VTAQESCDHPGLATVRSTLPISYDYSEYICRLTGYPAGIPVLEH |
| | | | HGSHQPPPRDPTERQVIEAIEWVGIGIGVLAAGVLVVTAIVY |
| | | | VVRTSQSRQRHRR (SEQ ID NO: 19) |
| gD | HZT | P06476 | MGGAAARLGAVILFVVIVGLHGVRGKYALADASLKMADPNR |
| | | | FRGKDLPVLDQLTDPPGVRRVYHIQAGLPNPFQPPSLPITVYR |
| | | | RVERACRSVLLNAPSEAPQIVRGASEDVRKQPYNLTIAWFR |
| | | | MGGNCAIPITVMEYTECSYNKSLGACPIRTQPRWNYYDSFSA |
| | | | VSEDNLGFLMHAPAFETAGTYLRLVKINDWTEITQFILEHRAK |
| | | | GSCKYTLPLRIPPSACLSPQAYQQGVTVDSIGMLPRFIPENQR |
| | | | TVAVYSLKIAGWHGPRAPYTSTLLPPELPETPNATQPELAPED |
| | | | PEDSALLEDPVGTVAPQIPPNWHIPSIQDAATPYHPPATPNN |
| | | | MGLIAGAVGGSLLAALVICGIVYWMRRRTRKAPKRIRLPHIRE |
| | | | DDQPSSHQPLFY (SEQ ID NO: 10) |
| gD | 17 | Q69091 | MGGAAARLGAVILFVVIVGLHGVRSKYALVDASLKMADPNR |
| | | | FRGKDLPVLDQLTDPPGVRRVYHIQAGLPDPFQPPSLPITVYY |
| | | | AVLERACRSVLLNAPSEAPQIVRGASEDVRKQPYNLTIAWFR |
| | | | MGGNCAIPITVMEYTECSYNKSLGACPIRTQPRWNYYDSFSA |
| | | | VSEDNLGFLMHAPAFETAGTYLRLVKINDWTEITQFILEHRAK |
| | | | GSCKYALPLRIPPSACLSPQAYQQGVTVDSIGMLPRFIPENQR |
| | | | TVAVYSLKIAGWHGPKAPYTSTLLPPELSETPNATQPELAPED |
| | | | PEDSALLEDPVGTVAPQIPPNWHIPSIQDAATPYHPPATPNN |
| | | | MGLIAGAVGGSLLAALVICGIVYWMRRHTQKAPKRIRLPHIR |
| | | | EDDQPSSHQPLFY (SEQ ID NO: 11) |
| gD | Patton | P57083 | MGGTAARLGAVILFVVIVGLHGVRGKYALADASLKMADPNR |
| | | | FRGKDLPVLDQLTDPPGVRRVYHIQAGLPDPFQPPSLPITVYY |
| | | | AVLERACRSVLLNAPSEAPQIVRGASEDVRKQPYNLTIAWFR |
| | | | MGGNCAIPITVMEYTECSYNKSLGACPIRTQPRWNYYDSFSA |
| | | | VSEDNLGFLMHAPAFETAGTYLRLVKINDWTEITQFILEHRAK |
| | | | GSCKYALPLRIPPSACLSPQAYQQGVTVDSIGMLPRFIPENQR |
| | | | TVAVYSLKIAGWHGPKAPYTSTLLPPELSETPNATQPELAPED |
| | | | PEDSALLEDPVGTVAPQIPPNWHIPSIQDAATPYHPPATPNN |
| | | | MGLIAGAVGGSLLAALVICGIVYWMHRRTRKAPKRIRLPHIR |
| | | | EDDQPSSHQPLFY (SEQ ID NO: 12) |
| gD | Angelotti | P36318 | MGGAAARLGAVILFVVIVGLHGVRGKYALADASLKMADPNR |
| | | | FRGKDLPVPDRLTDPPGVRRVYHIQAGLPDPFQPPSLPITVYY |
| | | | AVLERACRSVLLNAPSEAPQIVRGGSEDVRKQPYNLTIAWFR |
| | | | MGGNCAIPITVMEYTECSYNKSLGACPIRTQPRWNYYDSFSA |
| | | | VSEDNLGFLMHAPAFETAGTYLRLVKINDWTEITQFILEHRAK |
| | | | GSCKYALPLRIPPSACLSPQAYQQGVTVDSIGMLPRFIPENQR |
| | | | IVAVYSLKIAGWHGPKAPYTSTLLPPELSETPNATQPELAPED |
| | | | PEDSALLEDPVGTVAPQIPPNWHIPSIQDAATPYHPPATPNN |
| | | | MGLIAGAVGGSLLAALVICGIVYWMRRRTQKGPKRIRLPHIR |
| | | | EDDQPSSHQPLFY (SEQ ID NO: 13) |
| gD | Consensus | N/A | MGGXAARLGAVILFVVIVGLHGVRXKYALXDASLKMADPNR |
| | Sequence | | FRGKDLPVXDXLTDPPGVRRVYHIQAGLPXPFQPPSLPITVYX |
| | (see FIG. | | XXXERACRSVLLNAPSEAPQIVRGXSEDVRKQPYNLTIAWFR |
| | 2) | | MGGNCAIPITVMEYTECSYNKSLGACPIRTQPRWNYYDSFSA |
| | | | VSEDNLGFLMHAPAFETAGTYLRLVKINDWTEITQFILEHRAK |
| | | | GSCKYXLPLRIPPSACLSPQAYQQGVTVDSIGMLPRFIPENQR |
| | | | XVAVYSLKIAGWHGPXAPYTSTLLPPELXETPNATQPELAPED |
| | | | PEDSALLEDPVGTVAPQIPPNWHIPSIQDAATPYHPPATPNN |
| | | | MGLIAGAVGGSLLAALVICGIVYWMXRXTXKXPKRIRLPHIRE |
| | | | DDQPSSHQPLFY (SEQ ID NO: 14) |
| | | | (X at residue 4 is A or T; X at residue 25 is G or 5; X at |
| | | | residue 30 is A or V; X at residue 50 is L or P; X at |
| | | | residue 52 is Q or R; X at residue 71 is N or D; X at |
| | | | residue 84 is R or Y; X at residue 85 is absent or A; X at |
| | | | residue 86 is R or V; X at residue 87 is V or L; X at |
| | | | residue 109 is A or G; X at residue 217 is T or A; X at |
| | | | residue 255 is T or I; X at residue 270 is R or K; X at |
| | | | residue 283 is P or 5; X at residue 365 is R or H; X at |
| | | | residue 367 is R or H; X at residue 369 is R or Q; X at |
| | | | residue 371 is A or G) |
| gH | HFEM | P08356 | MGNGLWFVGVIILGAAWGQVHDWTEQTDPWFLDGLGM |
| | | | DRMYWRDTNTGRLWLPNTPDPQKPPRGFLAPPDELNLTTA |
| | | | SLPLLRWYEERFCFVLVTTAEFPRDPGQLLYIPKTYLLGRPPNA |
| | | | SLPAPTTVEPTAQPPPAVAPLKGLLHNPTASVLLRSRAWVTF |
| | | | SAVPDPEALTFPRGDNVATASHPSGPRDTPPPRPPVGARRH |
| | | | PTTELDITHLHNASTTWLATRGLLRSPGRYVYFSPSASTWPV |
| | | | GIWTTGELVLGCDAALVRARYGREFMGLVISMHDSPPAEV |
| | | | MVVPAGQTLDRVGDPADENPPGALPGPPGGPRYRVFVLGS |
| | | | LTRADNGSALDALRRVGGYPEEGTNYAQFLSRAYAEFFSGDA |
| | | | GAEQGPRPPLFWRLTGLLATSGFAFVNAAHANGAVCLSDLL |
| | | | GFLAHSRALAGLAARGAAGCAADSVFFNVSVLDPTARLQLE |
| | | | ARLQHLVAEILEREQSLALHALGYQLAFVLDSPSAYDAVAPSA |
| | | | AHLIDALYAEFLGGRVLTTPVVHRALFYASAVLRQPFLAGVPS |
| | | | AVQRERARRSLLIASALCTSDVAAATNADLRTALARADHQKT |
| | | | LFWLPDHFSPCAASLRFDLDESVFILDALAQATRSETPVEVLA |
| | | | QQTHGLASTLTRWAHYNALIRAFVPEASHRCGGQSANVEPR |
| | | | ILVPITHNASYVVTHSPLPRGIGYKLTGVDVRRPLFLTYLTATC |
| | | | EGSTRDIESKRLVRTQNQRDLGLVGAVFMRYTPAGEVMSVL |
| | | | LVDTDNTQQQIAAGPTEGAPSVFSSDVPSTALLLFPNGTVIHL |
| | | | LAFDTQPVAAIAPGFLAASALGVVMITAALAGILKVLRTSVPF |
| | | | FWRRE (SEQ ID NO: 15) |
| gH | F | Q9DHD5 | MGNGLWFVGVIILGAAWGQVHDWTEQTDPWFLDGLGM |
| | | | DRMYWRDTNTGRLWLPNTPDPQKPPRGFLAPPDELNLTTA |
| | | | SLPLLRWYEERFCFVLVTTAEFPRDPGQLLYIPKTYLLGRPPNA |
| | | | SLPAPTTVEPTAQPPPAVAPLKGLLHNPTASVLLRSRAWVTF |
| | | | SAVPDPEALTFPRGDNVATASHPSGPRDTPPPRPPVGARRH |
| | | | PTTELDITHLHNASTTWLATRGLLRSPGRYVYFSPSASTWPV |
| | | | GIWTTGELVLGCDAALVRARYGREFMGLVISMHDSPPAEV |
| | | | MVVPAGQTLDRVGDPADENPPGALPGPPGGPRYRVFVLGS |
| | | | LTRADNGSALDALRRVGGYPEEGTNYAQFLSRAYAEFFSGDA |
| | | | GAEQGPRPPLFWRLTGLLATSGFAFVNAAHANGAVCLSDLL |
| | | | GFLAHSRALAGLAARGAAGCAADSVFFNVSVLDPTARLQLE |
| | | | ARLQHLVAEILEREQSLALHALGYQLAFVLDSPSAYDAVAPSA |
| | | | AHLIDALYAEFLGGRVVTTPVVHRALFYASAVLRQPFLAGVPS |
| | | | AVQRERARRSLLIASALCTSDVAAATNADLRTALARADHQKT |
| | | | LFWLPDHFSPCAASLRFDLDESVFILDALAQATRSETPVEVLA |
| | | | QQTHGLASTLTRWAHYNALIRAFVPEASHRCGGQSANVEPR |
| | | | ILVPITHNASYVVTHSPLPRGIGYKLTGVDVRRPLFLTYLTATC |
| | | | EGSTRDIESKRLVRTQNQRDLGLVGAVFMRYTPAGEVMSVL |
| | | | LVDTDNTQQQIAAGPTEGAPSVFSSDVPSTALLLFPNGTVIHL |
| | | | LAFDTQPVAAIAPGFLAASALGVVMITAALAGILKVLRTSVPF |
| | | | FWRRE (SEQ ID NO: 16) |
| gH | 17 | P06477 | MGNGLWFVGVIILGVAWGQVHDWTEQTDPWFLDGLGM |
| | | | DRMYWRDTNTGRLWLPNTPDPQKPPRGFLAPPDELNLTTA |
| | | | SLPLLRWYEERFCFVLVTTAEFPRDPGQLLYIPKTYLLGRPPNA |
| | | | SLPAPTTVEPTAQPPPSVAPLKGLLHNPAASVLLRSRAWVTFS |
| | | | AVPDPEALTFPRGDNVATASHPSGPRDTPPPRPPVGARRHP |
| | | | TTELDITHLHNASTTWLATRGLLRSPGRYVYFSPSASTWPVGI |
| | | | VVTTGELVLGCDAALVRARYGREFMGLVISMHDSPPVEVMV |
| | | | VPAGQTLDRVGDPADENPPGALPGPPGGPRYRVFVLGSLTR |
| | | | ADNGSALDALRRVGGYPEEGTNYAQFLSRAYAEFFSGDAGA |
| | | | EQGPRPPLFWRLTGLLATSGFAFVNAAHANGAVCLSDLLGFL |
| | | | AHSRALAGLAARGAAGCAADSVFFNVSVLDPTARLQLEARL |
| | | | QHLVAEILEREQSLALHALGYQLAFVLDSPSAYDAVAPSAAHL |
| | | | IDALYAEFLGGRVLTTPVVHRALFYASAVLRQPFLAGVPSAVQ |
| | | | RERARRSLLIASALCTSDVAAATNADLRTALARADHQKTLFW |
| | | | LPDHFSPCAASLRFDLDESVFILDALAQATRSETPVEVLAQQT |
| | | | HGLASTLTRWAHYNALIRAFVPEASHRCGGQSANVEPRILVP |
| | | | ITHNASYVVTHSPLPRGIGYKLTGVDVRRPLFLTYLTATCEGST |
| | | | RDIESKRLVRTQNQRDLGLVGAVFMRYTPAGEVMSVLLVDT |
| | | | DNTQQQIAAGPTEGAPSVFSSDVPSTALLLFPNGTVIHLLAFD |
| | | | TQPVAAIAPGFLAASALGVVMITAALAGILKVLRTSVPFFWRR |
| | | | E (SEQ ID NO: 17) |
| gH | tsQ26 | Q69075 | QLLYISKTYLLGRPPNASLPAPITVEPTAQPPPAVAPLKGLLHN |
| | | | PTASVLLRSRAWVTFSAVPDPEALTFPRGDNVATASHPSGPR |
| | | | DTPPPRPPVGARRHPTTELDITHLHNASTTWLATRGLLRSPG |
| | | | RYVYFSPSASTCPVGIWTTGELVLGCDSAGRARYGREF |
| | | | (SEQ ID NO: 20) |
| gH | Consensus | N/A | MGNGLWFVGVIILGXAWGQVHDWTEQTDPWFLDGLGMD |
| | Sequence | | RMYWRDTNTGRLWLPNTPDPQKPPRGFLAPPDELNLTTASL |
| | (see FIGS. | | PLLRWYEERFCFVLVTTAEFPRDPGQLLYIPKTYLLGRPPNASL |
| | 3A-3B) | | PAPTTVEPTAQPPPXVAPLKGLLHNPXASVLLRSRAWVTFSA |
| | | | VPDPEALTFPRGDNVATASHPSGPRDTPPPRPPVGARRHPT |
| | | | TELDITHLHNASTTWLATRGLLRSPGRYVYFSPSASTWPVGI |
| | | | VVTTGELVLGCDAALVRARYGREFMGLVISMHDSPPXEVMV |
| | | | VPAGQTLDRVGDPADENPPGALPGPPGGPRYRVFVLGSLTR |
| | | | ADNGSALDALRRVGGYPEEGTNYAQFLSRAYAEFFSGDAGA |
| | | | EQGPRPPLFWRLTGLLATSGFAFVNAAHANGAVCLSDLLGFL |
| | | | AHSRALAGLAARGAAGCAADSVFFNVSVLDPTARLQLEARL |
| | | | QHLVAEILEREQSLALHALGYQLAFVLDSPSAYDAVAPSAAHL |
| | | | IDALYAEFLGGRVXTTPVVHRALFYASAVLRQPFLAGVPSAV |
| | | | QRERARRSLLIASALCTSDVAAATNADLRTALARADHQKTLF |
| | | | WLPDHFSPCAASLRFDLDESVFILDALAQATRSETPVEVLAQ |
| | | | QTHGLASTLTRWAHYNALIRAFVPEASHRCGGQSANVEPRIL |
| | | | VPITHNASYVVTHSPLPRGIGYKLTGVDVRRPLFLTYLTATCEG |
| | | | STRDIESKRLVRTQNQRDLGLVGAVFMRYTPAGEVMSVLLV |
| | | | DTDNTQQQIAAGPTEGAPSVFSSDVPSTALLLFPNGTVIHLLA |
| | | | FDTQPVAAIAPGFLAASALGVVMITAALAGILKVLRTSVPFFW |
| | | | RRE (SEQ ID NO: 18) |
| | | | (X at residue 15 is A or V; X at residue 138 is A or 5; X at |
| | | | residue 150 is T or A; X at residue 284 is A or V; X at |
| | | | residue 510 is L or V) |
| gL | HG52 | P28278 | MGFVCLFGLVVMGAWGAWGGSQATEYVLRSVIAKEVGDIL |
| | | | RVPCMRTPADDVSWRYEAPSVIDYARIDGIFLRYHCPGLDTF |
| | | | LWDRHAQRAYLVNPFLFAAGFLEDLSHSVFPADTQETTTRR |
| | | | ALYKEIRDALGSRKQAVSHAPVRAGCVNFDYSRTRRCVGRR |
| | | | DLRPANTTSTWEPPVSSDDEASSQSKPLATQPPVLALSNAPP |
| | | | RRVSPTRGRRRHTRLRRN (SEQ ID NO: 21) |
| gL | 17 | P10185 | MGILGWVGLIAVGVLCVRGGLPSTEYVIRSRVAREVGDILKV |
| | | | PCVPLPSDDLDWRYETPSAINYALIDGIFLRYHCPGLDTVLWD |
| | | | RHAQKAYWVNPFLFVAGFLEDLSYPAFPANTQETETRLALYK |
| | | | EIRQALDSRKQAASHTPVKAGCVNFDYSRTRRCVGRQDLGP |
| | | | TNGTSGRTPVLPPDDEAGLQPKPLTTPPPIIATSDPTPRRDAA |
| | | | TKSRRRRPHSRRL (SEQ ID NO: 22) |

A “glycoprotein-derived peptide” as used herein refers to a substituted or unsubstituted fragment of an HSV-1 glycoprotein. In some embodiments, the glycoprotein-derived peptide is a substituted or unsubstituted fragment comprising at least 5, at least 6, at least 7, at least 8, at least 9, at least 10, at least 15, or at least 20 contiguous amino acids of a glycoprotein set forth in Table 1 above. In some embodiments, the glycoprotein-derived peptide is a substituted or unsubstituted fragment comprising up to about 40, up to 40, up to 39, up to 38, up to 37, up to 36, up to 35, up to 34, up to 33, up to 32, up to 31, up to 30, or up to about 30 contiguous amino acids of a glycoprotein set forth in Table 1 above. In some embodiments, the glycoprotein-derived peptide is a substituted or unsubstituted fragment comprising from 5, 6, 7, 8, 9, 10, 15, or 20, to about 30, 30, 31, 32, 33, 34, 35, 36, 37, 38, 39, 40, or about 40 contiguous amino acids of a glycoprotein set forth in Table 1 above. In some embodiments, the glycoprotein-derived peptide is a substituted or unsubstituted fragment comprising about 10%, about 20%, about 30%, about 40%, about 50%, about 60%, about 70%, about 80%, about 90%, or about 100% of the contiguous amino acids of a glycoprotein set forth in Table 1 above. Preferred fragments of the gB glycoprotein include, for example, residues 500-544 of P10211. Preferred fragments of the gH glycoprotein include, for example, residues 493-612 of P08356 and residues 493-612 of Q9DHD5.

As will be appreciated by those skilled in the art, the amino acids in the sequences described herein include, without limitation, both the D- and L-isomers of the naturally-occurring amino acids, as well as non-naturally occurring amino acids prepared by organic synthesis or other routes. Unless the context specifically indicates otherwise, the amino acid is intended to include amino acid analogs. The term “amino acid analog” or “non-natural amino acid” refers to a molecule which is structurally similar to an amino acid and which can be substituted for an amino acid in the formation of a crosslinked polypeptide and/or to allow attachment on the dendrimer surface. Amino acid analogs include, without limitation, compounds which are structurally identical to an amino acid except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine, or substitution or the carboxy group with an ester). Other suitable substitutions include, for example, replacing one or more alpha amino acids with a beta amino acid or gamma amino acid, substituting one or more charged residues with a residue of like charge, substituting one or more hydrophobic or hydrophilic residues with a residue of similar hydrophobicity/hydrophilicity, adding an organic moiety (e.g., a lipid), substituting the peptide bond with another covalent bond, etc.

The glycoprotein-derived peptide can be conjugated to the dendrimer at either the N-terminal or C-terminal end.

In at least one embodiment, the HSV-1 envelope glycoprotein-derived peptide binds to heparin sulfate.

Unless stated otherwise, suitable HSV-1 envelope glycoprotein-derived peptides include, for example, a substituted or unsubstituted glycoprotein-derived peptide shown in Table 2 below.

TABLE 2
HSV-1 Envelope Glycoprotein-Derived Peptides

| | Glycoprotein- | | |
|---|---|---|---|
| | Derived | | |
| | Peptide | | |
| Glycoprotein | Name | Description | Sequence |
| gB | gB8, gBhim, | Residues | FARLQFTYNHIQRHVRDMEGR (SEQ ID NO: 23) |
| | or | 503-523 of | |
| | gB503-523 | envelope | |
| | | glycoprotein | |
| | | B | |
| gB | HB168-186 | | VTVSQVWFGHRYSQFMGIF (SEQ ID NO: 24) |
| gB | HB287-305 | | FVLATGDFVYMSPFYGYRE (SEQ ID NO: 25) |
| gB | HB389-398 | | YGGSFRFSSDAISTTFTTN (SEQ ID NO: 26) |
| gB | HB441-459 | | YYLANGGFLIAYQPLLSNT (SEQ ID NO: 27) |
| gB | HB491-514 | | SVERIKTTSSIEFARLQFTYNHIQ (SEQ ID NO: 28) |
| gB | HB653-671 | | YAYSHQLSRADITTVSTFI (SEQ ID NO: 29) |
| gB | gBh or helix | | SIEFARLQFTYNHIQRHVNDMLGRVAIAWCELQNHELTLW |
| | 500-544 | | NEARK (SEQ ID NO: 30) |
| gB | gBhN or N- | | SIEFARLQFTYNHIQRHVNDMLGR (SEQ ID NO: 31) |
| | helix 500-523 | | |
| gB | gBhC or C- | | VAIAWCELQNHELTLWNEARK (SEQ ID NO: 32) |
| | helix 524-544 | | |
| gB | gBh1 | | FARLQFTYNHIQRHVNDMLGR (SEQ ID NO: 33) |
| gB | gBhs | | YNHIQRHVNDMLGR (SEQ ID NO: 34) |
| gB | gBh2 | | YNHIQRHVNDMLGRVAIAWCE (SEQ ID NO: 35) |
| gB | gBh2m | | YNHIQRHVNDMLGRVKKAWEE (SEQ ID NO: 36) |
| gB | gBh3 | | FARLQFTYNHIQRHVNDMLGRVAIAWCE |
| | | | (SEQ ID NO: 37) |
| gB | gBh3m | | FARLQFTYNHIQRHVNDMLGRVKKAWEE |
| | | | (SEQ ID NO: 38) |
| gB | gBh4 | | SIEFARLQFTYNHIQRHVNDMLGRVAIAWCELQNHE |
| | | | (SEQ ID NO: 39) |
| gB | BB181-198 | | IVTTTWAGSTYAAITNQY (SEQ ID NO: 40) |
| gB | BB525-548 | | AGGRVTTVSLAEFAALQFTHDHTR (SEQ ID NO: 41) |
| gB | BB664-678 | | ANHKRYFRFGADYVY (SEQ ID NO: 42) |
| gC | gC1 or | Residues | GSRVQIRCRFRNSTR (SEQ ID NO: 43) |
| | gC137-151 | 137-151 of | |
| | | envelope | |
| | | glycoprotein | |
| | | C | |
| gC | gC1ᵥ₁ | Variant of | GSRVQITCRFRNSTR (SEQ ID NO: 44) |
| gC1 | | | |
| gC | gC1ᵥ₂ | Variant of | GSRVQIRCTFRNSTR (SEQ ID NO: 45) |
| gC1 | | | |
| gC | gC1ᵥ₃ | Variant of | GSRVQIRCRFTNSTR (SEQ ID NO: 46) |
| gC1 | | | |
| gC | gC1ᵥ₄ | Variant of | GSRVQITCTFTNSTR (SEQ ID NO: 47) |
| gC1 | | | |
| gC | gC1ᵥ₅ | Variant of | GSTVQIRCRFRNSTT (SEQ ID NO: 48) |
| gC1 | | | |
| gC | gC1ᵥ₆ | Variant of | GSRTQIRCRFRNSTR (SEQ ID NO: 49) |
| gC1 | | | |
| gC | gC1ᵥ₇ | Variant of | GSRVQTRCRFRNSTR (SEQ ID NO: 50) |
| gC1 | | | |
| gC | gC1ᵥ₈ | Variant of | GSRVQIRCRTRNSTR (SEQ ID NO: 51) |
| gC1 | | | |
| gC | gC1ᵥ₉ | Variant of | GSRTQTRCRTRNSTR (SEQ ID NO: 52) |
| gC1 | | | |
| gH | PgH | Residues | AAHLIDALYAEFLGGRVLTT (SEQ ID NO: 53) |
| | | 493-512 of | |
| | | envelope | |
| | | glycoprotein | |
| | | H | |
| gH | gH625 | | NH₂-HGLASTLTRWAHYNALIRAF-PrA-CONH₂ |
| | | | (SEQ ID NO: 54) |
| gH | gH625-NBD | | NBD-HGLASTLTRWAHYNALIRAFX-CONH₂ |
| | | | (SEQ ID NO: 54) |
| gH | gH220-262 | | TWLATRGLLRSPGRYVYFSPSASTWPVGIWTTGELVLGCD |
| | | | AAL (SEQ ID NO: 55) |
| gH | gH381-420 | | RLTGLLATSGFAFVNAAHANGAVCLSDLLGFLAHSRALAG |
| | | | (SEQ ID NO: 56) |
| gH | gH493-537 | | AAHLIDALYAEFLGGRVLTTPVVHRALFYASAVLRQPFLAG |
| | | | VPSA (SEQ ID NO: 57) |
| gH | gH626-644 | | GLASTLTRWAHYNALIRAF (SEQ ID NO: 58) |
| gH | gH591-615 | | ASLRFDLDESVFILDALAQATRSET (SEQ ID NO: 59) |
| gH | gH601-625 | | VFILDALAQATRSETPVEVLAQQTH (SEQ ID NO: 60) |
| Unknown | g1 | Selected as | LRSRTKIIRIRH (SEQ ID NO: 61) |
| | | anti-heparin | |
| | | sulfate by | |
| | | peptide | |
| | | phage | |
| | | library | |
| Unknown | g1₁₀ | g1 10 Mer | RSRTKIIRIR (SEQ ID NO: 62) |
| | | seq | |
| Unknown | g2 | Selected as | MPRRRRIRRRQK (SEQ ID NO: 63) |
| | | anti-heparin | |
| | | sulfate by | |
| | | peptide | |
| | | phage | |
| | | library | |
| Unknown | g2₁₀ | g2 10 Mer | RRRRIRRRQK (SEQ ID NO: 64) |
| | | seq | |
| Unknown | g2ₘ₁ | g2 peptide | APRRRRIRRRQK (SEQ ID NO: 65) |
| | | mutant | |
| Unknown | g2ₘ₂ | g2 peptide | MARRRRIRRRQK (SEQ ID NO: 66) |
| | | mutant | |
| Unknown | g2ₘ₃ | g2 peptide | MPARRRIRRRQK (SEQ ID NO: 67) |
| | | mutant | |
| Unknown | g2ₘ₄ | g2 peptide | MPRARRIRRRQK (SEQ ID NO: 68) |
| | | mutant | |
| Unknown | g2ₘ₅ | g2 peptide | MPRRARIRRRQK (SEQ ID NO: 69) |
| | | mutant | |
| Unknown | g2ₘ₆ | g2 peptide | MPRRRAIRRRQK (SEQ ID NO: 70) |
| | | mutant | |
| Unknown | g2ₘ₇ | g2 peptide | MPRRRRARRRQK (SEQ ID NO: 71) |
| | | mutant | |
| Unknown | g2ₘ₈ | g2 peptide | MPRRRRIARRQK (SEQ ID NO: 72) |
| | | mutant | |
| Unknown | g2ₘ₃ | g2 peptide | MPRRRRIRARQK (SEQ ID NO: 73) |
| | | mutant | |
| Unknown | g2ₘ₁₀ | g2 peptide | MPRRRRIRRAQK (SEQ ID NO: 74) |
| | | mutant | |
| Unknown | g2ₘ₁₁ | g2 peptide | MPRRRRIRRRAK (SEQ ID NO: 75) |
| | | mutant | |
| Unknown | g2ₘ₁₂ | g2 peptide | MPRRRRIRRRQA (SEQ ID NO: 76) |
| | | mutant | |

In a preferred embodiment, the glycoprotein-derived peptide is selected from the group consisting of gB8, PgH, gC1, g1, and g2. gB8 corresponds to the long helical segment of glycoprotein gB and contains the heptad repeat sequence, which is typical of coiled-coil structures. This peptide presents a high antiviral activity. PgH is derived from the glycoprotein gH and exerts its antiviral activity by blocking viral rearrangements necessary for entry. Peptide gC1 is derived from glycoprotein gC, which mediates initial virus contact with cells by binding to heparin sulfate (HS) chains. gC1 overlaps a major part of the HS-binding site of gC and is able to inhibit HSV-1 infection. The two peptides g1 and g2 were selected as anti-heparin sulfate peptide by phage library.

In the case of the monofunctional peptidodendrimers, one type of HSV-1 envelope glycoprotein-derived peptide is conjugated to the dendrimer.

In the case of bifunctional peptidodendrimers, two types of HSV-1 envelope glycoprotein-derived peptides are conjugated to the dendrimer. In at least one embodiment, the two types of HSV-1 envelope glycoprotein-derived peptides are present at the same concentration.

In at least one embodiment, the monofunctional or bifunctional peptidodendrimer conjugate further comprises one or more therapeutic agents adsorbed to the peptidodendrimer conjugate. Suitable therapeutic agents include any known therapeutic agent useful against HSV-1, including, for example, anti-viral agents (e.g., acyclovir, valacyclovir, famciclovir, penciclovir).

Dendrimers have been extensively studied as vehicles for the delivery of therapeutics or as carriers for in vivo imaging (Lee et al., “Designing Dendrimers for Biological Applications,” Nat. Biotech. 23(12):1517-26 (2005); Esfand & Tomalia, “Poly(amidoamine) (PAMAM) Dendrimers: From Biomimicry to Drug Delivery and Biomedical Applications,” Drug Discov. Today 6(8):427-36 (2001); Sadler & Tam, “Peptide Dendrimers: Applications and Synthesis,” Rev. Mol. Biotechnol. 90:195-229 (2002); Cloninger, “Biological Applications of Dendrimers,” Curr. Opin. Chem. Biol. 6:742-48 (2002); Niederhafner et al., “Peptide Dendrimers,” J. Peptide Sci. 11:757-88 (2005); Tekade et al., “Dendrimers in Oncology: An Expanding Horizon,” Chem. Rev. 109(1):49-87 (2009), each of which is hereby incorporated by reference in its entirety). Dendrimers are highly branched macromolecules with well defined three-dimensional architectures (GEORGE R. NEWKOME ET AL., DENDRIMERS AND DENDRONS: CONCEPTS, SYNTHESIS, APPLICATIONS (2001), which is hereby incorporated by reference in its entirety). The appeal of dendrimers lies in their unique perfectly branched architectures, which affords them different properties than corresponding linear polymers of the same composition and molecular weights (Lee et al., “Designing Dendrimers for Biological Applications,” Nat. Biotech. 23(12):1517-26 (2005), which is hereby incorporated by reference in its entirety). As dendrimers increase in generation, they exponentially increase the number of termini, while only linearly increasing in radius; thus, the termini become more densely packed giving the entire structure a globular shape, where the termini radiate outwards from a central core.

The polyamide dendrimers according to this and all aspects of the present invention contain an amide dendrimer core and amide branches emanating from the core.

In at least one embodiment the peptidodendrimer conjugate has the formula:

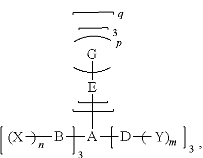

wherein:
A is an amide dendrimer core;
B, D, and E (if present) are each a moiety of formula

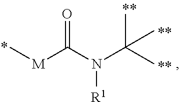

wherein:
*- is the point of attachment to A;
** is the point of attachment to X, Y, or G (if present), with the proviso that when m, n, and p are less than 3, ** can be a point of attachment to hydrogen;
M is an aromatic or aliphatic moiety;
each R¹ is selected from the group consisting of H and C₁₋₃ alkyl; and
each B, D, and E (if present) can be the same or different;
X, Y, and G (if present) are each independently a moiety of formula ***-Q-C(O)—NR²-L-Z—P, wherein:
   ***- is the point of attachment to B, D, or E (if present);
   Q is optionally present and, if present, is an aromatic or aliphatic moiety;
   each R² is selected from the group consisting of H and C₁₋₃ alkyl;
   each L is optionally present and, if present, is a linker;
   each Z is optionally present and, if present, is a spacer; and
   each P is an HSV-1 envelope glycoprotein-derived peptide (where, in the case of the monofunctional peptidodendrimer conjugates, each P is the same HSV-1 envelope glycoprotein-derived peptide; and, in the case of the bifunctional peptidodendrimer conjugates, each P is one of two different HSV-1 envelope glycoprotein-derived peptides);
      m, n, and p are the same and are each 1, 2, or 3; and
      q is 0 or 1.

Various types of amide dendrimer cores have been described in the art. Suitable cores include those described in Tarallo et al., Int'l J. Nanomed. 8:521-34 (2013); Carberry et al., Chem. Eur. J. 1813678-85 (2012); Jung et al., Macromolecules 44:9075-83 (2011); Ornelas et al., J. Am. Chem. Soc. 132:3923-31 (2010); Ornelas et al., Chem. Commun. 5710-12 (2009); Goyal et al., Adv. Synth. Catal. 350:1816-22 (2008); and Yoon et al., Org. Lett. 9:2051-54 (2007), each of which is hereby incorporated by reference in its entirety.

In at least one embodiment of the monofunctional or bifunctional peptidodendrimer conjugate, the amide dendrimer core A is a moiety of formula

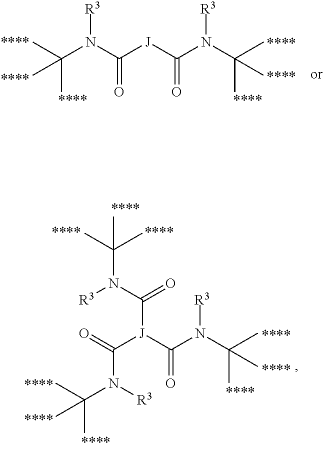

wherein
****- is the point of attachment to B, D, or E (if present); each R³ is selected from the group consisting of H and C₁₋₁₁ alkyl; and J is an aromatic or aliphatic moiety.

In at least one embodiment of the monofunctional or bifunctional peptidodendrimer conjugate, J is selected from the group consisting of C₁₋₂₀ alkyl, C₁₋₂₀ alkylene, trivalent C₁₋₂₀ alkane, C₂₋₂₀ alkenyl, C₂₋₂₀ alkenylene, trivalent C₂₋₂₀ alkene, C₂₋₂₀ alkynyl, C₂₋₂₀ alkynylene, trivalent C₂₋₂₀ alkyne, —C(O)—, —C(O)O—, —O—, —S—, —NH—, —N(R²⁰)—, —NHC(O)—, —N(R²⁰)C(O)—,

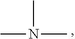

—Si(R²¹R²²)—, cycloalkyl, cycloalkylene, trivalent cycloalkane, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂. Suitable J moieties for use in the amide dendrimer core include, for example, those described in Tarallo et al., Int'l J. Nanomed. 8:521-34 (2013); Carberry et al., Chem. Eur. J. 1813678-85 (2012); Jung et al., Macromolecules 44:9075-83 (2011); Ornelas et al., J. Am. Chem. Soc. 132:3923-31 (2010); Ornelas et al., Chem. Commun. 5710-12 (2009); Goyal et al., Adv. Synth. Catal. 350:1816-22 (2008); and Yoon et al., Org. Lett. 9:2051-54 (2007), each of which is hereby incorporated by reference in its entirety.

In at least one embodiment of the monofunctional or bifunctional peptidodendrimer conjugate, J is a moiety of formula —(CR⁴R⁵)ₛ—, wherein s is 0 to 20 and each R⁴ and R⁵ are independently selected from the group consisting of H, alkyl, alkenyl, alkynyl, cycloalkyl, hydroxyalkyl, thiol, thioalkyl, alkylthioalkyl, alkoxy, aldehyde, ketone, acid, amine, amide, alcohol, heterocyclyl, aryl, heteroaryl, arylalkyl, and acyl. In at least one embodiment, at least one of R⁴ and R⁵ is a C₁₋₁₁ alkyl optionally substituted with from 1 to 3 substituents independently selected at each occurrence thereof from C₁₋₁₁ alkyl, halogen, —CN, —COOR⁶, —C(O)R⁷, —OR⁸, —NR⁹R¹⁰, —S(O)ₓR¹¹, —SR¹², and aryl; where R⁶, R⁷, R⁸, R⁹, R¹⁰, R¹¹, and R¹² are independently selected from the group consisting of H, C₁₋₁₁ alkyl, aryl, and heteroaryl; and x is 1 or 2.

In at least one embodiment of the monofunctional or bifunctional peptidodendrimer conjugate, A has the formula

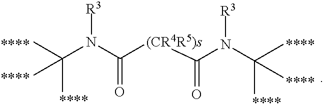

In at least one embodiment of the monofunctional or bifunctional peptidodendrimer conjugate, A is selected from the group consisting of

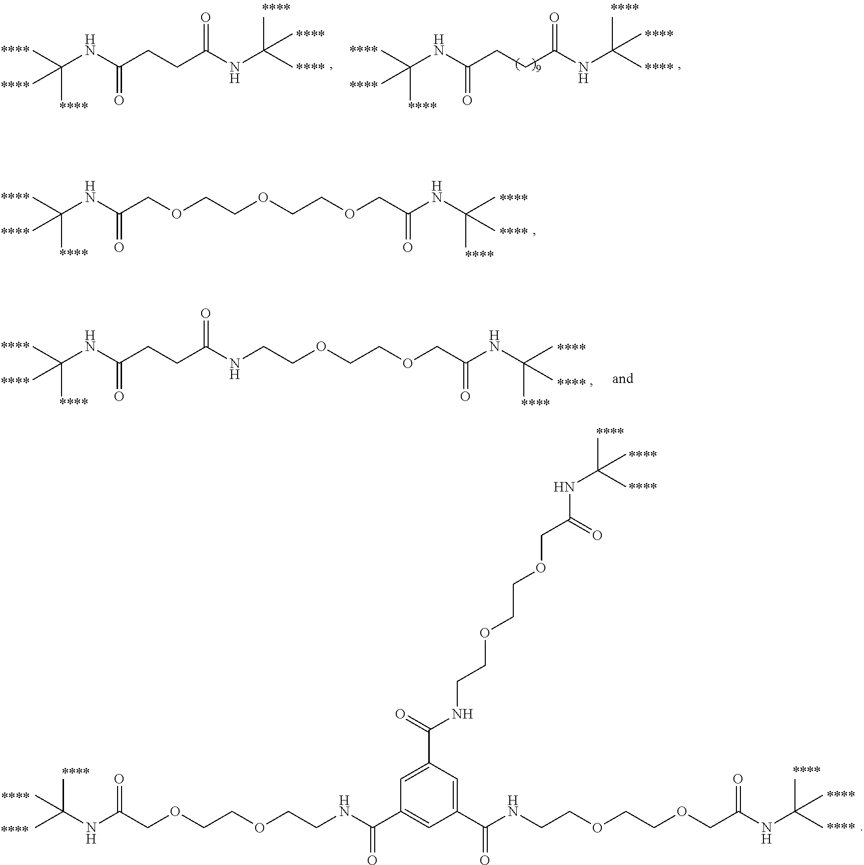

As will be understood by the skilled artisan, B, D, and E (if present) are dendrons that connect the core to outer branches.

In at least one embodiment of the monofunctional or bifunctional peptidodendrimer conjugate, M is selected from the group consisting of C₁₋₂₀ alkyl, C₁₋₂₀ alkylene, C₂₋₂₀ alkenyl, C₂₋₂₀ alkenylene, C₂₋₂₀ alkynyl, C₂₋₂₀ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂. Suitable M moieties for use in the dendron include, for example, those described in Tarallo et al., Int'l J. Nanomed. 8:521-34 (2013); Carberry et al., Chem. Eur. J. 1813678-85 (2012); Ornelas et al., Chem. Eur. J. 17:3619-29 (2011); Jung et al., Macromolecules 44:9075-83 (2011); Ornelas et al., J. Am. Chem. Soc. 132:3923-31 (2010); Ornelas et al., Chem. Commun. 5710-12 (2009); Goyal et al., Adv. Synth. Catal. 350:1816-22 (2008); and Yoon et al., Org. Lett. 9:2051-54 (2007), each of which is hereby incorporated by reference in its entirety.

In at least one embodiment of the monofunctional or bifunctional peptidodendrimer conjugate, M is a moiety of formula —(CR¹³R¹⁴)ₜ—, wherein t is 0 to 20 and each R¹³ and R¹⁴ are independently selected from the group consisting of H and C₁₋₃ alkyl.

In at least one embodiment of the monofunctional or bifunctional peptidodendrimer conjugate, at least one of B, D, and E (if present) is selected from the group consisting of

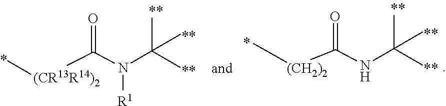

In at least one embodiment of the monofunctional or bifunctional peptidodendrimer conjugate, each B, D, and E (if present) are the same. In at least one embodiment of the monofunctional or bifunctional peptidodendrimer conjugate, each B, D, and E (if present) are different.

As will be understood by the skilled artisan, X, Y, and G (if present) are dendrons to which the peptide is conjugated.

In at least one embodiment of the monofunctional or bifunctional peptidodendrimer conjugate, Q is selected from the group consisting of C₁₋₂₀ alkyl, C₁₋₂₀ alkylene, C₂₋₂₀ alkenyl, C₂₋₂₀ alkenylene, C₂₋₂₀ alkynyl, C₂₋₂₀ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂. Suitable Q moieties for use in the dendron include, for example, those described in Tarallo et al., Int'l J. Nanomed. 8:521-34 (2013); Carberry et al., Chem. Eur. J. 1813678-85 (2012); Ornelas et al., Chem. Eur. J. 17:3619-29 (2011); Jung et al., Macromolecules 44:9075-83 (2011); Ornelas et al., J. Am. Chem. Soc. 132:3923-31 (2010); Ornelas et al., Chem. Commun. 5710-12 (2009); Goyal et al., Adv. Synth. Catal. 350:1816-22 (2008); and Yoon et al., Org. Lett. 9:2051-54 (2007), each of which is hereby incorporated by reference in its entirety.

In at least one embodiment of the monofunctional or bifunctional peptidodendrimer conjugate, Q is a moiety of formula —(CR¹⁵R¹⁶)ᵤ—, wherein u is 0 to 20 and each R¹⁵ and R¹⁶ are independently selected from the group consisting of H and C₁₋₃ alkyl.

In accordance with the present invention, X, Y, and G (if present) optionally include a linker L. The linker can include any suitable chemical moiety which can link N(R²) to Z. Typically, L is formed from a precursor that can be protected and deprotected in the presence of an amine and/or amide.

In at least one embodiment of the monofunctional or bifunctional peptidodendrimer conjugate, L is a saturated or unsaturated, branched or unbranched, carbon chain of from 1 to about 50 atoms in length, which can be optionally substituted throughout the chain and can include from 1 to 25 heteroatoms in the chain. Suitable optional substituents include, but are not limited to, —NO₂, —CN, halogen, oxo, C₁₋₆ alkyl, C₁₋₆ haloalkyl, C₁₋₆ hydroxyalkyl, C₁₋₆ alkoxy, C₁₋₆ alkoxyalkyl, C₃₋₆ cycloalkyl, C₄₋₇ cycloalkylalkyl, aryl, heteroaryl, —COOR⁹, —COR⁹, —C(O)NR⁹R¹⁰, —COONR⁹R¹⁰, —SO₂R⁹, —SO₂NR⁹R¹⁰, and —OR⁹. Suitable heteroatoms include, but are not limited to, O, S, N, and Si. A heteroatom, if present, may be directly bonded to Z or within the carbon chain.

In at least one embodiment of the monofunctional or bifunctional peptidodendrimer conjugate, L has the formula —R¹⁷R¹⁸R¹⁹—, wherein each R¹⁷, R¹⁸, and R¹⁹ is optionally present and, if present, is independently selected from the group consisting of C₁₋₆ alkyl, C₁₋₆ alkylene, C₂₋₆ alkenyl, C₂₋₆ alkenylene, C₂₋₆ alkynyl, C₂₋₆ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein the C₁₋₆ alkyl, C₁₋₆ alkylene, C₂₋₆ alkenyl, C₂₋₆ alkenylene, C₂₋₆ alkynyl, C₂₋₆ alkynylene, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl can be optionally substituted with from 1 to 3 substituents independently selected at each occurrence thereof from C₁₋₁₁ alkyl, halogen, —CN, —COOR⁶, —C(O)R⁷, —OR⁸, —NR⁹R¹⁰, —S(O)ₓR¹¹, —SR¹² and aryl, wherein R⁶, R⁷, R⁸, R⁹, R¹⁰, R¹¹, and R¹² are independently selected from the group consisting of H, C₁₋₁₁ alkyl, aryl, and heteroaryl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂; and x is 1 or 2.

In accordance with the present invention, the X, Y, and G (if present) optionally include a spacer Z. The term “spacer” refers to a connecting group of a predetermined length being at least divalent.

In at least one embodiment of the monofunctional or bifunctional peptidodendrimer conjugate, Z is formed from a bioconjugation reaction. A variety of bioconjugation reactions can be used for the preparation of the monofunctional or bifunctional peptidodendrimer conjugates according to the present invention. These reactions can produce a wide variety of spacers that can be used in accordance with the present invention. Suitable bioconjugation reactions include, for example, click reactions, Staudinger ligation (e.g., Saxon & Bertozzi, Science 287(5460):2007 (2000), which is hereby incorporated by reference in its entirety), Schiff base chemistry (e.g., Yamgar et al., J. Chem. Pharm. Res. 2(5):216-24 (2010), which is hereby incorporated by reference in its entirety), reactions involving the thiol group of a cytosine residue, reactions involving lysine residues, Diels-Alder reactions (e.g., Corey et al., Angew. Chem. Int'l Ed. 41:1650-67(2002), which is hereby incorporated by reference in its entirety), and various other bioconjugation reactions (e.g., as described in GREG T. HERMANSON, BIOCONJUGATE TECHNIQUES (3d ed. 2013), which is hereby incorporated by reference in its entirety).

In some embodiments, Z is formed by a click reaction. A suitable click reaction is a 1,3-dipolar cycloaddition reaction. Click reactions of this type involve, for example, the coupling of two different moieties (e.g., a peptide and a functional group, a first functional group and a second functional group) via a 1,3-dipolar cycloaddition reaction between an alkyne moiety (or equivalent thereof) on the surface of the first moeity and an azide moiety (or equivalent thereof) or any active end group (such as, for example, a primary amine end group, a hydroxyl end group, a carboxylic acid end group, a thiol end group, etc.) on the second moiety. “Click chemistry” is an attractive coupling method because, for example, it can be performed with a wide variety of solvent conditions including aqueous environments. For example, the stable triazole ring that results from coupling the alkyne with the azide in the 1,3-dipolar cycloaddition reaction is frequently achieved at quantitative yields and is considered to be biologically inert (see, e.g., Rostovtsev et al., Angewandte Chem. Int'l Ed. 41(14):2596 (2002); Wu et al., Angewandte Chem. Int'l Ed. 43(30):3928-32 (2004), each of which is hereby incorporated by reference in its entirety). As will be apparent to the skilled artisan, other click reactions may also be used to form spacer Z.

In at least one embodiment of the monofunctional or bifunctional peptidodendrimer conjugate, spacer Z is propargylglycine.

In at least one embodiment of the monofunctional or bifunctional peptidodendrimer conjugate, at least one of X, Y, and G (if present) is selected from the group consisting of ***—(CR¹⁵R¹⁶)₂—CO—NR²-L-Z—P, ***—(CH₂)₂—CO—NH—Z—P, ***—(CH₂)₂—CO—NH—C—P,

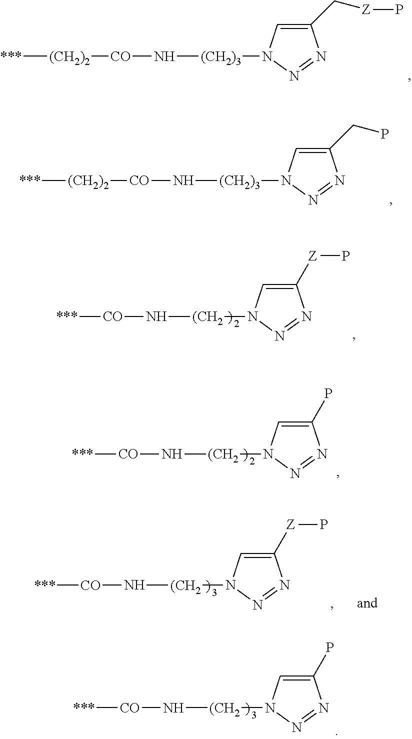

In at least one embodiment of the bifunctional peptidodendrimer conjugate, E and G are absent (i.e., q is 0); each X is ***—(CR¹⁵R¹⁶)₂—CO—NR²-L-Z—P¹, where P¹ is one of the HSV-1 envelope glycoprotein-derived peptides; and each Y is ***—(CR¹⁵R¹⁶)₂—CO—NR²-L-Z—P², where P² is the other of the HSV-1 envelope glycoprotein-derived peptides. In at least one embodiment of the bifunctional peptidodendrimer conjugate, X is ***—(CH₂)₂—CO—NH—Z—P¹ and Y is

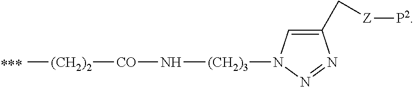

In at least one embodiment of the monofunctional or bifunctional peptidodendrimer conjugate, each X, Y, and G (if present) are the same. In at least one embodiment of the monofunctional or bifunctional peptidodendrimer conjugate, each X, Y, and G (if present) are different.

As used herein, the following terms, unless otherwise indicated, shall be understood to have the following meanings. If not defined otherwise herein, all technical and scientific terms used herein have the same meaning as is commonly understood by one of ordinary skill in the art to which this invention belongs. In the event that there is a plurality of definitions for a term herein, those in this section prevail unless stated otherwise.

The term “alkyl” means an aliphatic hydrocarbon group which may be straight or branched having about 1 to about 20 (e.g., 1, 2, 3, 4, 5, 6, 7, 8, 9, 10, 11, 12, 13, 14, 15, 16, 17, 18, 19, 20) carbon atoms in the chain, unless otherwise specified. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl. An alkylene is a divalent, straight or branched chain alkane group.

The term “alkenyl” means an aliphatic hydrocarbon group containing a carbon-carbon double bond and which may be straight or branched having about 2 to about 20 (e.g., 2, 3, 4, 5, 6, 7, 8, 9, 10, 11, 12, 13, 14, 15, 16, 17, 18, 19, 20) carbon atoms in the chain. Preferred alkenyl groups have 2 to about 6 (e.g., 2, 3, 4, 5, 6) carbon atoms in the chain. Exemplary alkenyl groups include ethenyl, propenyl, n-butenyl, and i-butenyl. An alkenylene is a divalent, straight or branched chain alkene group.

The term “alkynyl” means an aliphatic hydrocarbon group containing a carbon-carbon triple bond and which may be straight or branched having about 2 to about 20 (e.g., 2, 3, 4, 5, 6, 7, 8, 9, 10, 11, 12, 13, 14, 15, 16, 17, 18, 19, 20) carbon atoms in the chain. Preferred alkynyl groups have 2 to about 6 (e.g., 2, 3, 4, 5, 6) carbon atoms in the chain. Exemplary alkynyl groups include ethynyl, propynyl, n-butynyl, 2-butynyl, 3-methylbutynyl, and n-pentynyl. An alkynylene is a divalent, straight or branched chain alkyne.

The term “cycloalkyl” refers to a non-aromatic saturated or unsaturated mono- or polycyclic ring system which may contain 3 to 6 (e.g., 3, 4, 5, or 6) carbon atoms, and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane. A cycloalkylene is a divalent, straight or branched chain cycloalkane group.

The term “hydroxyalkyl” means an alkyl group is substituted with one or more hydroxy substituents, wherein the alkyl group is as herein described. A hydroxyalkylene is a divalent, straight or branched chain hydroxyalkane group.

The term “thioalkyl” means an alkyl group is substituted with one or more mecaptan (thiol) substituents, wherein the alkyl group is as herein described.

The term “alkylthioalkyl” means a thioalkyl group is substituted with one or more alkyl substituents, wherein the alkyl group is as herein described. Particularly, the thiol group of the thioalkyl can be substituted with one or more alkyl substituents.

As used herein, the term “heterocyclyl” refers to a stable 3- to 18-membered (e.g., 3-, 4-, 5-, 6-, 7-, 8-, 9-, 10-, 11-, 12-, 13-, 14-, 15-, 16-, 17-, or 18-membered) ring system that consists of carbon atoms and from one to five (e.g., 1, 2, 3, 4, or 5) heteroatoms selected from the group consisting of nitrogen, oxygen, sulfur, and silicon. The heterocyclyl may be a monocyclic or a polycyclic ring system, which may include fused, bridged, or spiro ring systems; and the nitrogen, carbon, sulfur, or silicon atoms in the heterocyclyl may be optionally oxidized; the nitrogen atom may be optionally quaternized; and the ring may be partially or fully saturated. Representative monocyclic heterocyclyls include piperidine, piperazine, pyrimidine, morpholine, thiomorpholine, pyrrolidine, tetrahydrofuran, pyran, tetrahydropyran, oxetane, and the like. Representative polycyclic heterocyclyls include indole, isoindole, indolizine, quinoline, isoquinoline, purine, carbazole, dibenzofuran, chromene, xanthene, and the like.

As used herein, the term “aryl” refers to an aromatic monocyclic or polycyclic ring system containing from 6 to 19 (e.g., 6, 7, 8, 9, 10, 11, 12, 13, 14, 15, 16, 17, 18, or 19) carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

As used herein, “heteroaryl” refers to an aromatic ring radical which consists of carbon atoms and from one to five (e.g., 1, 2, 3, 4, or 5) heteroatoms selected from the group consisting of nitrogen, oxygen, sulfur, and silicon. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. Additional heteroaryls are described in COMPREHENSIVE HETEROCYCLIC CHEMISTRY: THE STRUCTURE, REACTIONS, SYNTHESIS AND USE OF HETEROCYCLIC COMPOUNDS (Katritzky et al. eds., 1984), which is hereby incorporated by reference in its entirety.

The term “arylalkyl” refers to a moiety of the formula —R^{a}R^{b} where R^{a} is an alkyl or cycloalkyl as defined above and R^{b} is an aryl or heteroaryl as defined above.

As used herein, the term “acyl” means a moiety of formula R-carbonyl, where R is an alkyl, cycloalkyl, aryl, or heteroaryl as defined above. Exemplary acyl groups include formyl, acetyl, propanoyl, benzoyl, and propenoyl.

The term “halogen” means fluorine, chlorine, bromine, or iodine.

The term “alkoxy” means groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present patent application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

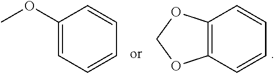

One aspect of the present invention relates to a monofunctional peptidodendrimer conjugate comprising: a polyamide dendrimer conjugated with an HSV-1 envelope glycoprotein-derived peptide, wherein the peptide is a substituted or unsubstituted peptide selected from the group consisting of gB8, PgH, gC1, g1, and g2.

In at least one embodiment of this aspect of the present invention, the peptide is gB8. In at least one embodiment of this aspect of the present invention, the peptide is unsubstituted.

In at least one embodiment of this aspect of the present invention, the monofunctional peptidodendrimer conjugate is:

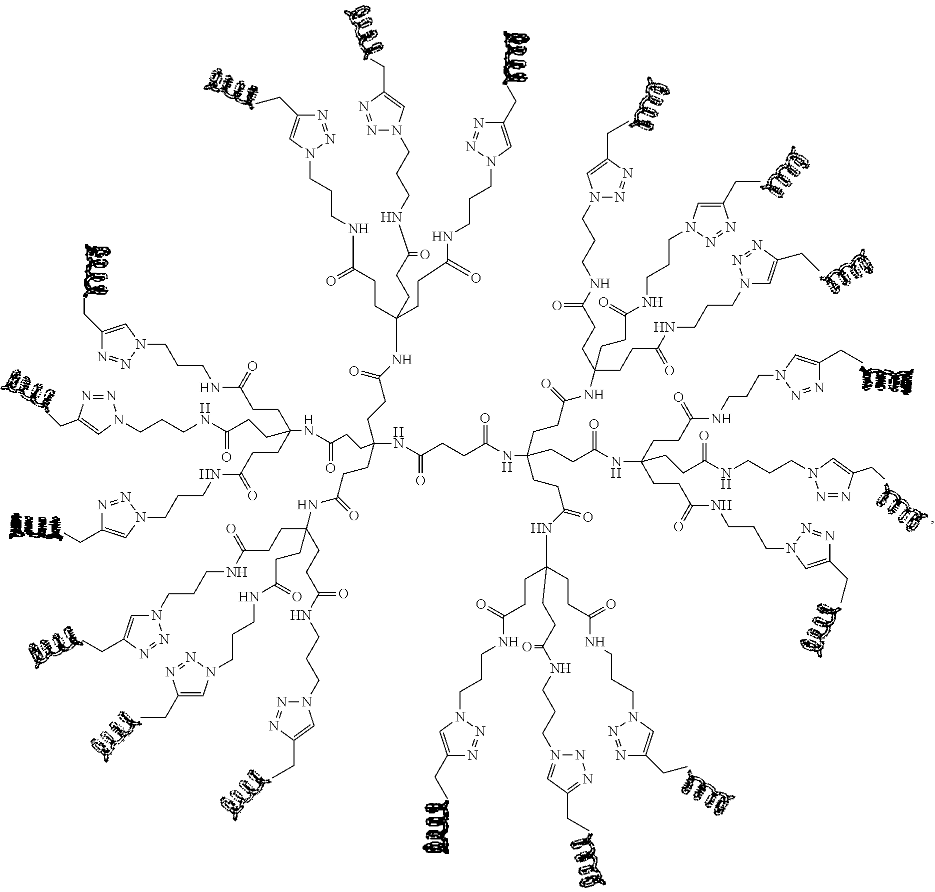

where each

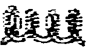

is the substituted or unsubstituted peptide. In a preferred embodiment, each

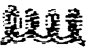

is a substituted or unsubstituted gB8. In a preferred embodiment, gB8 is unsubstituted.

Another aspect of the present invention relates to a pharmaceutical composition comprising, in a pharmaceutically acceptable vehicle, (i) a first monofunctional peptidodendrimer conjugate comprising: a polyamide dendrimer conjugated with a first HSV-1 envelope glycoprotein-derived peptide; and (ii) a second monofunctional peptidodendrimer conjugate comprising: a polyamide dendrimer conjugated with a second HSV-1 envelope glycoprotein-derived peptide; wherein the first and second HSV-1 envelope glycoprotein-derived peptides are different.

In at least one embodiment of this aspect of the present invention, the first HSV-1 envelope glycoprotein-derived peptide and the second HSV-1 envelope glycoprotein-derived peptide are each a substituted or unsubstituted peptide selected from the group consisting of the peptides set forth in Table 2 above.

In at least one embodiment of this aspect of the present invention, the first HSV-1 envelope glycoprotein-derived peptide is a substituted or unsubstituted peptide selected from the group consisting of gB8, PgH, gC1, g1, and g2. In at least one embodiment, the first peptide is a substituted or unsubstituted gB8. In at least one embodiment, the first peptide is a substituted or unsubstituted gB8 and the second peptide is a substituted or unsubstituted peptide selected from the group consisting of PgH, gC1, g1, and g2.

In at least one embodiment of this aspect of the present invention, the first HSV-1 envelope glycoprotein-derived peptide is a substituted or unsubstituted peptide selected from the group consisting of gB8, PgH, gC1, g1, and g2 and the second HSV-1 envelope glycoprotein-derived peptide is a substituted or unsubstituted peptide selected from the group consisting of the peptides set forth in Table 2 above. In at least one embodiment, one of the peptides is a substituted or unsubstituted gB8.

In at least one embodiment of this aspect of the present invention, the first and second peptides are derived from the same HSV-1 envelope glycoprotein.

In at least one embodiment of this aspect of the present invention, the first and second peptides are derived from different HSV-1 envelope glycoproteins.

Another aspect of the present invention relates to a bifunctional peptidodendrimer conjugate comprising: a polyamide dendrimer conjugated with two different HSV-1 envelope glycoprotein-derived peptides.

In at least one embodiment of this aspect of the present invention, both HSV-1 envelope glycoprotein-derived peptides are a substituted or unsubstituted peptide selected from the group consisting of the peptides set forth in Table 2 above.

In at least one embodiment of this aspect of the present invention, both HSV-1 envelope glycoprotein-derived peptides are a substituted or unsubstituted peptide selected from the group consisting of gB8, PgH, gC1, g1, and g2. In at least one embodiment, one of the peptides is a substituted or unsubstituted gB8. In at least one embodiment, one of peptides is a substituted or unsubstituted gB8 and the other peptide is a substituted or unsubstituted peptide selected from the group consisting of PgH, gC1, g1, and g2.

In at least one embodiment of this aspect of the present invention, the one of the HSV-1 envelope glycoprotein-derived peptides is a substituted or unsubstituted peptide selected from the group consisting of gB8, PgH, gC1, g1, and g2 and the other HSV-1 envelope glycoprotein-derived peptide is a substituted or unsubstituted peptide selected from the group consisting of the peptides set forth in Table 2 above. In at least one embodiment, one of the peptides is a substituted or unsubstituted gB8.

In at least one embodiment of this aspect of the present invention, the two different peptides are derived from the same HSV-1 envelope glycoprotein.

In at least one embodiment of this aspect of the present invention, the two different peptides are derived from different HSV-1 envelope glycoproteins.

In at least one embodiment of this aspect of the present invention, the bifunctional peptidodendrimer conjugate is:

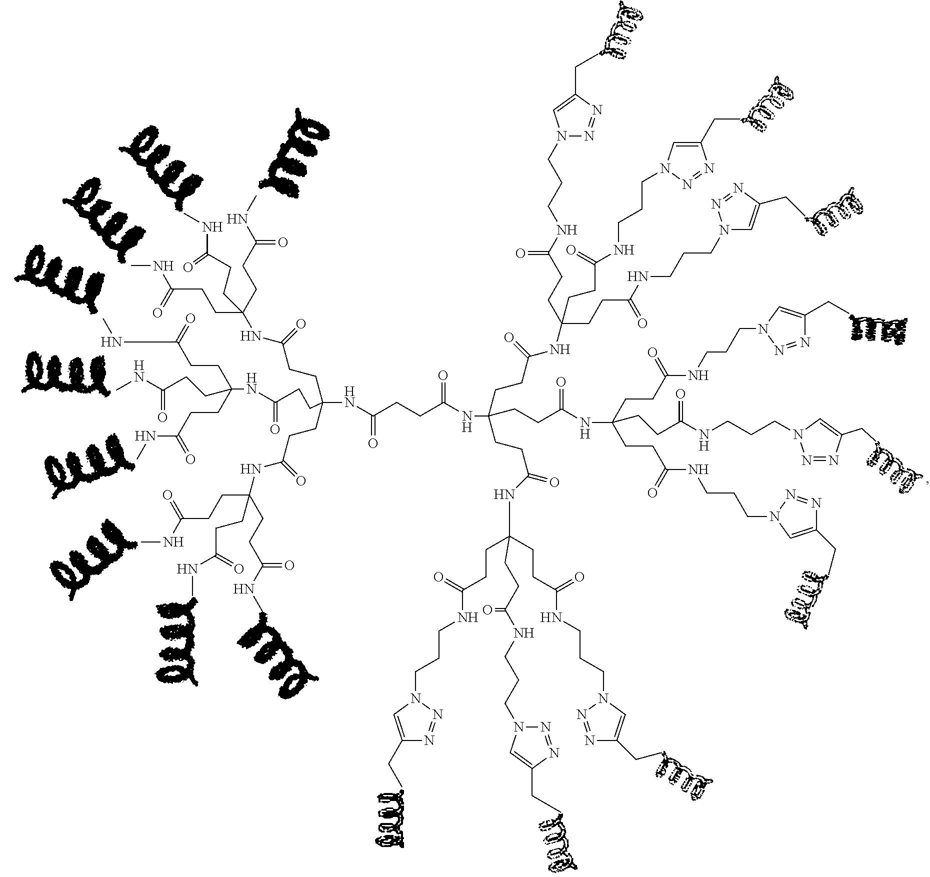

where each

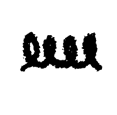

is one of the two HSV-1 glycoprotein-derived peptides and each

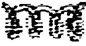

and is the other of the two HSV-1 glycoprotein-derived peptides. In at least one embodiment,

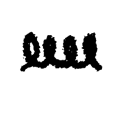

is gB8 and

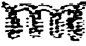

### is PgH.

Peptidodendrimer conjugates of the present invention may be made using methods in the art. Suitable methods include those described in Example 2 (monofunctional peptidodendrimer conjugates) and Example 10 (bifunctional peptidodendrimer conjugates) below.

Also encompassed by the present invention is a pharmaceutical formulation that includes a peptidodendrimer conjugate of the present invention and a pharmaceutically acceptable vehicle.

Suitable pharmaceutical formulations include the peptidodendrimer conjugate(s) and any pharmaceutically acceptable adjuvants, carriers, solutions, suspensions, emulsions, excipients, powders, and/or stabilizers, and can be in solid or liquid form, such as tablets, capsules, powders, solutions, suspensions, or emulsions. The compositions preferably contain from about 0.01 to about 99 weight percent, more preferably from about 2 to about 60 weight percent, of the peptidodendrimer conjugate(s) together with the adjuvants, carriers and/or excipients. The amount of active compound in such therapeutically useful compositions is such that a suitable dosage unit will be obtained.

In addition, the pharmaceutical formulations of the present invention may further comprise one or more pharmaceutically acceptable diluents or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agaragar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulfate, talc, as well as high molecular weight polyethylene glycols.

For oral therapeutic administration, the peptidodendrimer conjugate(s) may be incorporated with excipients and used in the form of tablets, capsules, elixirs, suspensions, syrups, and the like. Such compositions and preparations should contain at least 0.1% of the peptidodendrimer conjugate(s). The percentage of the peptidodendrimer conjugate(s) in these compositions may, of course, be varied and may conveniently be between about 2% to about 60% of the weight of the unit. The amount of the peptidodendrimer conjugate(s) in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient(s), sucrose as a sweetening agent, methyl and propylparabens as preservatives, a dye, and flavoring such as cherry or orange flavor.

Solutions or suspensions of the peptidodendrimer conjugate(s) (for example, for parenteral administration) can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycol or polyethylene glycol, are preferred liquid carriers, particularly for injectable solutions. Under ordinary conditions of storage and use, these preparations contain a preservative to prevent the growth of microorganisms.

Pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In at least one embodiment, the pharmaceutical formulation comprises a monofunctional peptidodendrimer conjugate as described above.

In at least one embodiment, the pharmaceutical formulation comprises a bifunctional peptidodendrimer conjugate as described above.

In at least one embodiment, the pharmaceutical formulation comprises a monofunctional peptidodendrimer conjugate and a bifunctional peptidodendrimer conjugate.

Pharmaceutical formulations include (i) those that contain monofunctional peptidodendrimer conjugates that are all the same, (i) those that contain different monofunctional peptidodendrimer conjugates, (iii) those that contain bifunctional peptidodendrimer conjugates that are all the same, (iv) those that contain different bifunctional peptidodendrimer conjugates, and (v) combinations of (i)(iv). In this context “the same” and “different” can refer to the architecture of the dendrimer in the peptidodendrimer conjugates, the HSV-1 glycoprotein-derived peptide(s) present in the peptidodendrimer conjugates, or both.

Another aspect of the present invention relates to methods of using the peptidodendrimer complexes described herein.

One embodiment of this aspect of the present invention relates to a method of treating or preventing HSV-1 infection in a subject. This method involves administering to the subject, under conditions effective to treat or prevent HSV-1 infection:
(i) a monofunctional peptidodendrimer conjugate comprising: a polyamide dendrimer conjugated with an HSV-1 envelope glycoprotein-derived peptide, wherein the peptide is a substituted or unsubstituted peptide selected from the group consisting of gB8, PgH, gC1, g1, and g2;
(ii) (a) a first monofunctional peptidodendrimer conjugate comprising: a polyamide dendrimer conjugated with a first HSV-1 envelope glycoprotein-derived peptide and (b) a second monofunctional peptidodendrimer conjugate comprising: a polyamide dendrimer conjugated with a second HSV-1 envelope glycoprotein-derived peptide, wherein the first and second HSV-1 envelope glycoprotein-derived peptides are different;
(iii) a bifunctional peptidodendrimer conjugate as described herein; or
(iv) a combination thereof.

The present invention provides for both prophylactic and therapeutic methods of treating a subject infected with HSV-1 or at risk of (or susceptible to) a HSV-1 infection. As used herein, the term “treatment” is defined as the application or administration of a therapeutic agent to a patient, or application or administration of a therapeutic agent to an isolated tissue or cell line from a patient, who has a disease, a symptom of disease, or a predisposition toward a disease, with the purpose to cure, heal, alleviate, relieve, alter, remedy, ameliorate, improve, or affect the disease, the symptoms of disease, or the predisposition toward disease. Infections that can be treated using the present method include, for example, oro-facial herpes, herpes labialis, herpetic esophagitis, herpes gingivostomatitis, HSV-1-mediated genital lesions, herpetic whitlow, herpes gladiatorum, keratitis and keratoconjuntivitis of the eye, eczema herpeticum, and HSV-1-mediated diseases (e.g., meningitis, encephalitis, myelitis, vasculopathy, ganglioneuritis, retinal necrosis, and optic neuritis).

As will be apparent to the skilled artisan, the present method can further involve selecting a subject infected with HSV-1 or at risk of (or susceptible to) a HSV-1 infection.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline (e.g., cats) or canine (e.g., dogs) subjects, farm animals, such as but not limited to bovine (e.g., cows), equine (e.g., horses), caprine (e.g., goats), ovine (e.g., sheep), and porcine (e.g., pigs) subjects, wild animals (whether in the wild or in a zoological garden), research animals, such as mice, rats, rabbits, guinea pigs, goats, sheep, pigs, dogs, cats, horses, cows, camels, llamas, monkeys, zebrafish etc., avian species, such as chickens, turkeys, songbirds, etc., i.e., for veterinary medical use.

In at least one embodiment, the subject is a mammal, fish, or bird. In at least one embodiment, the subject is selected from the group consisting of felines, canines, bovines, equines, camelids, caprines, ovines, porcines, rodents, leporids, primates, zebrafish, poultry, and songbirds. In at least one embodiment, the subject is selected from the group consisting of cats, dogs, cows, horses, camels, llamas, goats, sheep, pigs, mice, rats, guinea pigs, rabbits, monkeys, zebrafish, chickens, turkeys, and songbirds. In at least one embodiment, the subject is a human subject, a mouse, a rabbit, a guinea pig, or a zebrafish. Preferably, the subject is human.

Another embodiment according to this aspect of the present invention relates to a method of inhibiting entry of HSV-1 into a host cell. This method involves contacting the host cell, under conditions effective to inhibit entry of HSV-1 into the host cell, with:
(i) a monofunctional peptidodendrimer conjugate comprising: a polyamide dendrimer conjugated with an HSV-1 envelope glycoprotein-derived peptide, wherein the peptide is a substituted or unsubstituted peptide selected from the group consisting of gB8, PgH, gC1, g1, and g2;
(ii) (a) a first monofunctional peptidodendrimer conjugate comprising: a polyamide dendrimer conjugated with a first HSV-1 envelope glycoprotein-derived peptide and (b) a second monofunctional peptidodendrimer conjugate comprising: a polyamide dendrimer conjugated with a second HSV-1 envelope glycoprotein-derived peptide, wherein the first and second HSV-1 envelope glycoprotein-derived peptides are different;
(iii) a bifunctional peptidodendrimer conjugate as described herein; or
(iv) a combination thereof.

Suitable cells according to the methods of the present invention include, without limitation, mammalian cells, fish cells, or avian cells. In at least one embodiment, the cell is a cell of an animal selected from the group consisting of felines, canines, bovines, equines, camelids, caprines, ovines, porcines, rodents, leporids, primates, zebrafish, poultry, and songbirds. In at least one embodiment, the cell is a cell of an animal selected from the group consisting of cats, dogs, cows, horses, camels, llamas, goats, sheep, pigs, mice, rats, guinea pigs, rabbits, monkeys, zebrafish, chickens, turkeys, and songbirds. In at least one embodiment, the cell is a human cell, a mouse cell, a rabbit cell, a guinea pig cell, or a zebrafish cell. Preferably, the cell is a human cell.

Suitable host cells include, for example, immune system cells, neuronal cells, epithelial cells, mucosal cells, oral cells, ocular cells, and fibroblasts. Suitable immune system cells include, without limitation, monocytes, macrophages, dendritic cells, and T lymphocytes. Suitable epithelial cells include, without limitation, those of the mouth, genitals, anus, eyes, esophagus, trachea, arms, and legs. Suitable ocular cells include, without limitation, human conjunctival epithelial cells, corneal fibroblasts, and trabecular meshwork cells.

The host cell of the present method has on its surface at least one HSV-1 receptor (e.g., heparin sulfate, herpes virus entry mediator, nectin-1, nectin-2, 3-0 sulfated heparin sulfate, a gD-receptive glycosaminoglycan, paired immunoglobulin-like type 2 receptor-α (“PILR-α”), B5, αvβ3 integrin, myelin associated glycoprotein (“MAG”), non-muscle myosin heavy chain IIA (NMHC-IIA)).

Contacting (including administering) according to the methods of the present invention can be carried out using methods that will be apparent to the skilled artisan, and can be done in vitro or in vivo.

One approach for delivering agents to cells involves the use of liposomes. Basically, this involves providing a liposome which includes agent(s) to be delivered, and then contacting the target cell, tissue, or organ with the liposomes under conditions effective for delivery of the agent to the cell, tissue, or organ. This liposome delivery system can also be made to accumulate at a target organ, tissue, or cell via active targeting (e.g., by incorporating an antibody or hormone on the surface of the liposomal vehicle). This can be achieved according to known methods.

Another approach for delivery of peptide-containing agents (e.g., peptidodendrimer conjugates of the present invention) involves the conjugation of the desired agent to a polymer that is stabilized to avoid enzymatic degradation of the conjugated peptide. Conjugated proteins or polypeptides of this type are described in U.S. Pat. No. 5,681,811 to Ekwuribe, which is hereby incorporated by reference in its entirety.

Yet another approach for delivery of agents involves preparation of chimeric agents according to U.S. Pat. No. 5,817,789 to Heartlein et al., which is hereby incorporated by reference in its entirety. The chimeric agent can include a ligand domain and the agent (e.g., a peptidodendrimer conjugate of the present invention). The ligand domain is specific for receptors located on a target cell. Thus, when the chimeric agent is delivered intravenously or otherwise introduced into blood or lymph, the chimeric agent will adsorb to the targeted cell.

Peptidodendrimer conjugates of the present invention may be delivered directly to the targeted cell/tissue/organ.

Additionally and/or alternatively, the peptidodendrimer conjugate(s) may be administered to a non-targeted area along with one or more agents that facilitate migration of the peptidodendrimer conjugate(s) to a targeted tissue, organ, or cell. As will be apparent to one of ordinary skill in the art, the peptidodendrimer conjugate(s) itself can be modified to facilitate its transport to a target tissue, organ, or cell, including its transport across the blood-brain barrier. Some example target cells include the host cells described above. Some example tissues and/or organs include, for example, mouth, genitals, anus, skin, eyes, brain, arms, legs, and mucous membranes.

In vivo administration can be accomplished either via systemic administration to the subject or via targeted administration to affected tissues, organs, and/or cells, as described above. Typically, the therapeutic agent (i.e., peptidodendrimer conjugate of the present invention) will be administered to a patient in a vehicle that delivers the therapeutic agent(s) to the target cell, tissue, or organ. Typically, the therapeutic agent will be administered as a pharmaceutical formulation, such as those described above.

Exemplary routes of administration include, without limitation, orally, topically, transdermally, parenterally, subcutaneously, intravenously, intramuscularly, intraperitoneally, intraventricularly, and intralesionally; by intratracheal inoculation, aspiration, airway instillation, aerosolization, nebulization, intranasal instillation, oral or nasogastric instillation, intraperitoneal injection, intravascular injection, intravenous injection, intra-arterial injection (such as via the pulmonary artery), intramuscular injection, and intrapleural instillation; by application to mucous membranes (such as that of the nose, throat, bronchial tubes, genitals, and/or anus); and by implantation of a sustained release vehicle.

For use as aerosols, peptidodendrimer conjugate(s) of the present invention in solution or suspension may be packaged in a pressurized aerosol container together with suitable propellants, for example, hydrocarbon propellants like propane, butane, or isobutane with conventional adjuvants. The peptidodendrimer conjugate(s) of the present invention also may be administered in a non-pressurized form.

Exemplary delivery devices include, without limitation, nebulizers, atomizers, liposomes (including both active and passive drug delivery techniques) (Wang & Huang, Proc. Nat'l Acad. Sci. USA 84:7851-5 (1987); Bangham et al., J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al.; Wolff et al., Biochim. Biophys. Acta 802:259-73 (1984), each of which is hereby incorporated by reference in its entirety), transdermal patches, implants, implantable or injectable protein depot compositions, and syringes. Other delivery systems which are known to those of skill in the art can also be employed to achieve the desired delivery of the peptidodendrimer conjugate(s) to the desired organ, tissue, or cells in vivo to effect this aspect of the present invention.

Contacting (including in vivo administration) can be carried out as frequently as required and for a duration that is suitable to provide the desired effect. For example, contacting can be carried out once or multiple times, and in vivo administration can be carried out with a single sustained-release dosage formulation or with multiple (e.g., daily) doses.

The amount to be administered will, of course, vary depending upon the particular conditions and treatment regimen. The amount/dose required to obtain the desired effect may vary depending on the agent, formulation, cell type, culture conditions (for ex vivo embodiments), the duration for which treatment is desired, and, for in vivo embodiments, the individual to whom the agent is administered.

Effective amounts can be determined empirically by those of skill in the art. For example, this may involve assays in which varying amounts of the peptidodendrimer conjugate(s) of the invention are administered to cells in culture and the concentration effective for obtaining the desired result is calculated. Determination of effective amounts for in vivo administration may also involve in vitro assays in which varying doses of agent are administered to cells in culture and the concentration of agent effective for achieving the desired result is determined in order to calculate the concentration required in vivo. Effective amounts may also be based on in vivo animal studies.

The present invention may be further illustrated by reference to the following examples.

### EXAMPLES

The following Examples are intended to illustrate, but by no means are intended to limit, the scope of the present invention as set forth in the appended claims.

### Example 1

### Synthesis of Monofunctional Dendrimers

Monofunctional dendrimers were synthesized as shown in Scheme 1 below and as described in Tarallo et al., “Dendrimers Functionalized with Membrane-Interacting Peptides for Viral Inhibition,” Int'l J. Nanomedicine 8:521-34 (2013), which is hereby incorporated by reference in its entirety.

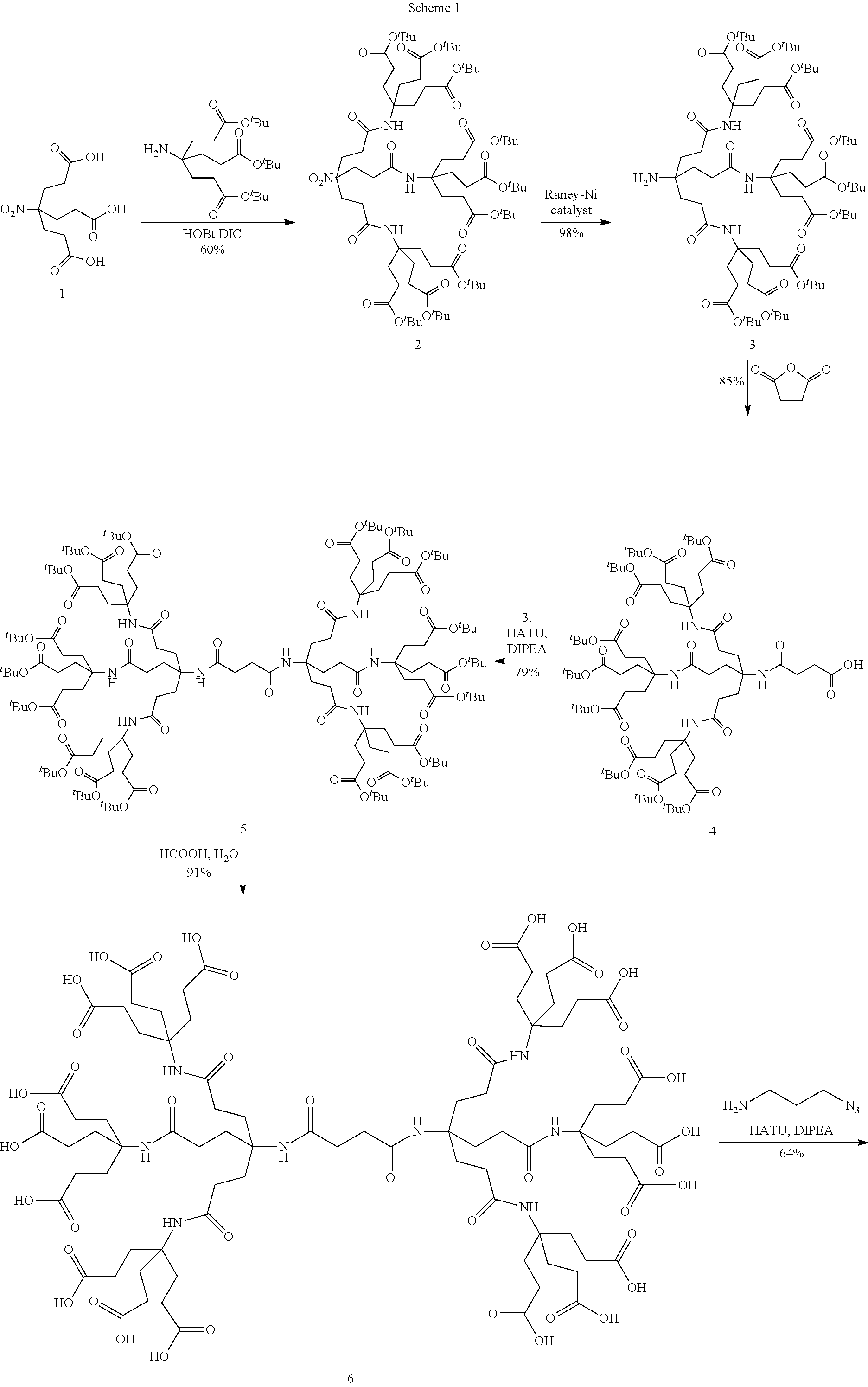

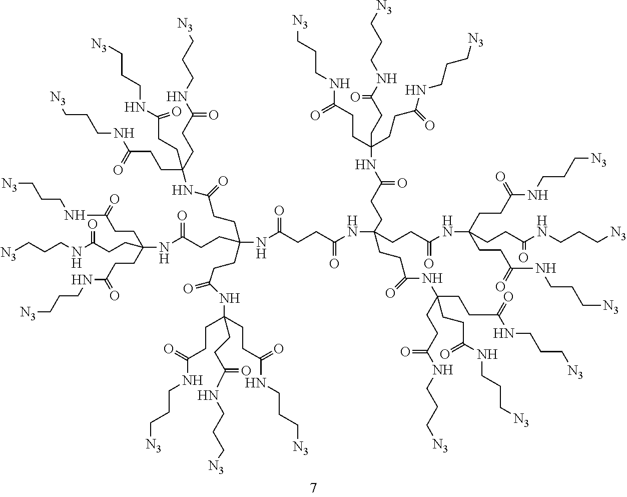 [SMILES: [N-]=[N+]=NCCCNC(=O)CCC(CCC(=O)NCCCN=[N+]=[N-])(CCC(=O)NCCCN=[N+]=[N-])NC(=O)CCC(CCC(=O)NC(CCC(=O)NCCCN=[N+]=[N-])(CCC(=O)NCCCN=[N+]=[N-])CCC(=O)NCCCN=[N+]=[N-])(CCC(=O)NC(CCC(=O)NCCCN=[N+]=[N-])(CCC(=O)NCCCN=[N+]=[N-])CCC(=O)NCCCN=[N+]=[N-])NC(=O)CCC(=O)NC(CCC(=O)NC(CCC(=O)NCCCN=[N+]=[N-])(CCC(=O)NCCCN=[N+]=[N-])CCC(=O)NCCCN=[N+]=[N-])(CCC(=O)NC(CCC(=O)NCCCN=[N+]=[N-])(CCC(=O)NCCCN=[N+]=[N-])CCC(=O)NCCCN=[N+]=[N-])CCC(=O)NC(CCC(=O)NCCCN=[N+]=[N-])(CCC(=O)NCCCN=[N+]=[N-])CCC(=O)NCCCN=[N+]=[N-]]

### Example 2

### Synthesis of Monofunctional Peptidodendrimer Conjugates

Monofunctional dendrimer 7 was conjugated with HSV-1 envelope glycoprotein peptides to form monofunctional peptidodendrimer conjugates using standard click chemistry, as illustrated in Scheme 2 below.

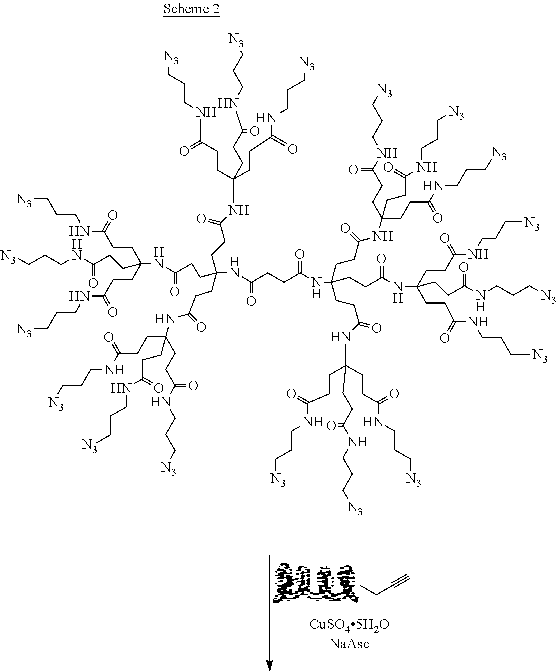

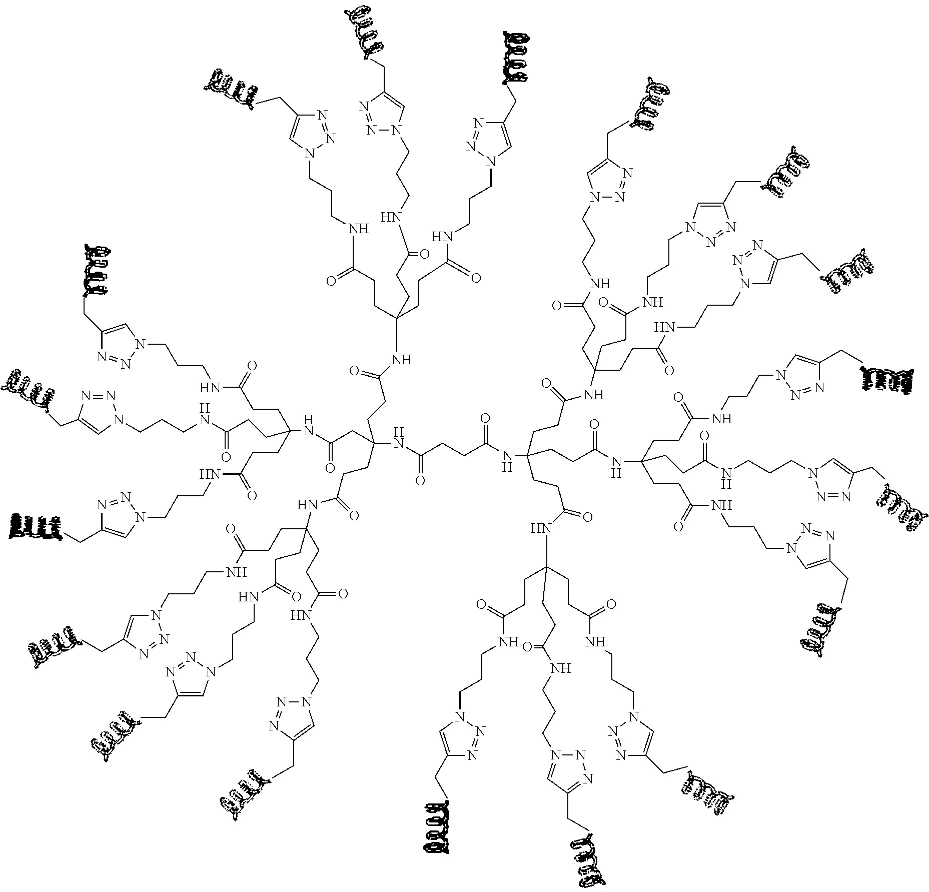

In particular, dendrimer 7 was conjugated with the HSV-1 envelope glycoprotein peptides shown in Table 3 below.

TABLE 3
HSV-1 Envelope Glycoprotein-Derived Peptides

| Name | Description | Sequence |
|---|---|---|
| gB8 or | Residues 503-523 of envelope | FARLQFTYNHIQRHVRDMEGR |
| gB503-523 | glycoprotein B | (SEQ ID NO: 23) |
| PgH | Residues 493-512 of envelope | AAHLIDALYAEFLGGRVLTT |
| | glycoprotein H | (SEQ ID NO: 53) |
| gC1 | Residues 137-151 of envelope | GSRVQIRCRFRNSTR |
| | glycoprotein C | (SEQ ID NO: 43) |
| g1 | Selected as anti-heparin sulfate | LRSRTKIIRIRH |
| | by peptide phage library | (SEQ ID NO: 61) |
| g2 | Selected as anti-heparin sulfate | MPRRRRIRRRQK |
| | by peptide phage library | (SEQ ID NO: 63) |

The formation of a monofunctional peptidodendrimer conjugated to peptide gB503-523 is described below by way of example.

The peptide sequence was synthesized with a propargylglycine residue (PrA) at the N-terminus to provide a handle for the copper-catalyzed azide/alkyne cycloaddition reaction (CuAAC). Functionalization of monofunctional dendrimer (1 equivalent) with Pra-gB503-523 (36 equivalents, 2.68 mg, 9.64e10-3 mmol) was performed in a water/methanol solution (1:1 v/v, about 1 ml) by using 2:4 equivalents (to the azide moiety) of CuSO₄.5H₂O:sodium ascorbate. The reaction was left stirring for 1 hour at 40° C. and for 2 days at room temperature. The compound was dialyzed against water/EDTA with 1000 MWCO membranes over night. The peptidodendrimer conjugate was purified by reverse phase HPLC on C4 column with water (0.1% TFA) and acetonitrile (0.1% TFA) from 5 to 90% Acn over 20 min at 5 ml/min flow. (See FIGS. 4A-4D).

Following HPLC purification, the peptidodendrimer conjugate was passed on 30 KDa (MWCO) ultrafiltration membranes for three times using water:MeOH:DMSO 50/45/5. From the ultrafiltration the functionalization degree was found to be of at least 55% (at least 10 copies of the peptide are attached on the dendrimer).

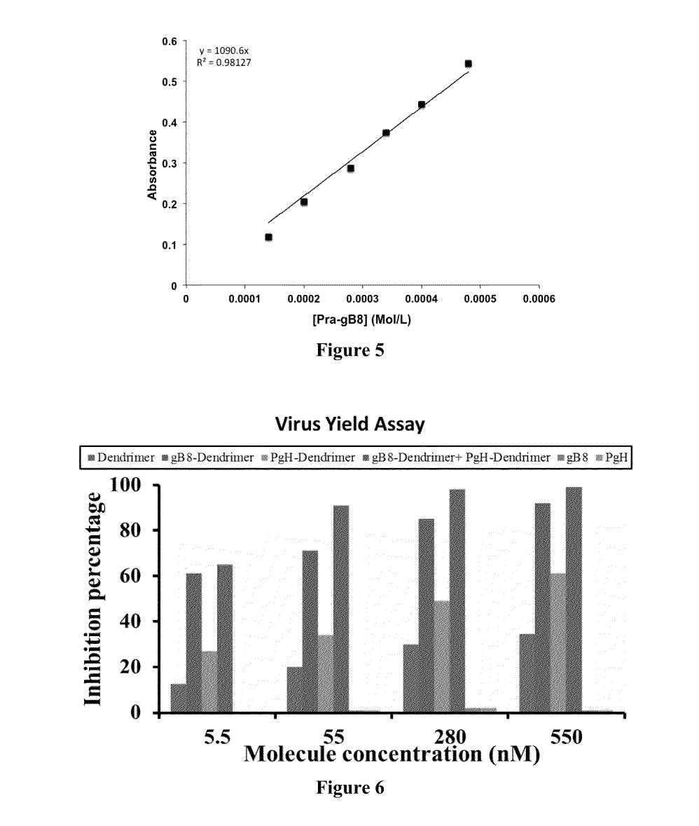

The reaction yield was confirmed by determining the amount of peptide attached by UV analysis (εgB=7000 m⁻¹ cm⁻¹ at λ=280 nm) (FIG. 5) and comparing this to the amount of peptide initially used for reaction (36 mol peptide per mol dendrimer). In particular the UV linear calibration curve was developed by measuring the absorbance of the aromatic residues (two phenylalanines (F) and one tyrosine (Y)) present in the peptide sequence at the wavelength of 280 nm at different Pra-gB8 concentrations and in water:acetonitrile solution (75:15). The absorption spectra were recorded at room temperature on an Agilent 8453 UV-visible single beam spectrophotometer with 10 mm pathlength quartz cuvettes. The reaction yield was found to be 62%.

### Example 3

### Antiviral Studies: General Materials and Methods

### Cells and Viruses

African green monkey kidney cells (Vero) (ATCC CCL-81) were grown in Dulbecco's modified Eagle's medium (DMEM) supplemented with 10% fetal calf serum. HSV-1 (strain SC16), carrying a lacZ gene driven by the CMV IE-1 promoter to express β-galactosidase, was propagated on Vero cells monolayers.

### Virus Entry Assays

To assess the effect of peptides on inhibition of HSV infectivity, cell monolayers were evaluated as described in Examples 4-10 below. For all experiments, peptides, dendrimers, and peptidodendrimers were dissolved in DMEM without serum and used at concentrations of 0, 5.5, 55, 280, and 550 nM. All experiments were conducted in triplicate. The percentage of infectivity inhibition was calculated by setting the number of plaques obtained in positive controls where no antiviral compounds were added to the cell monolayers to 0% inhibition.

### Peptides, Dendrimers, and Peptidodendrimer Conjugates

The following peptides, dendrimers, and peptidoconjugates were used in Examples 4-8. Monofunctional dendrimer 7 alone (“Dendrimer”), peptide gB8 alone (“gB8”), peptide PgH alone (“PgH”), monofunctional dendrimer conjugated with peptide gB8 (“gB8-Dendrimer”), monofunctional dendrimer conjugated with peptide PgH (“PgH-Dendrimer”), or a 1:1 mixture of gB8-Dendrimer and PgH-Dendrimer (“gB8-Dendrimer+PgH-Dendrimer”) (the total peptidodendrimer concentration was conserved).

### Example 4

### Virus Yield Reduction Assay Using Monofunctional Peptidodendrimer Conjugates

Confluent Vero cell monolayers (12-well plates) were washed with phosphate-buffered saline (PBS) and infected with HSV-1 at multiplicity of infection (MOI) of 1 plaque-forming unit (pfu)/cell for 1 hour at 37° C. The virus inocula were mixed with the peptide/dendrimer/conjugate(s) to be tested, as described in Example 3 above. Nonpenetrated viruses were inactivated by citrate buffer at pH 3.0. The infected cells were washed with PBS, covered with fresh culture medium, and incubated for 48 hours. The infected cells were then scraped into culture medium and disrupted by sonication. The total virus yield in each well was titrated by plaque assay. Plaques were stained with X-gal (5-bromo-4-chloro-3-indolyl-(3-D-galactopyranoside) and microscopically counted. The mean plaque counts for each drug concentration were expressed as a percentage of the mean plaque count for the control virus. The number of plaques was plotted as a function of drug concentration. See FIG. 6.

### Example 5

### Co-Treatment Assay

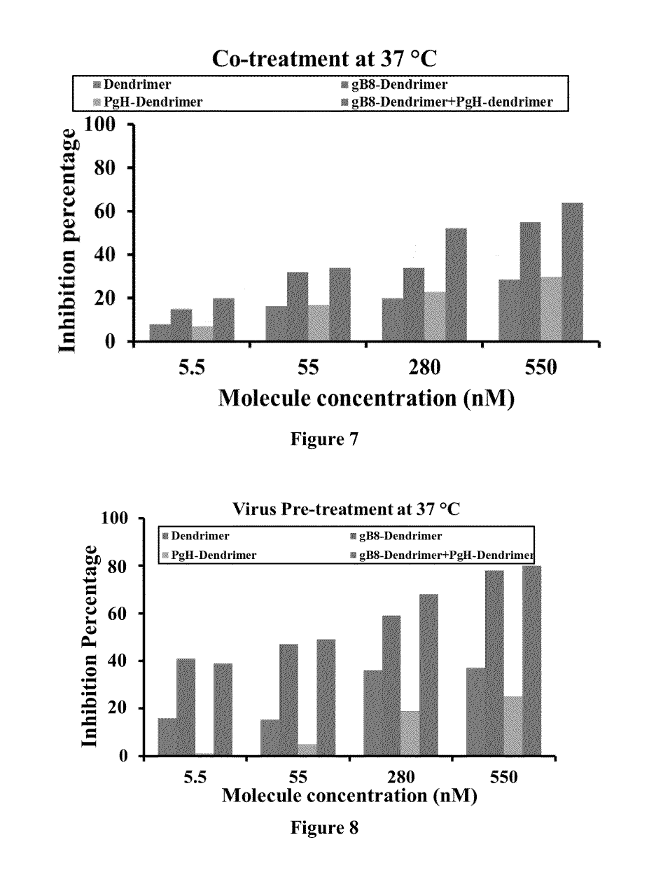

Confluent Vero cell monolayers (12-well plates) were washed with phosphate-buffered saline and infected with HSV-1 at a multiplicity of infection of 0.02 plaque-forming units per cell for 1 hour at 37° C. The virus inocula were mixed with the dendrimer/conjugate(s) to be tested. Nonpenetrated viruses were inactivated by citrate buffer at pH 3.0. The infected cells were washed with phosphate-buffered saline, overlaid with fresh culture medium supplemented with carboxymethyl cellulose, and incubated for 48 hours. Monolayers infected with HSV-1 were fixed and stained with X-gal. Plaques were counted microscopically. The mean plaque counts for each drug concentration were expressed as a percentage of the mean plaque count for the control virus. The number of plaques was plotted as a function of drug concentration. See FIG. 7.

### Example 6

### Virus Pre-Treatment Assay

The dendrimer/conjugate(s) to be tested were added to aliquots of HSV-1 (10⁴ pfu) and incubated at 37° C. for 2 hours. After incubation, the samples were diluted with medium to reduce the concentration of the antiviral compound to one that was not active in an antiviral assay. The MOI of HSV-1 after dilution was of 0.01 pfu/cell. The viruses were then titrated on Vero cell monolayers. Plates were then fixed, stained with X-gal, and the number of plaques was scored. See FIG. 8.

### Example 7

### Cell Pre-Treatment Assay

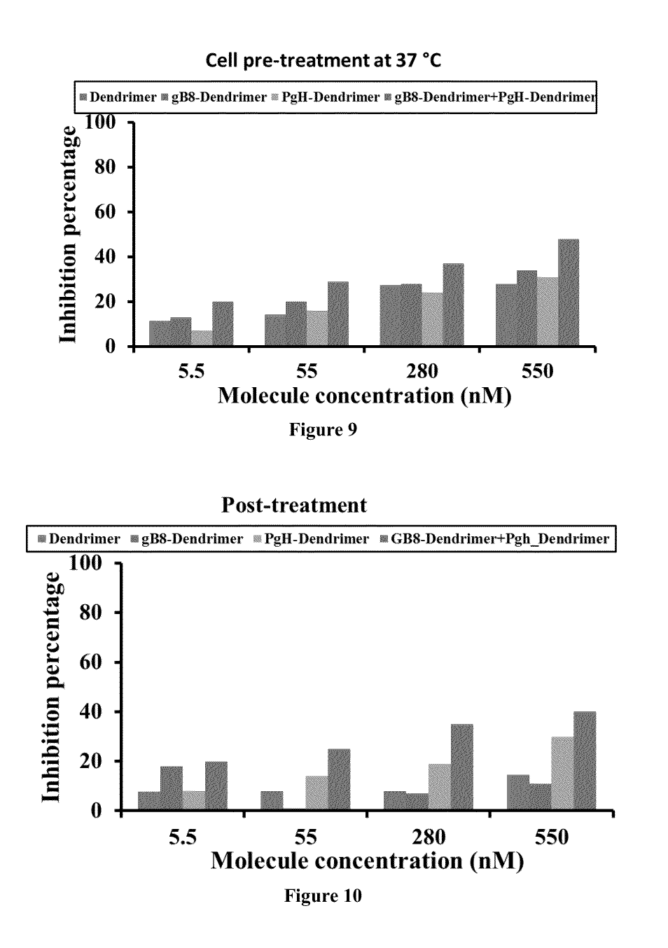

Confluent Vero cell monolayers (12 well-plates) were treated with the dendrimer/conjugate(s) to be tested for 2 hours at 4° C. or at 37° C. and then infected with HSV-1 at an MOI of 0.1 pfu/cell. The cells were then washed three times with Dulbecco's Modified Eagle's Medium to remove unattached virus and nanoparticles, overlaid with carboxymethyl cellulose, and incubated for 2 days at 37° C. After fixing, plates were fixed and stained with X-gal and the number of plaques was scored. See FIG. 9.

### Example 8

### Post-Treatment Assay

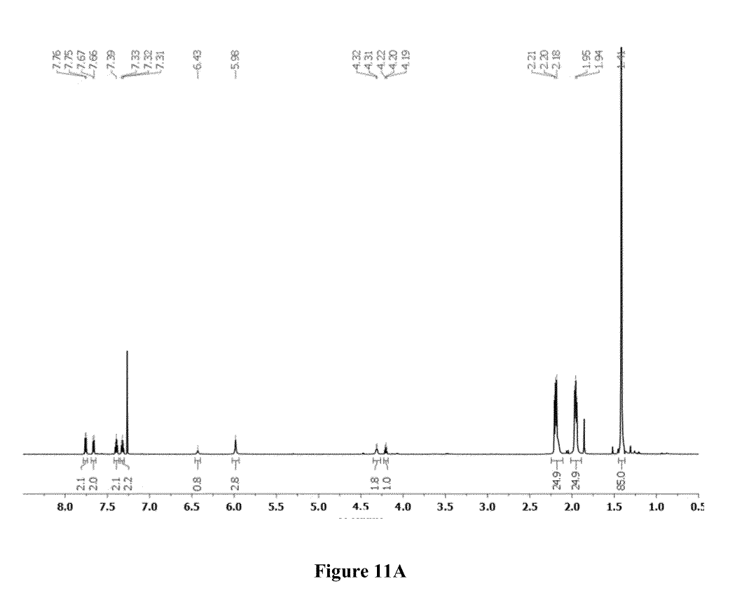

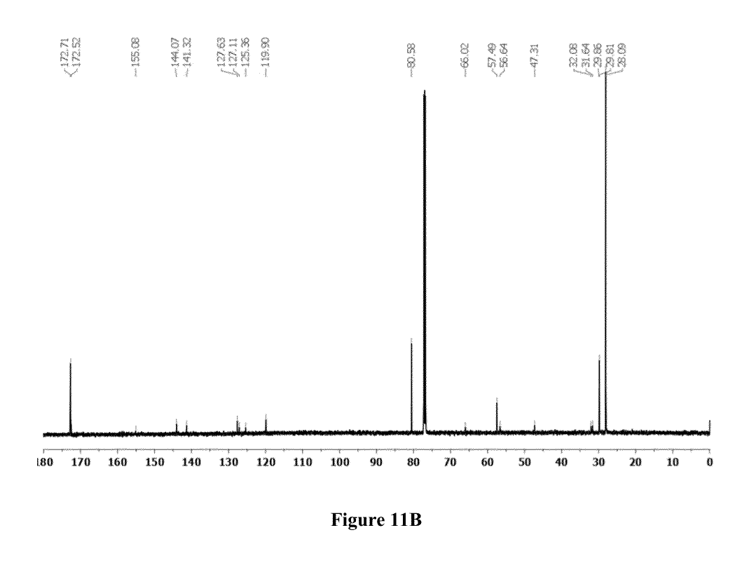

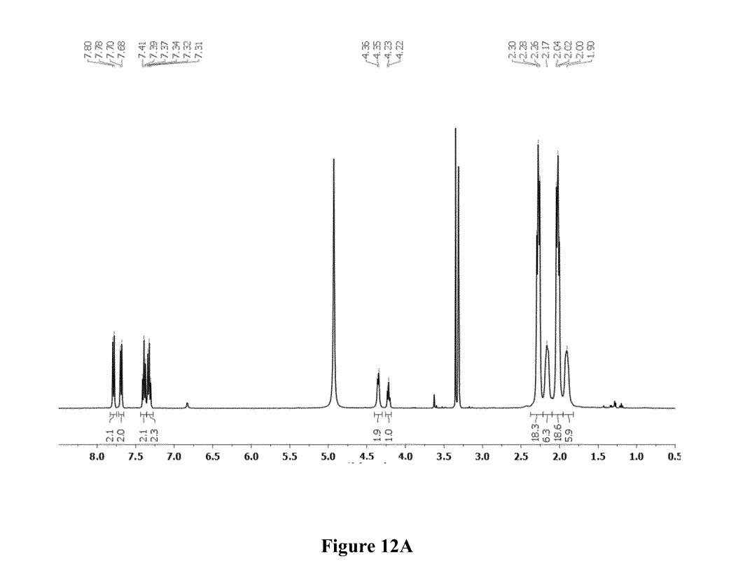

Vero cell monolayers (12-well plates) were incubated with HSV-1 for 45 minutes at 37° C. The dendrimer/conjugate(s) to be tested were then added to the inoculum followed by an additional incubation period of 30 minutes at 37° C. For all treatments, nonpenetrated viruses were inactivated by citrate buffer at pH 3.0 after the 45 minute incubation with cells at 37° C. The cells were then incubated for 24 hours at 37° C. in DMEM supplemented with carboxymethyl cellulose (CMC). Monolayers were fixed, stained with X-gal, and plaque numbers were scored. See FIG. 10.

### Discussion of Examples 1-8

The gB8-Dendrimer was shown to be very active and conjugating peptide gB8 to the dendrimer was shown to significantly reduce the inhibitory concentration of the peptide (from the micromolar to the nanomolar range). The gB8-Dendrimer was also found, surprisingly, to have significantly higher antiviral activity than the previously-described monofunctional peptidodendrimer conjugated with gH625-644 (which was found to have an IC₅₀ of 100 nM and 300 nM against, respectively, HSV-1 and HSV-2). The PgH-Dendrimer was also more effective than the peptide alone or the dendrimer alone. This demonstrates that conjugating envelope glycoprotein-derived peptides to dendrimers can enhance their efficacy.

The gB8-Dendrimer was also shown to work very well when added together with the virus. This supports the view that gB8 interacts with the virus. All the other antiviral data confirm this result.

Using a mixture of gB8-Dendrimer and PgH-Dendrimer improved the inhibition activity relative to either conjugate alone. This demonstrates that using a mixture of dendrimers conjugated with peptides that have different targets is a good strategy for further improving the inhibitory activity (and thereby reducing the inhibitory concentration). It is expected that an even higher increase can be achieved using another peptide in place of PgH, which is less active than gB8.

### Example 9

### Synthesis of Bifunctional Dendrimers

Bifunctional dendrimers were synthesized as shown in Scheme 3 below (see Newkome et al., J. Org. Chem. 56:7162-67 (1991); Brettreich & Hirsch, Synlett 1396-98 (1998); Carberry et al., Chem. Eur. J. 18:13678-85 (2012); Vercillo et al., Org. Lett. 10:205-08 (2008), each of which is hereby incorporated by reference in its entirety).

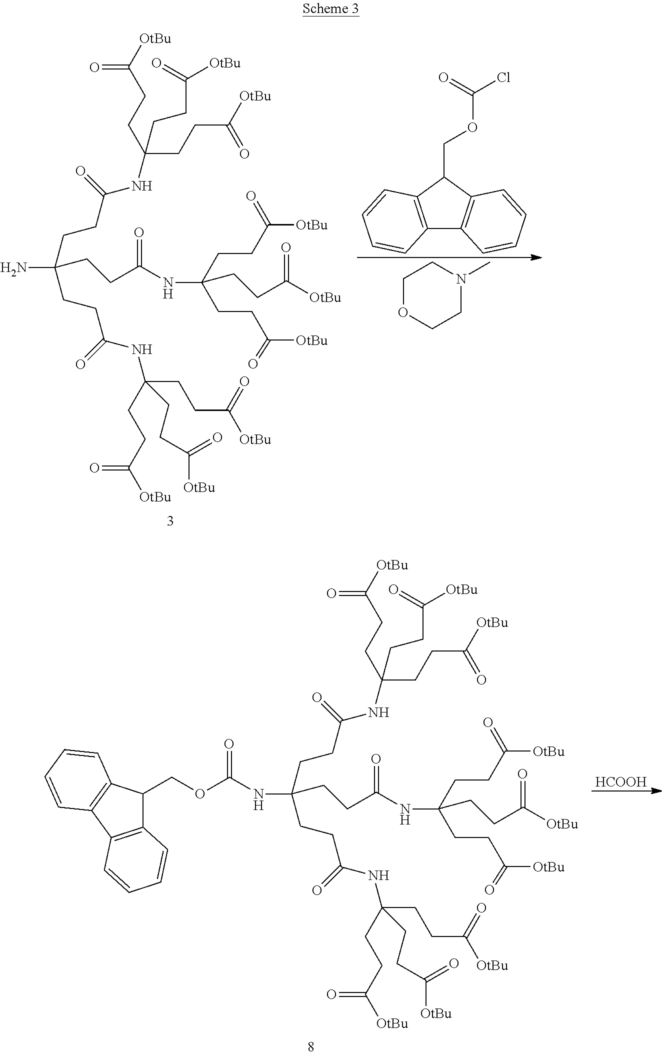

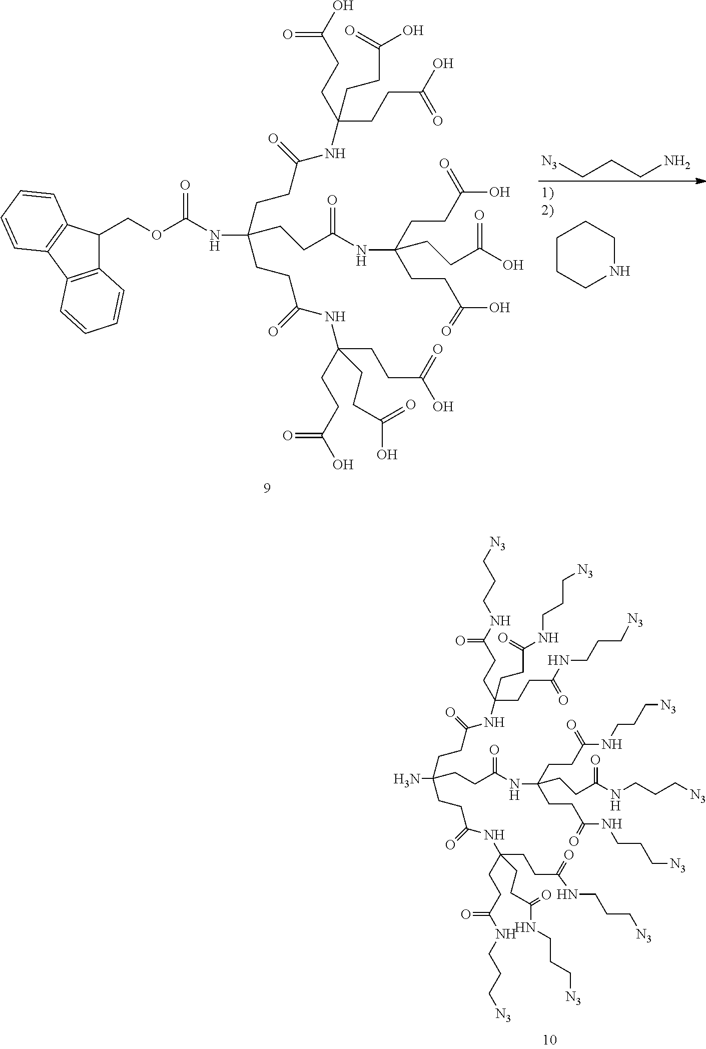 [SMILES: O=C(O)CCC(CCC(=O)O)(CCC(=O)O)NC(=O)CCC(CCC(=O)NC(CCC(=O)O)(CCC(=O)O)CCC(=O)O)(CCC(=O)NC(CCC(=O)O)(CCC(=O)O)CCC(=O)O)NC(=O)OCC1c2ccccc2-c2ccccc21 ; [N-]=[N+]=NCCCN ; C1CCNCC1 ; [N-]=[N+]=NCCCNC(=O)CCC(CCC(=O)NCCCN=[N+]=[N-])(CCC(=O)NCCCN=[N+]=[N-])NC(=O)CCC(N)(CCC(=O)NC(CCC(=O)NCCCN=[N+]=[N-])(CCC(=O)NCCCN=[N+]=[N-])CCC(=O)NCCCN=[N+]=[N-])CCC(=O)NC(CCC(=O)NCCCN=[N+]=[N-])(CCC(=O)NCCCN=[N+]=[N-])CCC(=O)NCCCN=[N+]=[N-]]

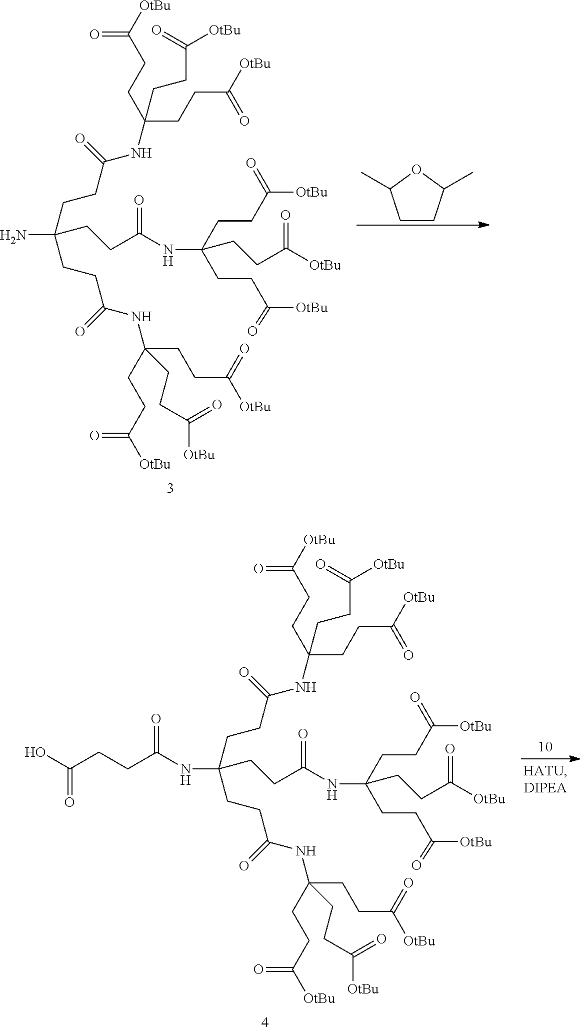

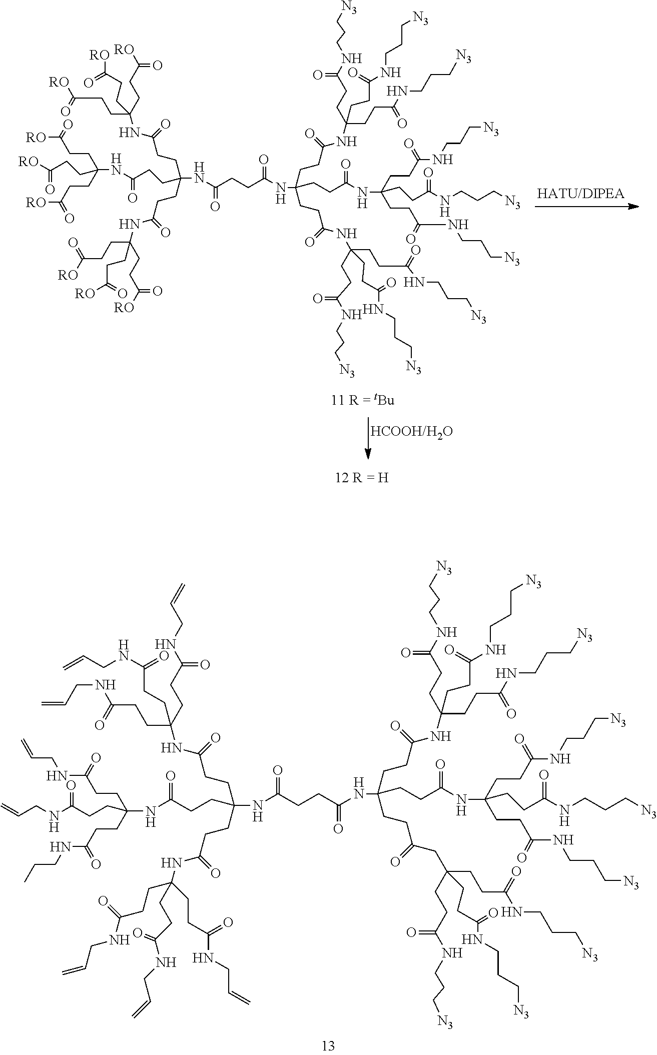

### Synthesis of Dendron 8

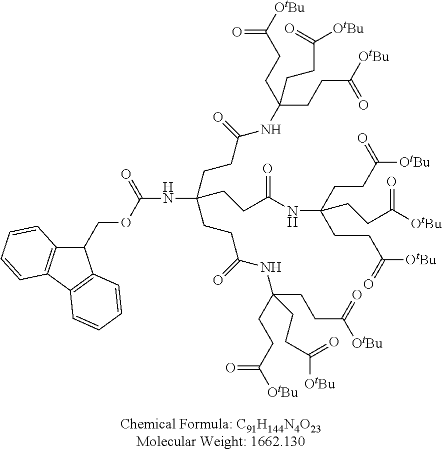 [SMILES: CC(C)(C)OC(=O)CCC(CCC(=O)OC(C)(C)C)(CCC(=O)OC(C)(C)C)NC(=O)CCC(CCC(=O)NC(CCC(=O)OC(C)(C)C)(CCC(=O)OC(C)(C)C)CCC(=O)OC(C)(C)C)(CCC(=O)NC(CCC(=O)OC(C)(C)C)(CCC(=O)OC(C)(C)C)CCC(=O)OC(C)(C)C)NC(=O)OCC1c2ccccc2-c2ccccc21]

Dendron 3 (see Example 1 above) (0.500 g, 347.25 μmol) and 9-fluorenylmethylchloroformate (0.259 g, 1.00 mmol) were placed in a Schlenk flask and the atmosphere was replaced with nitrogen. THF (10 mL) was added and the reaction was cooled in an ice bath. N-methylmorpholine (0.80 mL, 694.80 μmol) was added dropwise, and the reaction was left to stir as the ice melted for 1 day. The reaction mixture was then diluted with EtOAc and washed with KHSO₄ (99 mL H₂O, 0.95 mL H₂SO₄, 0.966 g KOH), water, and brine. The organic layer was dried over sodium sulfate and filtered. The product was purified by silica gel column chromatography (hexane→3:1 hexane:EtOAc→1:1) to yield product 8 as a white foam (0.480 g, 83%). ¹HNMR (FIG. 11A) (600 MHz, CDCl₃, δₚₚₘ vs. TMS): 7.76 (d, J=7.5 Hz, 2H, ArH); 7.67 (d, J=7.5 Hz, 2H, ArH); 7.39 (t, J=7.4 Hz, 2H, ArH); 7.32 (t, J=7.4 Hz, 2H, ArH); 6.43 (br s, 1H, OCONH); 5.98 (br s, 3H, CH₂CONH); 4.32 (br d, J=6.7 Hz, 2H, CH₂OCON); 4.20 (t, J=7.5 Hz, 1H, CHAr₂); 2.20 (app t, J=8.0 Hz, 24H, CH₂C00); 1.95 (app t, J=8.0 Hz, 24H, C_{q}CH₂CH₂); 1.41 (s, 81H, C(CH₃)₃). ¹³CNMR (FIG. 11B) (150 MHz, CDCl₃, δₚₚₘ vs. TMS): 172.71; 172.52; 155.08; 144.07; 141.32; 127.63; 119.90; 80.58; 66.02; 57.49; 56.64; 47.31; 32.08; 31.64; 29.86; 29.81; 28.09. MS-ESI (M+Na)⁺ m/z calcd for C₉₁H₁₄₄N₄O₂₃Na: 1684.012. found 1684.1.

### Synthesis of Dendron 9

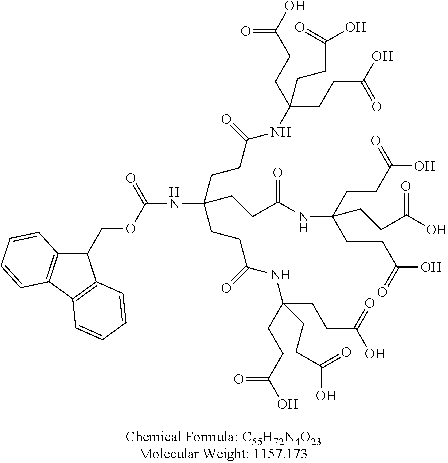 [SMILES: O=C(O)CCC(CCC(=O)O)(CCC(=O)O)NC(=O)CCC(CCC(=O)NC(CCC(=O)O)(CCC(=O)O)CCC(=O)O)(CCC(=O)NC(CCC(=O)O)(CCC(=O)O)CCC(=O)O)NC(=O)OCC1c2ccccc2-c2ccccc21]

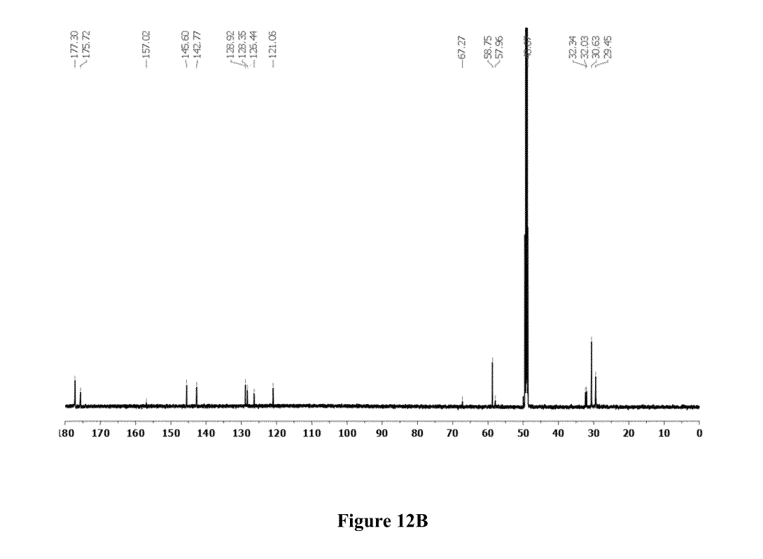

Dendron 8 (0.465 g, 279.76 μmol) was placed in a roundbottom flask and dissolved in a formic acid:water (40 mL:4 mL) mixture. This was left to stir for 8 hours at room temperature. The solvent was removed and the product was precipitated from Et₂O. After centrifugation, the insoluble product was collected and dried under vacuum to afford 9 as a white solid (0.311 g, 96%). ¹HNMR (FIG. 12A) (400 MHz, MeOD, δₚₚₘ vs. MeOD): 7.79 (d, J=7.5 Hz, 2H, ArH); 7.69 (d, J=7.4 Hz, 2H, ArH); 7.39 (t, J=7.4 Hz, 2H, ArH); 7.32 (t, J=7.1 Hz, 2H, ArH); 4.36 (br d, J=6.5 Hz, 2H, CH₂OCON); 4.23 (t, J=6.6 Hz, 1H, CHAr₂); 2.37-2.21 (m, 18H, CONC_{q}CH₂CH₂); 2.21-2.09 (m, 6H, FmocNHC_{q}CH₂CH₂); 2.09-1.95 (m, 18H, CONC_{q}CH₂); 1.95-1.81 (m, 6H, FmocNHC_{q}CH₂). ¹³CNMR (FIG. 12B) (150 MHz, MeOD, δₚₚₘ vs. MeOD): 177.3; 175.7; 157.0; 145.6; 142.8; 128.9; 128.3; 126.4; 121.1; 67.3; 58.8; 57.9; 48.7; 32.3; 32.0; 30.6; 29.5. MS-ESI (M−H)⁻ m/z calcd for C₅₅H₇₁N₄O₂₃: 1155.451. found 1155.6.

### Synthesis of Dendron 10

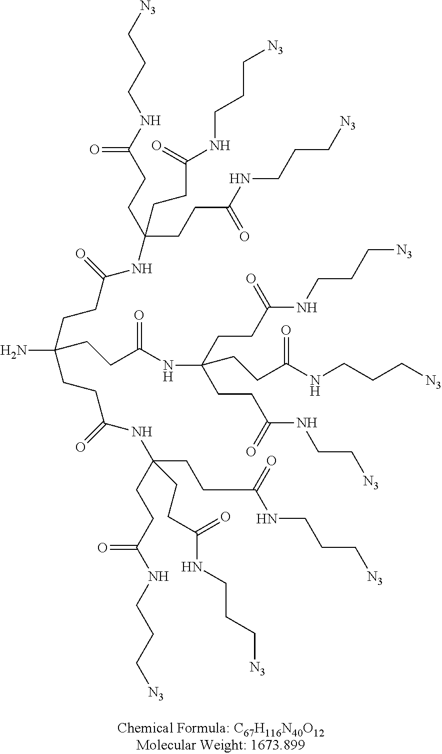 [SMILES: [N-]=[N+]=NCCCNC(=O)CCC(CCC(=O)NCCCN=[N+]=[N-])(CCC(=O)NCCCN=[N+]=[N-])NC(=O)CCC(N)(CCC(=O)NC(CCC(=O)NCCCN=[N+]=[N-])(CCC(=O)NCCCN=[N+]=[N-])CCC(=O)NCCCN=[N+]=[N-])CCC(=O)NC(CCC(=O)NCCCN=[N+]=[N-])(CCC(=O)NCCCN=[N+]=[N-])CCC(=O)NCCN=[N+]=[N-]]

To a solution of dendron 9 (0.134 g, 115.80 μmol) and HATU (0.440 g, 1.16 mmol) in DMF (3.3 mL) was added DIPEA (0.404 mL, 2.32 mmol) and 3-azidopropylamine (0.334 g, 2.89 mmol). The whole was stirred for one day until LCMS analysis showed the formation of the product. MS-ESI (M+H)⁺ m/z calcd for C₈₂H₁₂₇N₄₀O₁₄: 1897.145. found 1897.1. This solution was used directly without further purification for the synthesis of dendron 10.

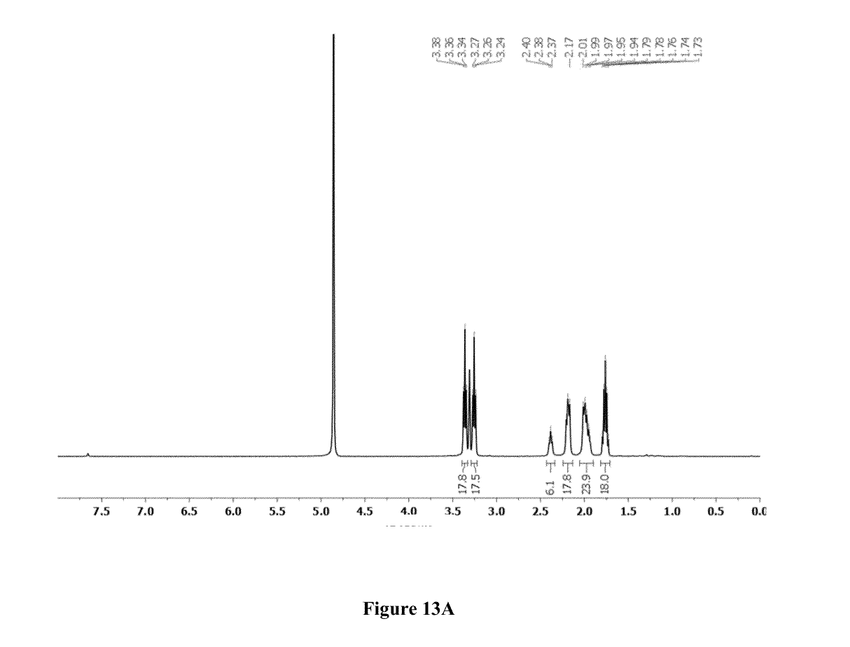

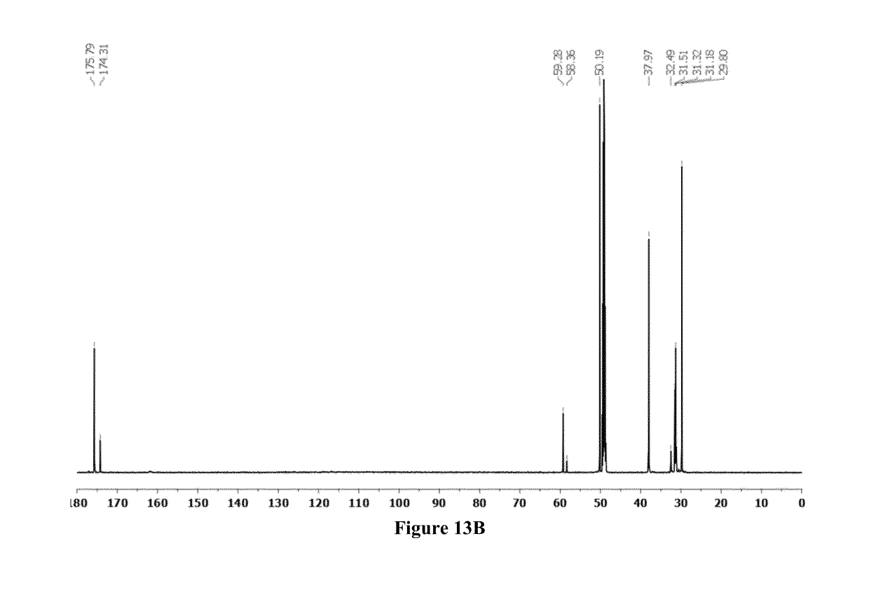

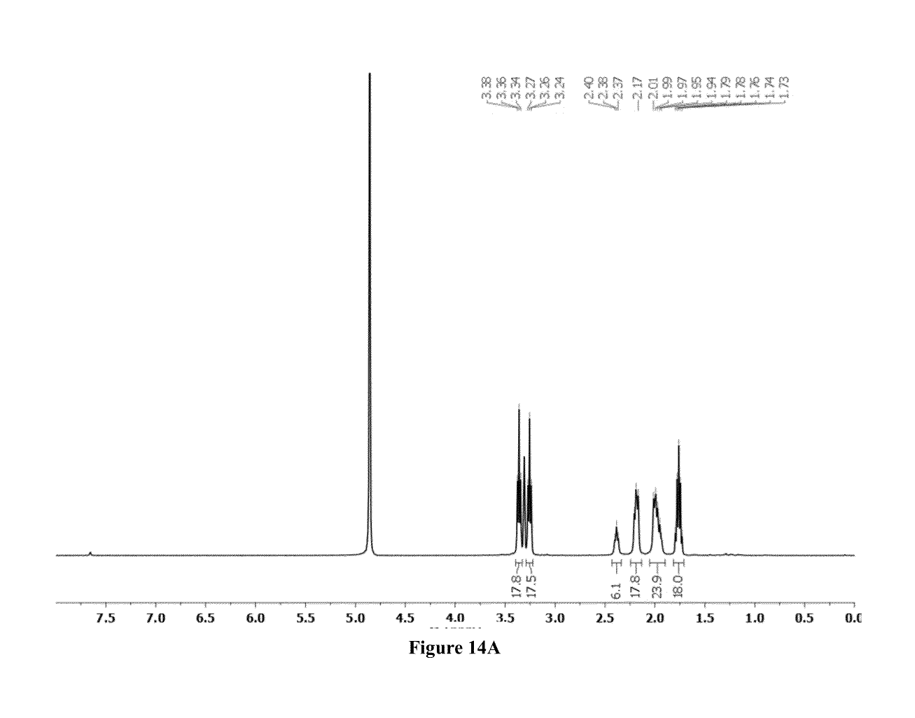

Piperidine (1 mL) was added to the solution and stirring was continued for 3 hours. After removal of solvent, the crude product was precipitated from Et₂O to afford an insoluble yellow oil. The oil was purified by semi-preparative HPLC (25-80% ACN in water over 20 min, retention time 17.7 min). Dendron 10 was obtained as a yellowish glass (0.078 g, 40% after 2 steps and purification). ¹HNMR (FIG. 13A) (400 MHz, MeOD, δₚₚₘ vs. MeOD): 3.36 (t, J=6.6 Hz, 18H, CONHCH₂); 3.26 (t, J=6.7 Hz, 18H, CH₂N₃); 2.38 (t, J=6.9 Hz, 6H, CH₂CONHC_{q}); 2.19 (app t, J=7.9 Hz, 18H, CH₂CO); 2.05-1.89 (m, 24H, C_{q}CH₂); 1.76 (p, J=6.7 Hz, 18H, CH₂CH₂CH₂N₃). ¹³CNMR (FIG. 13B) (150 MHz, MeOD, δₚₚₘ vs. MeOD): 175.79; 174.31; 59.78; 58.36; 50.19; 37.97; 32.49; 31.51; 31.32; 31.18; 29.80. MS-ESI (M+H)⁺ m/z calcd for C₆₇H₁₁₇N₄₀O₁₂: 1673.977. found 1673.2.

### Synthesis of Dendrimer 11

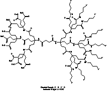 [SMILES: CC(C)(C)OC(=O)CCC(CCC(=O)OC(C)(C)C)(CCC(=O)OC(C)(C)C)NC(=O)CCC(CCC(=O)NC(CCC(=O)OC(C)(C)C)(CCC(=O)OC(C)(C)C)CCC(=O)OC(C)(C)C)(CCC(=O)NC(CCC(=O)OC(C)(C)C)(CCC(=O)OC(C)(C)C)CCC(=O)OC(C)(C)C)NC(=O)CCC(=O)NC(CCC(=O)NC(CCC(=O)NCCCN=[N+]=[N-])(CCC(=O)NCCCN=[N+]=[N-])CCC(=O)NCCCN=[N+]=[N-])(CCC(=O)NC(CCC(=O)NCCCN=[N+]=[N-])(CCC(=O)NCCCN=[N+]=[N-])CCC(=O)NCCCN=[N+]=[N-])CCC(=O)NC(CCC(=O)NCCCN=[N+]=[N-])(CCC(=O)NCCCN=[N+]=[N-])CCC(=O)NCCCN=[N+]=[N-]]

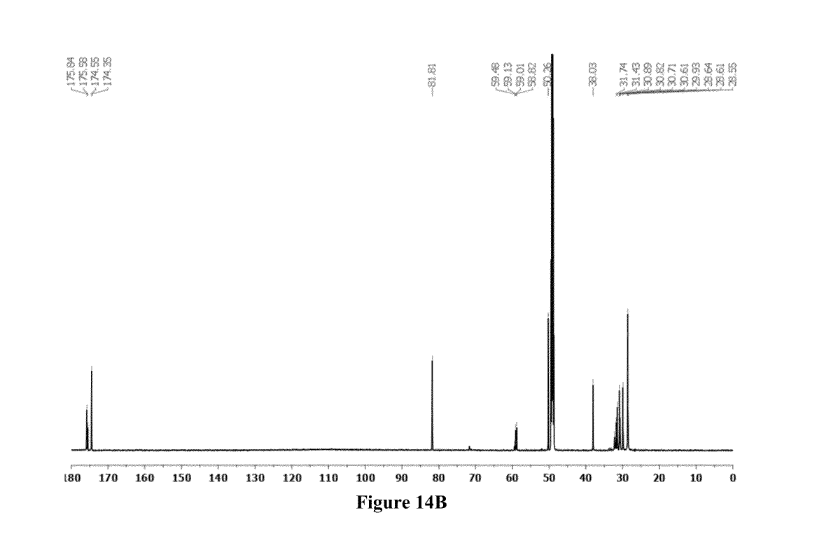

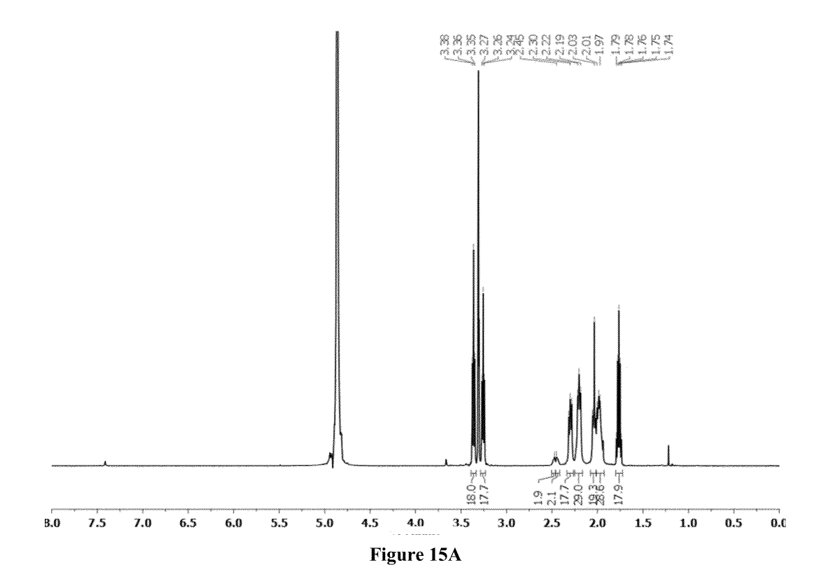

A Schlenk flask was charged with dendron 10 (0.107 g, 63.9 μmol) and dendron 4 (see Example 1 above) (0.079 g, 51.1 μmol), HATU (0.025 g, 66.5 μmol), DMF (3.5 mL), and DIPEA (23 μL, 127.8 μmol) under an inert atmosphere. After stirring at room temperature for 2 days, the solvent was removed in vacuo and the residue was purified by semi-preparative HPLC (40-85% ACN in water over 5 min then 85-100% over 15 min, retention time 12.5 min). Dendrimer 11 was obtained as a colorless to slightly yellow glass (0.114 g, 70%). ¹HNMR (FIG. 14A) (400 MHz, MeOD, δₚₚₘ vs. MeOD): 3.37 (t, J=6.2 Hz, 18H, CONHCH₂); 3.26 (t, J=6.4 Hz, 18H, CH₂N₃); 2.47 (br s, 4H, COCH₂CH₂CO); 2.27-2.11 (m, 48H, CH₂CO); 2.08-1.86 (m, 48H, C_{q}CH₂); 1.77 (p, J=6.3 Hz, 18H, CH₂CH₂CH₂N₃); 1.45 (s, 81H, C(CH₃)₃). ¹³CNMR (FIG. 14B) (150 MHz, MeOD, δₚₚₘ vs. MeOD): 175.8; 175.6; 174.6; 174.4; 81.8; 59.5; 59.1; 59.0; 58.8; 50.2; 38.0; 31.7; 31.4; 30.9; 30.8; 30.7; 30.6; 29.9; 28.7. 28.6; 28.5. MS-ESI (M+3H)⁺³ m/z calcd for C₁₄₇H₂₅₅N₄₄O₃₅: 3196.953/3=1065.651. found 1066.1; MS-ESI (M+H+3Na)⁺⁴ m/z calcd for C₁₄₇H₂₅₃N₄₄O₃₅Na₃: 3263.906/4=815.977. found 815.3.

### Synthesis of Dendrimer 12

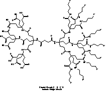 [SMILES: [N-]=[N+]=NCCCNC(=O)CCC(CCC(=O)NCCCN=[N+]=[N-])(CCC(=O)NCCCN=[N+]=[N-])NC(=O)CCC(CCC(=O)NC(CCC(=O)NCCCN=[N+]=[N-])(CCC(=O)NCCCN=[N+]=[N-])CCC(=O)NCCCN=[N+]=[N-])(CCC(=O)NC(CCC(=O)NCCCN=[N+]=[N-])(CCC(=O)NCCCN=[N+]=[N-])CCC(=O)NCCCN=[N+]=[N-])NC(=O)CCC(=O)NC(CCC(=O)NC(CCC(=O)O)(CCC(=O)O)CCC(=O)O)(CCC(=O)NC(CCC(=O)O)(CCC(=O)O)CCC(=O)O)CCC(=O)NC(CCC(=O)O)(CCC(=O)O)CCC(=O)O]

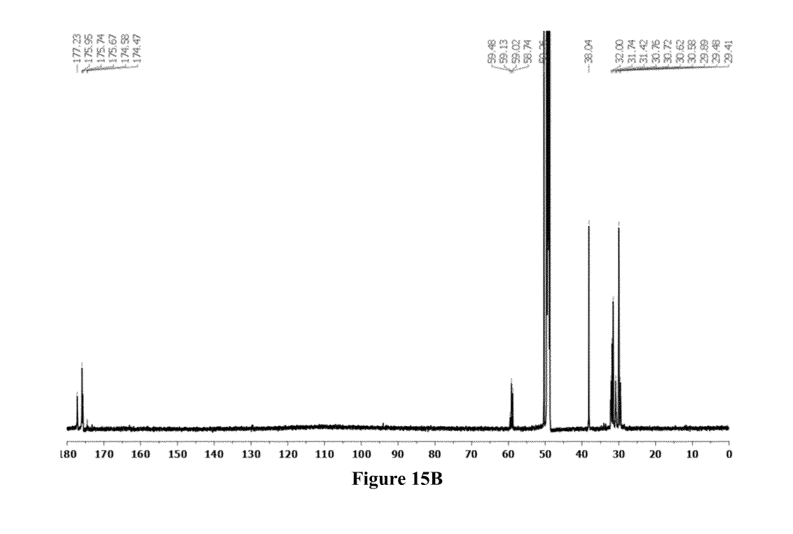

Dendrimer 11 (0.114 g, 35.67 μmol) was dissolved in formic acid and water (9 mL:0.9 mL) and left to stir 8 hours at room temperature. After removal of solvent, the product was precipitated from ether. Further purification was performed by HPLC (30-90% ACN in water over 20 min, retention time 16 min). Dendrimer 12 was obtained as a colorless to slightly yellow glass (0.073 g, 54% after purification). ¹HNMR (FIG. 15A) (500 MHz, MeOD, δₚₚₘ vs. MeOD): 3.36 (t, J=6.7 Hz, 18H, CONHCH₂); 3.26 (t, J=6.8 Hz, 18H, CH₂N₃); 2.51-2.46 (m, 2H, COCH₂CH₂CO); 2.46-2.41 (m, 2H, COCH₂CH₂CO); 2.30 (app t, J=8.5 Hz, 18H, CH₂CO); 2.20 (app t, J=8.1 Hz, 30H, CH₂CO); 2.03 (app t, J=8.1 Hz, 18H, C_{q}CH₂); 2.01-1.92 (m, 18H, C_{q}CH₂); 1.76 (p, J=6.8 Hz, 18H, CH₂CH₂CH₂N₃). ¹³CNMR (FIG. 15B) (150 MHz, MeOD, δₚₚₘ vs. MeOD): 177.2; 175.9; 175.7; 175.6; 174.6; 174.5; 59.5; 59.1; 59.0; 58.7; 50.3; 38.0; 32.0; 31.7; 31.4; 30.8; 30.7; 30.6; 30.5; 29.9; 29.5; 29.4.

### Synthesis of Dendrimer 13

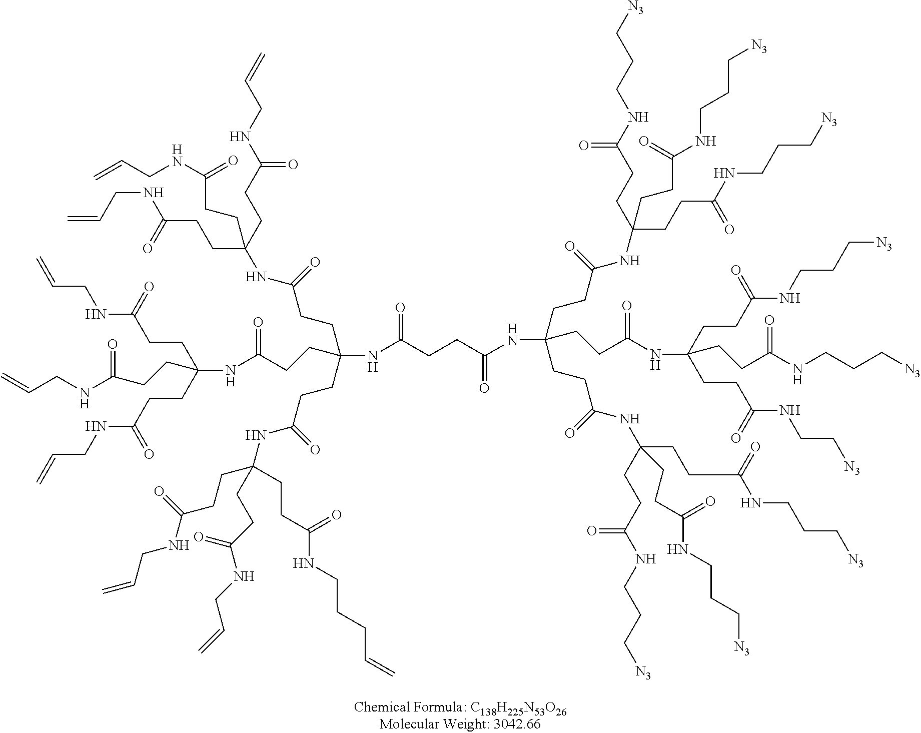 [SMILES: C=CCCCNC(=O)CCC(CCC(=O)NCC=C)(CCC(=O)NCC=C)NC(=O)CCC(CCC(=O)NC(CCC(=O)NCC=C)(CCC(=O)NCC=C)CCC(=O)NCC=C)(CCC(=O)NC(CCC(=O)NCC=C)(CCC(=O)NCC=C)CCC(=O)NCC=C)NC(=O)CCC(=O)NC(CCC(=O)NC(CCC(=O)NCCCN=[N+]=[N-])(CCC(=O)NCCCN=[N+]=[N-])CCC(=O)NCCCN=[N+]=[N-])(CCC(=O)NC(CCC(=O)NCCCN=[N+]=[N-])(CCC(=O)NCCCN=[N+]=[N-])CCC(=O)NCCCN=[N+]=[N-])CCC(=O)NC(CCC(=O)NCCCN=[N+]=[N-])(CCC(=O)NCCCN=[N+]=[N-])CCC(=O)NCCN=[N+]=[N-]]

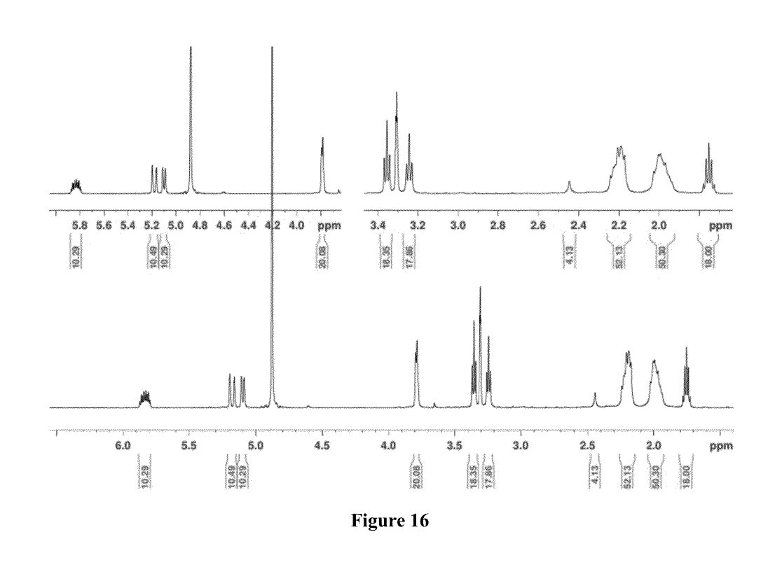

Dendrimer 13 (0.025 g; 9.29 μmol) was dissolved in 1:1 DMF-d₇:CDCl₂ (0.6 mL each) with HATU (0.064 g; 168.32 μmol) and allylamine (0.050 g; 875.81 μmol). After stirring for 5 minutes to ensure dissolution, DIPEA (70 μL, 383 μmol) was added, and the solution turned yellow. After 24 hours of mixing, the reaction was monitored daily via ¹H NMR Spectroscopy to determine conversion. When complete conversion was observed via NMR spectroscopy, the whole solution was diluted with methanol (ca. 15 mL) and transferred into a dialysis membrane (1000 MWCO). The product was dialyzed against methanol, changing the outer contents every 16 hours for three days. Concentration of the solution afforded Dendrimer 13 as a colorless oil (0.025 g; 88%). ¹HNMR (FIG. 16) (600 MHz, MeOD, δₚₚₘ vs. MeOD): 5.80-5.90 (m, 9H, CH₂C(H)═CH₂); 5.22 (d, J=17 Hz, 9H, CH₂C(H)═CH₂); 5.12 (d, J=10.2 Hz, 9H, CH₂C(H)═CH₂); 3.78 (app d, J=5 Hz, 18H, CH₂—CH═CH₂); 3.36 (t, J=6.7 Hz, 18H, CONHCH₂); 3.26 (t, J=6.7 Hz, 18H, CH₂N₃); 2.46 (app s, 4H, COCH₂CH₂CO); 2.25 (m (br), 48H, CH₂CO); 2.03 (m (br), 48H, C_{q}CH₂); 1.76 (p, J=6.7 Hz, 18H, CH₂CH₂CH₂N₃). ¹³CNMR (150 MHz, MeOD, δₚₚₘ vs. MeOD): 174.29; 174.14; 174.09; 174.03; 172.71; 134.21; 114.94; 57.54; 57.53; 57.52; 48.71; 41.49; 41.44; 36.47; 30.60; 30.53; 30.35; 30.26; 30.18; 29.88; 29.80; 28.38, MALDI-TOF MS m/z calculated for C₁₃₈H₂₂₅N₅₃O₂₆3042.66. found 3043.735.

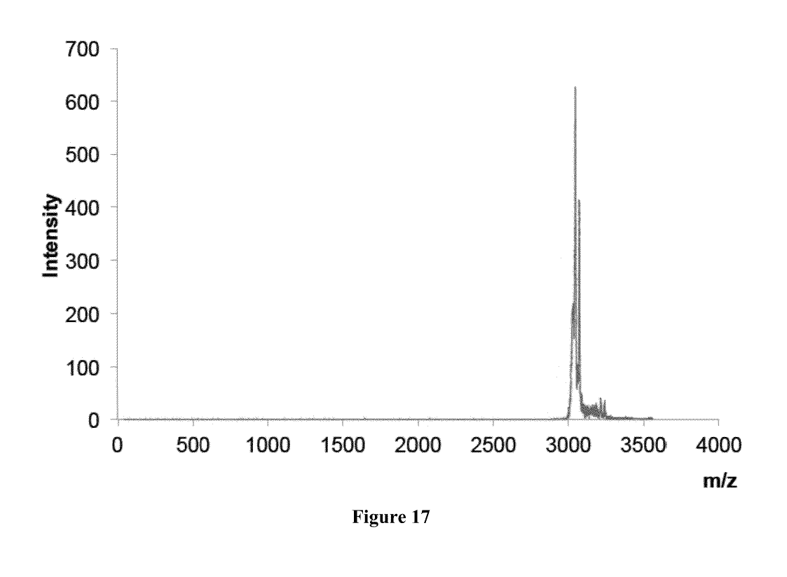

The structure of bifunctional dendrimer 13 was confirmed by ¹HNMR (FIG. 16) and mass spectra (FIG. 17).

### Example 10

### Synthesis of Bifunctional Peptidodendrimer Conjugates

Bifunctional dendrimer 13 was conjugated with HSV-1 envelope glycoprotein peptides gB8 and PgH to form bifunctional peptidodendrimer conjugate 14.

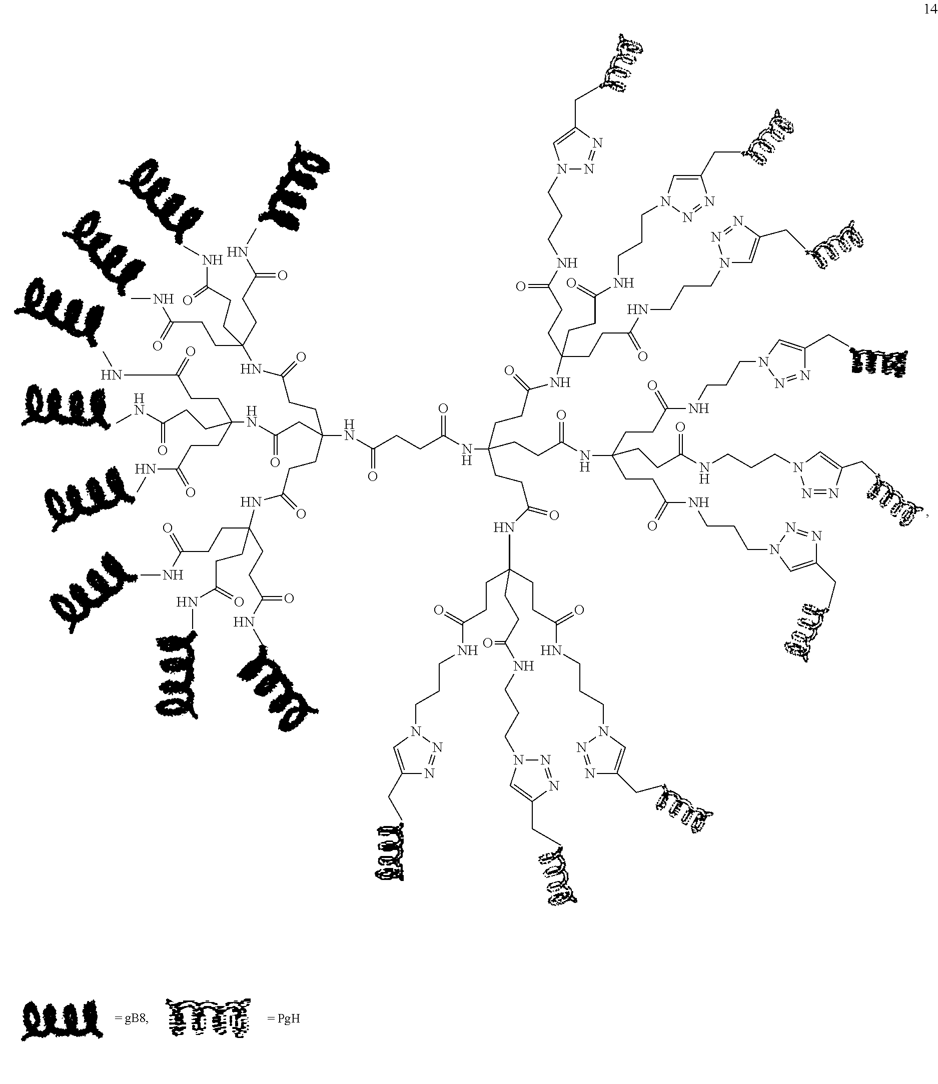

Peptide PgH was attached using standard click-chemistry as described in Example 2 above. Peptide gB8 was coupled by thiolo-ene reaction. Briefly, for the second reaction the peptide gB8 has an extra cysteine residue at the C-terminus. The photoinduced reaction takes place between the thiol of the cysteine residue and the alkene present on the bifunctionalized dendrimer. Coupling of 2-4 equivalents of peptide on the dendrimers was carried out in DMF/H₂O under irradiation for 1 hour at λ365 nm in the presence of 2,2-dimethoxy-2-phenylacetophenone (DPAP) as the initiator.

### Example 11

### Virus Yield Reduction Assay Using a Bifunctional Peptidodendrimer Conjugate

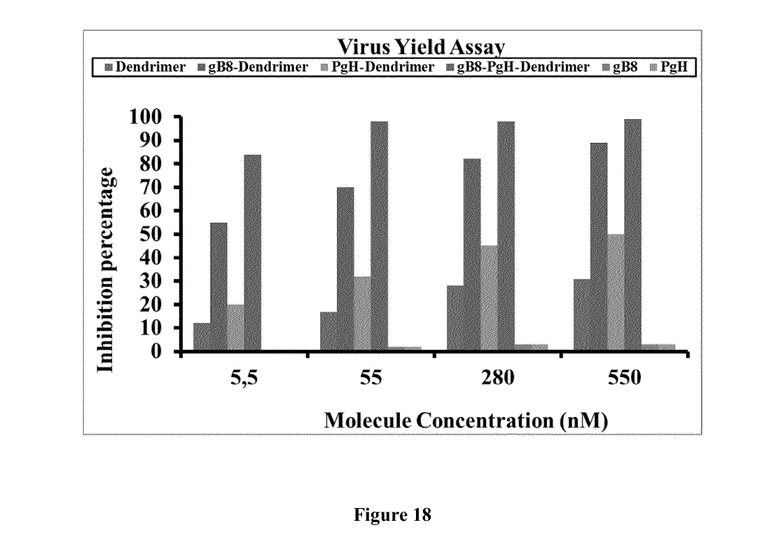

Antiviral activity of bifunctional dendrimer conjugate 14 was evaluated in the same way as described above in Example 4, but using bifunctional peptidodendrimer conjugate 14 (“gB8-PgH-Dendrimer”) in place of the 1:1 mixture of the two monofunctional peptidodendrimer conjugates. See FIG. 18.

### Discussion of Examples 9-11

As shown in FIG. 18, the bifunctional peptidodendrimer conjugate was shown to be significantly more active than either monofunctional peptidodendrimer conjugate alone. It was also found to have significantly improved efficacy compared to the coadministration of the two monofunctional peptidodendrimer conjugates (compare FIG. 18 with FIG. 6), achieving over 80% inhibition at a concentration of only 5.5 nM and nearly 100% inhibition at a concentration of only 55 nM.

Bifunctional dendrimers provide another method for bringing different peptides into close contact with the virus. These results confirm that, as expected from Examples 1-8, bifunctional dendrimers conjugated with two different peptides have considerably higher anti-viral activity relative to the activity achieved with co-administration of monofunctional dendrimers conjugated with the peptides.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A monofunctional peptidodendrimer conjugate comprising: a polyamide dendrimer conjugated with a Herpes Simplex Virus 1 (“HSV-1”) envelope glycoprotein-derived peptide, wherein the peptide is a substituted or unsubstituted peptide selected from the group consisting of gB8, PgH, gC1, g1, and g2.

2. The monofunctional peptidodendrimer conjugate of claim 1, wherein the HSV-1 envelope glycoprotein-derived peptide is a substituted or unsubstituted gB8.

3. The monofunctional peptidodendrimer conjugate of claim 1, wherein the monofunctional peptidodendrimer conjugate has the formula: 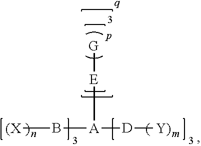
wherein:
A is an amide dendrimer core;
B, D, and E (if present) are each a moiety of formula 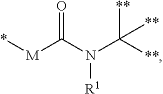
wherein:
*- is the point of attachment to A;
** is the point of attachment to X, Y, or G (if present), with the proviso that when m, n, and p are less than 3, ** can be a point of attachment to hydrogen;
M is an aromatic or aliphatic moiety;
each R¹ is selected from the group consisting of H and C₁₋₃ alkyl; and
each B, D, and E (if present) can be the same or different;
X, Y, and G (if present) are each independently a moiety of formula ***-Q-C(O)—NR²-L-Z—P,
wherein:
***- is the point of attachment to B, D, or E (if present);
Q is optionally present and, if present, is an aromatic or aliphatic moiety;
each R² is selected from the group consisting of H and C₁₋₃ alkyl;
each L is optionally present and, if present, is a linker;
each Z is optionally present and, if present, is a spacer; and
each P is the HSV-1 envelope glycoprotein-derived peptide;
m, n, and p are the same and are each 1, 2, or 3; and
q is 0 or 1.

4. The monofunctional peptidodendrimer conjugate according to claim 3, wherein A is a moiety of formula 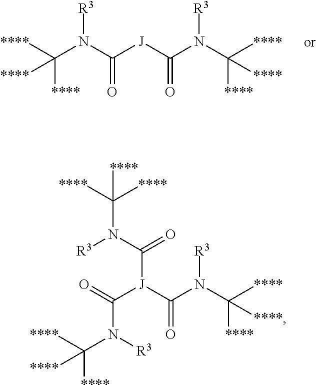
wherein ****- is the point of attachment to B, D, or E (if present); each R³ is selected from the group consisting of H and C₁₋₁₁ alkyl; and J is an aromatic or aliphatic moiety.

5-8. (canceled)

9. The monofunctional peptidodendrimer conjugate according to claim 3, wherein M is:
(i) selected from the group consisting of C₁₋₂₀ alkyl, C₁₋₂₀ alkylene, C₂₋₂₀ alkenyl, C₂₋₂₀ alkenylene, C₂₋₂₀ alkynyl, C₂₋₂₀ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂; or
(ii) a moiety of formula —(CR¹³R¹⁴)ₜ—, wherein t is 0 to 20 and each R¹³ and R¹⁴ are independently selected from the group consisting of H and C₁₋₃ alkyl.

10-11. (canceled)

12. The monofunctional peptidodendrimer conjugate according to claim 3, wherein Q is:
(i) selected from the group consisting of C₁₋₂₀ alkyl, C₁₋₂₀ alkylene, C₂₋₂₀ alkenyl, C₂₋₂₀ alkenylene, C₂₋₂₀ alkynyl, C₂₋₂₀ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂; or
(ii) a moiety of formula —(CR¹⁵R¹⁶)ᵤ—, wherein u is 0 to 20 and each R¹⁵ and R¹⁶ are independently selected from the group consisting of H and C₁₋₃ alkyl.

13. (canceled)

14. The monofunctional peptidodendrimer conjugate according to claim 3, wherein L is a saturated or unsaturated, branched or unbranched, optionally substituted carbon chain of from 1 to about 50 atoms in length, and optionally including from 1 to 25 heteroatoms in the chain.

15. (canceled)

16. The monofunctional peptidodendrimer conjugate according to claim 3, wherein Z a unit formed from a bioconjugation reaction selected from the group consisting of click reactions, Staudinger ligation, Schiff base chemistry, reactions involving the thiol group of a cytosine residue, reactions involving lysine residues, and Diels-Alder reactions.

17. (canceled)

18. The monofunctional peptidodendrimer conjugate according to claim 3, wherein at least one of X, Y, and G (if present) is selected from the group consisting of ***—(CR¹⁵R¹⁶)_{z}—CO—NR²-L-Z—P, ***—(CH₂)₂—CO—NH—Z—P, ***—(CH₂)₂CO—NH—C—P,

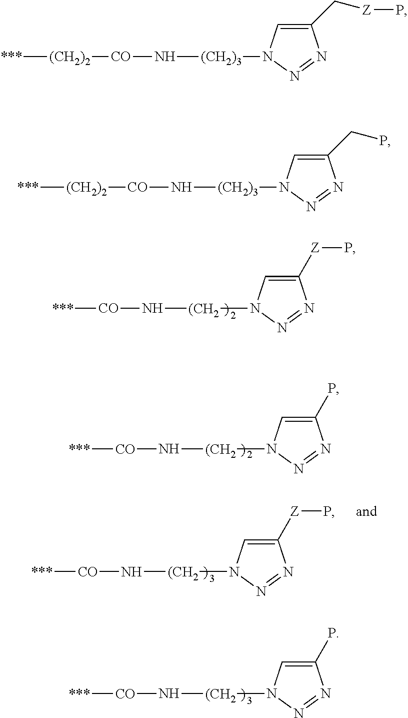

19. A bifunctional peptidodendrimer conjugate comprising: a polyamide dendrimer conjugated with two different HSV-1 envelope glycoprotein-derived peptides.

20. The bifunctional peptidodendrimer conjugate according to claim 19, wherein both HSV-1 envelope glycoprotein-derived peptides are a substituted or unsubstituted peptide selected from the group consisting of the peptides set forth in Table 2 above.

21. The bifunctional peptidodendrimer conjugate according to claim 19, wherein at least one of the HSV-1 envelope glycoprotein-derived peptides is a substituted or unsubstituted peptide selected from the group consisting of gB8, PgH, gC1, g1, and g2.

22-23. (canceled)

24. The bifunctional peptidodendrimer conjugate according to claim 19 having the formula: 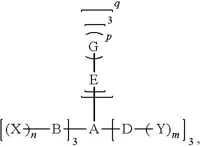
wherein:
A is an amide dendrimer core;
B, D, and E (if present) are each a moiety of formula 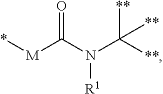
wherein:
*- is the point of attachment to A;
** is the point of attachment to X, Y, or G (if present), with the proviso that when m, n, and p are less than 3, ** can be a point of attachment to hydrogen;
M is an aromatic or aliphatic moiety;
each R¹ is selected from the group consisting of H and C₁₋₃ alkyl; and
each B, D, and E (if present) can be the same or different;
X, Y, and G (if present) are each independently a moiety of formula ***-Q-C(O)—NR²-L-Z—P,
wherein:
***- is the point of attachment to B, D, or E (if present);
Q is optionally present and, if present, is an aromatic or aliphatic moiety;
each R² is selected from the group consisting of H and C₁₋₃ alkyl;
each L is optionally present and, if present, is a linker;
each Z is optionally present and, if present, is a spacer; and
each P is one of the two different HSV-1 envelope glycoprotein-derived peptides;
m, n, and p are the same and are each 1, 2, or 3; and
q is 0 or 1.

25. The bifunctional peptidodendrimer conjugate according to claim 24, wherein A is a moiety of formula 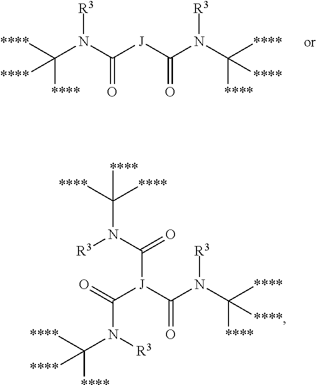
wherein ****- is the point of attachment to B, D, or E (if present); each R³ is selected from the group consisting of H and C₁₋₁₁ alkyl; and J is an aromatic or aliphatic moiety.

26-29. (canceled)

30. The bifunctional peptidodendrimer conjugate according to claim 24, wherein M is:
(i) selected from the group consisting of C₁₋₂₀ alkyl, C₁₋₂₀ alkylene, C₂₋₂₀ alkenyl, C₂₋₂₀ alkenylene, C₂₋₂₀ alkynyl, C₂₋₂₀ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂; or
(ii) a moiety of formula —(CR¹³R¹⁴)t-, wherein t is 0 to 20 and each R¹³ and R¹⁴ are independently selected from the group consisting of H and C₁₋₃ alkyl.

31-32. (canceled)

33. The bifunctional peptidodendrimer conjugate according to claim 24, wherein Q is:
(i) selected from the group consisting of C₁₋₂₀ alkyl, C₁₋₂₀ alkylene, C₂₋₂₀ alkenyl, C₂₋₂₀ alkenylene, C₂₋₂₀ alkynyl, C₂₋₂₀ alkynylene, —C(O)—, —C(O)O—, —O—, —S—, —NH—, —N(R²⁰)—, —NHC(O)—, —N(R²⁰)C(O)—, —Si(R²¹R²²)—, cycloalkyl, cycloalkylene, hydroxyalkyl, hydroxyalkylene, thiol, thioalkyl, alkylthioalkyl, alkoxy, aldehyde, ketone, acid, amine, amide, alcohol, heterocyclyl, aryl, heteroaryl, arylalkyl, and acyl; wherein R²⁰ is selected from the group consisting of C₁₋₂₀ alkyl, C₂₋₂₀ alkenyl, C₂₋₂₀ alkynyl, —OH, —SH, —SC₁₋₂₀ alkyl, —COOH, amine, and aryl; and R²¹ and R²² are independently selected from the group consisting of C₁₋₂₀ alkyl, C₂₋₂₀ alkenyl, —OC₁₋₂₀ alkyl, amine, —OSi(C₁₋₂₀ alkyl)₃, —OSi(C₁₋₂₀ alkyl)₂(C₂₋₂₀ alkenyl), and —OSi(C₁₋₂₀ alkyl)(C₂₋₂₀ alkenyl)₂; or
(ii) a moiety of formula —(CR¹⁵R¹⁶)ᵤ—, wherein u is 0 to 20 and each R¹⁵ and R¹⁶ are independently selected from the group consisting of H and C₁₋₃ alkyl.

34. (canceled)

35. The bifunctional peptidodendrimer conjugate according to claim 24, wherein L is a saturated or unsaturated, branched or unbranched, optionally substituted carbon chain of from 1 to about 50 atoms in length, and optionally including from 1 to 25 heteroatoms in the chain.

36. (canceled)

37. The bifunctional peptidodendrimer conjugate according to claim 24, wherein Z a unit formed from a bioconjugation reaction selected from the group consisting of click reactions, Staudinger ligation, Schiff base chemistry, reactions involving the thiol group of a cytosine residue, reactions involving lysine residues, and Diels-Alder reactions.

38. (canceled)

39. The bifunctional peptidodendrimer conjugate according to claim 24, wherein at least one of X, Y, and G (if present) is selected from the group consisting of ***—(CR¹⁵R¹⁶)₂—CO—NR²-L-Z—P, ***—(CH₂)₂—CO—NH—Z—P, ***—(CH₂)₂CO—NH—C—P,

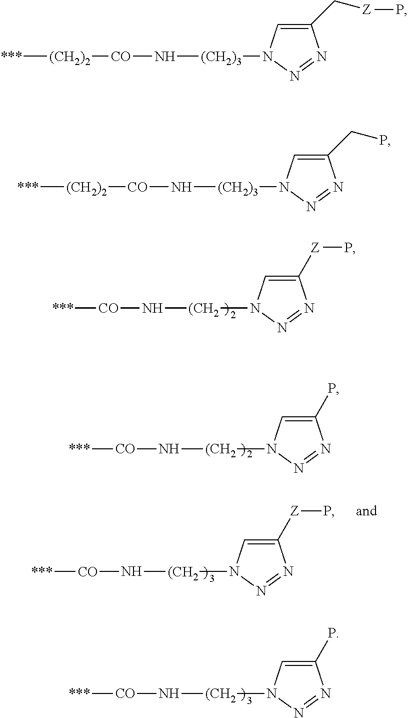

40. A pharmaceutical formulation comprising:
a monofunctional peptidodendrimer conjugate according to claim 1 and
a pharmaceutically acceptable vehicle.

41. A pharmaceutical formulation comprising:
a bifunctional peptidodendrimer conjugate according to claim 19 and
a pharmaceutically acceptable vehicle.

42. A pharmaceutical formulation comprising, in a pharmaceutically acceptable vehicle:
(i) a first monofunctional peptidodendrimer conjugate comprising: a polyamide dendrimer conjugated with a first HSV-1 envelope glycoprotein-derived peptide; and
(ii) a second monofunctional peptidodendrimer conjugate comprising: a polyamide dendrimer conjugated with a second HSV-1 envelope glycoprotein-derived peptide;
wherein the first and second HSV-1 envelope glycoprotein-derived peptides are different.

43. The pharmaceutical formulation according to claim 42, wherein the first HSV-1 envelope glycoprotein-derived peptide and the second HSV-1 envelope glycoprotein-derived peptide are each a substituted or unsubstituted peptide selected from the group consisting of the peptides set forth in Table 2 above.

44. The pharmaceutical formulation according to claim 43, wherein at least one of the first and second HSV-1 envelope glycoprotein-derived peptides is a substituted or unsubstituted peptide selected from the group consisting of gB8, PgH, gC1, g1, and g2.

45-46. (canceled)

47. A method of inhibiting entry of HSV-1 into a host cell, said method comprising:
contacting the host cell, under conditions effective to inhibit entry of HSV-1 into the host cell, with:
(i) a monofunctional peptidodendrimer conjugate comprising: a polyamide dendrimer conjugated with an HSV-1 envelope glycoprotein-derived peptide, wherein the peptide is a substituted or unsubstituted peptide selected from the group consisting of gB8, PgH, gC1, g1, and g2;
(ii) (a) a first monofunctional peptidodendrimer conjugate comprising: a polyamide dendrimer conjugated with a first HSV-1 envelope glycoprotein-derived peptide and (b) a second monofunctional peptidodendrimer conjugate comprising: a polyamide dendrimer conjugated with a second HSV-1 envelope glycoprotein-derived peptide, wherein the first and second HSV-1 envelope glycoprotein-derived peptides are different;
(iii) a bifunctional peptidodendrimer conjugate comprising: a polyamide dendrimer conjugated with two different HSV-1 envelope glycoprotein-derived peptides; or
(iv) a combination thereof.

48. (canceled)

49. A method of treating or preventing HSV-1 infection in a subject, said method comprising:
administering to the subject, under conditions effective to treat or prevent HSV-1 infection:
(i) a monofunctional peptidodendrimer conjugate comprising: a polyamide dendrimer conjugated with an HSV-1 envelope glycoprotein-derived peptide, wherein the peptide is a substituted or unsubstituted peptide selected from the group consisting of gB8, PgH, gC1, g1, and g2;
(ii) (a) a first monofunctional peptidodendrimer conjugate comprising: a polyamide dendrimer conjugated with a first HSV-1 envelope glycoprotein-derived peptide and (b) a second monofunctional peptidodendrimer conjugate comprising: a polyamide dendrimer conjugated with a second HSV-1 envelope glycoprotein-derived peptide, wherein the first and second HSV-1 envelope glycoprotein-derived peptides are different;
(iii) a bifunctional peptidodendrimer conjugate comprising: a polyamide dendrimer conjugated with two different HSV-1 envelope glycoprotein-derived peptides; or
(iv) a combination thereof.

50-51. (canceled)


SEQUENCE LISTING (76 sequences)

SEQ ID NO: 1 - protein, 904 aa, Herpes simplex virus
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 2 - protein, 904 aa, Herpes simplex virus
MRQGAPARGCRWFVVWALLGLTLGVLVASAAPSSPGTPGVAAATQAANGGPATPAPPAPGPAPTGDTKPKKNKKPKNPPPPCPAGDNATVAAGHATLREHLRDIKAKNTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYNEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMEHTEHKAKKKGTSALLSAKVTDMVMRKRRNTNYTQVPNKDSDADEDDL

SEQ ID NO: 3 - protein, 903 aa, Herpes simplex virus
MRQGAARGCRWFVVWALLGLTLGVLVASAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 4 - protein, 903 aa, Herpes simplex virus
MRQGAARGCRWFVVWALLGLTLGVLVASAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQVGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 5 - protein, 904 aa, Herpes simplex virus
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 6 - protein, 904 aa, Herpes simplex virus
MRQGAPARGRRWFVVWALLGLTLGVLVASAAPSSPGTPGVAAATQAANGGPATPAPPAPGAPPTGDPKPKKNKKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYNEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSVMERTEHKAKKKGTSALLSAKVTDMVMRKRRNTNYTQVPNKDGDADEDDL

SEQ ID NO: 7 - protein, 904 aa, Herpes simplex virus
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEESAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 8 - protein, 904 aa, Herpes simplex virus
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAESTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMEHTEHKAKKKGTSALLSAKVTDMVMRKRRNTNYTQVPNKDGDADEDDL

SEQ ID NO: 9 - protein, 904 aa, Artificial
  Consensus sequence of gB glycoprotein
MXQGXXXXGXRWFVVWALLGLTLGVLVASAAPXSPGTPGVAAATQAANGGPATPAPPXXGXXPTGDXKPKKNXKPKXPXPPXPAGDNATVAAGHATLREHLRDIKAXXTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYXEFVLATGDFVYMSPFYGYREGSHTEHTXYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYXANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASXTVGRRVSARMLGDVMAVSTCVPVAADNVIVQNSMRISSRPGACYSRPLVSFRYEDQGPLVEGQXGENNELRLTRDAIEPCTVGHRRYFTFGGGYVYFEEXAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSXMEXTEHKAKKKGTSALLSAKVTDMVMRKRRNTNYTQVPNKDXDADEDDL

SEQ ID NO: 10 - protein, 393 aa, Herpes simplex virus
MGGAAARLGAVILFVVIVGLHGVRGKYALADASLKMADPNRFRGKDLPVLDQLTDPPGVRRVYHIQAGLPNPFQPPSLPITVYRRVERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYTLPLRIPPSACLSPQAYQQGVTVDSIGMLPRFIPENQRTVAVYSLKIAGWHGPRAPYTSTLLPPELPETPNATQPELAPEDPEDSALLEDPVGTVAPQIPPNWHIPSIQDAATPYHPPATPNNMGLIAGAVGGSLLAALVICGIVYWMRRRTRKAPKRIRLPHIREDDQPSSHQPLFY

SEQ ID NO: 11 - protein, 394 aa, Herpes simplex virus
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 12 - protein, 394 aa, Herpes simplex virus
MGGTAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 13 - protein, 394 aa, Herpes simplex virus
MGGAAARLGAVILFVVIVGLHGVRGKYALADASLKMADPNRFRGKDLPVPDRLTDPPGVRRVYHIQAGLPDPFQPPSLPITVYYAVLERACRSVLLNAPSEAPQIVRGGSEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRIVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKGPKRIRLPHIREDDQPSSHQPLFY

SEQ ID NO: 14 - protein, 394 aa, Artificial
  Consensus sequence of gD glycoprotein
MGGXAARLGAVILFVVIVGLHGVRXKYALXDASLKMADPNRFRGKDLPVXDXLTDPPGVRRVYHIQAGLPXPFQPPSLPITVYXXXXERACRSVLLNAPSEAPQIVRGXSEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYXLPLRIPPSACLSPQAYQQGVTVDSIGMLPRFIPENQRXVAVYSLKIAGWHGPXAPYTSTLLPPELXETPNATQPELAPEDPEDSALLEDPVGTVAPQIPPNWHIPSIQDAATPYHPPATPNNMGLIAGAVGGSLLAALVICGIVYWMXRXTXKXPKRIRLPHIREDDQPSSHQPLFY

SEQ ID NO: 15 - protein, 838 aa, Herpes simplex virus
MGNGLWFVGVIILGAAWGQVHDWTEQTDPWFLDGLGMDRMYWRDTNTGRLWLPNTPDPQKPPRGFLAPPDELNLTTASLPLLRWYEERFCFVLVTTAEFPRDPGQLLYIPKTYLLGRPPNASLPAPTTVEPTAQPPPAVAPLKGLLHNPTASVLLRSRAWVTFSAVPDPEALTFPRGDNVATASHPSGPRDTPPPRPPVGARRHPTTELDITHLHNASTTWLATRGLLRSPGRYVYFSPSASTWPVGIWTTGELVLGCDAALVRARYGREFMGLVISMHDSPPAEVMVVPAGQTLDRVGDPADENPPGALPGPPGGPRYRVFVLGSLTRADNGSALDALRRVGGYPEEGTNYAQFLSRAYAEFFSGDAGAEQGPRPPLFWRLTGLLATSGFAFVNAAHANGAVCLSDLLGFLAHSRALAGLAARGAAGCAADSVFFNVSVLDPTARLQLEARLQHLVAEILEREQSLALHALGYQLAFVLDSPSAYDAVAPSAAHLIDALYAEFLGGRVLTTPVVHRALFYASAVLRQPFLAGVPSAVQRERARRSLLIASALCTSDVAAATNADLRTALARADHQKTLFWLPDHFSPCAASLRFDLDESVFILDALAQATRSETPVEVLAQQTHGLASTLTRWAHYNALIRAFVPEASHRCGGQSANVEPRILVPITHNASYVVTHSPLPRGIGYKLTGVDVRRPLFLTYLTATCEGSTRDIESKRLVRTQNQRDLGLVGAVFMRYTPAGEVMSVLLVDTDNTQQQIAAGPTEGAPSVFSSDVPSTALLLFPNGTVIHLLAFDTQPVAAIAPGFLAASALGVVMITAALAGILKVLRTSVPFFWRRE

SEQ ID NO: 16 - protein, 838 aa, Herpes simplex virus
MGNGLWFVGVIILGAAWGQVHDWTEQTDPWFLDGLGMDRMYWRDTNTGRLWLPNTPDPQKPPRGFLAPPDELNLTTASLPLLRWYEERFCFVLVTTAEFPRDPGQLLYIPKTYLLGRPPNASLPAPTTVEPTAQPPPAVAPLKGLLHNPTASVLLRSRAWVTFSAVPDPEALTFPRGDNVATASHPSGPRDTPPPRPPVGARRHPTTELDITHLHNASTTWLATRGLLRSPGRYVYFSPSASTWPVGIWTTGELVLGCDAALVRARYGREFMGLVISMHDSPPAEVMVVPAGQTLDRVGDPADENPPGALPGPPGGPRYRVFVLGSLTRADNGSALDALRRVGGYPEEGTNYAQFLSRAYAEFFSGDAGAEQGPRPPLFWRLTGLLATSGFAFVNAAHANGAVCLSDLLGFLAHSRALAGLAARGAAGCAADSVFFNVSVLDPTARLQLEARLQHLVAEILEREQSLALHALGYQLAFVLDSPSAYDAVAPSAAHLIDALYAEFLGGRVVTTPVVHRALFYASAVLRQPFLAGVPSAVQRERARRSLLIASALCTSDVAAATNADLRTALARADHQKTLFWLPDHFSPCAASLRFDLDESVFILDALAQATRSETPVEVLAQQTHGLASTLTRWAHYNALIRAFVPEASHRCGGQSANVEPRILVPITHNASYVVTHSPLPRGIGYKLTGVDVRRPLFLTYLTATCEGSTRDIESKRLVRTQNQRDLGLVGAVFMRYTPAGEVMSVLLVDTDNTQQQIAAGPTEGAPSVFSSDVPSTALLLFPNGTVIHLLAFDTQPVAAIAPGFLAASALGVVMITAALAGILKVLRTSVPFFWRRE

SEQ ID NO: 17 - protein, 838 aa, Herpes simplex virus
MGNGLWFVGVIILGVAWGQVHDWTEQTDPWFLDGLGMDRMYWRDTNTGRLWLPNTPDPQKPPRGFLAPPDELNLTTASLPLLRWYEERFCFVLVTTAEFPRDPGQLLYIPKTYLLGRPPNASLPAPTTVEPTAQPPPSVAPLKGLLHNPAASVLLRSRAWVTFSAVPDPEALTFPRGDNVATASHPSGPRDTPPPRPPVGARRHPTTELDITHLHNASTTWLATRGLLRSPGRYVYFSPSASTWPVGIWTTGELVLGCDAALVRARYGREFMGLVISMHDSPPVEVMVVPAGQTLDRVGDPADENPPGALPGPPGGPRYRVFVLGSLTRADNGSALDALRRVGGYPEEGTNYAQFLSRAYAEFFSGDAGAEQGPRPPLFWRLTGLLATSGFAFVNAAHANGAVCLSDLLGFLAHSRALAGLAARGAAGCAADSVFFNVSVLDPTARLQLEARLQHLVAEILEREQSLALHALGYQLAFVLDSPSAYDAVAPSAAHLIDALYAEFLGGRVLTTPVVHRALFYASAVLRQPFLAGVPSAVQRERARRSLLIASALCTSDVAAATNADLRTALARADHQKTLFWLPDHFSPCAASLRFDLDESVFILDALAQATRSETPVEVLAQQTHGLASTLTRWAHYNALIRAFVPEASHRCGGQSANVEPRILVPITHNASYVVTHSPLPRGIGYKLTGVDVRRPLFLTYLTATCEGSTRDIESKRLVRTQNQRDLGLVGAVFMRYTPAGEVMSVLLVDTDNTQQQIAAGPTEGAPSVFSSDVPSTALLLFPNGTVIHLLAFDTQPVAAIAPGFLAASALGVVMITAALAGILKVLRTSVPFFWRRE

SEQ ID NO: 18 - protein, 838 aa, Artificial
  Consensus sequence of gH glycoprotein
MGNGLWFVGVIILGXAWGQVHDWTEQTDPWFLDGLGMDRMYWRDTNTGRLWLPNTPDPQKPPRGFLAPPDELNLTTASLPLLRWYEERFCFVLVTTAEFPRDPGQLLYIPKTYLLGRPPNASLPAPTTVEPTAQPPPXVAPLKGLLHNPXASVLLRSRAWVTFSAVPDPEALTFPRGDNVATASHPSGPRDTPPPRPPVGARRHPTTELDITHLHNASTTWLATRGLLRSPGRYVYFSPSASTWPVGIWTTGELVLGCDAALVRARYGREFMGLVISMHDSPPXEVMVVPAGQTLDRVGDPADENPPGALPGPPGGPRYRVFVLGSLTRADNGSALDALRRVGGYPEEGTNYAQFLSRAYAEFFSGDAGAEQGPRPPLFWRLTGLLATSGFAFVNAAHANGAVCLSDLLGFLAHSRALAGLAARGAAGCAADSVFFNVSVLDPTARLQLEARLQHLVAEILEREQSLALHALGYQLAFVLDSPSAYDAVAPSAAHLIDALYAEFLGGRVXTTPVVHRALFYASAVLRQPFLAGVPSAVQRERARRSLLIASALCTSDVAAATNADLRTALARADHQKTLFWLPDHFSPCAASLRFDLDESVFILDALAQATRSETPVEVLAQQTHGLASTLTRWAHYNALIRAFVPEASHRCGGQSANVEPRILVPITHNASYVVTHSPLPRGIGYKLTGVDVRRPLFLTYLTATCEGSTRDIESKRLVRTQNQRDLGLVGAVFMRYTPAGEVMSVLLVDTDNTQQQIAAGPTEGAPSVFSSDVPSTALLLFPNGTVIHLLAFDTQPVAAIAPGFLAASALGVVMITAALAGILKVLRTSVPFFWRRE

SEQ ID NO: 19 - protein, 511 aa, Herpes simplex virus
MAPGRVGLAVVLWGLLWLGAGVAGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 20 - protein, 166 aa, Herpes simplex virus
QLLYISKTYLLGRPPNASLPAPITVEPTAQPPPAVAPLKGLLHNPTASVLLRSRAWVTFSAVPDPEALTFPRGDNVATASHPSGPRDTPPPRPPVGARRHPTTELDITHLHNASTTWLATRGLLRSPGRYVYFSPSASTCPVGIWTTGELVLGCDSAGRARYGREF

SEQ ID NO: 21 - protein, 224 aa, Herpes simplex virus
MGFVCLFGLVVMGAWGAWGGSQATEYVLRSVIAKEVGDILRVPCMRTPADDVSWRYEAPSVIDYARIDGIFLRYHCPGLDTFLWDRHAQRAYLVNPFLFAAGFLEDLSHSVFPADTQETTTRRALYKEIRDALGSRKQAVSHAPVRAGCVNFDYSRTRRCVGRRDLRPANTTSTWEPPVSSDDEASSQSKPLATQPPVLALSNAPPRRVSPTRGRRRHTRLRRN

SEQ ID NO: 22 - protein, 224 aa, Herpes simplex virus
MGILGWVGLIAVGVLCVRGGLPSTEYVIRSRVAREVGDILKVPCVPLPSDDLDWRYETPSAINYALIDGIFLRYHCPGLDTVLWDRHAQKAYWVNPFLFVAGFLEDLSYPAFPANTQETETRLALYKEIRQALDSRKQAASHTPVKAGCVNFDYSRTRRCVGRQDLGPTNGTSGRTPVLPPDDEAGLQPKPLTTPPPIIATSDPTPRRDAATKSRRRRPHSRRL

SEQ ID NO: 23 - protein, 21 aa, Artificial
  HSV-1 envelope glycoprotein-derived peptide
FARLQFTYNHIQRHVRDMEGR

SEQ ID NO: 24 - protein, 19 aa, Artificial
  HSV-1 envelope glycoprotein-derived peptide
VTVSQVWFGHRYSQFMGIF

SEQ ID NO: 25 - protein, 19 aa, Artificial
  HSV-1 envelope glycoprotein-derived peptide
FVLATGDFVYMSPFYGYRE

SEQ ID NO: 26 - protein, 19 aa, Artificial
  HSV-1 envelope glycoprotein-derived peptide
YGGSFRFSSDAISTTFTTN

SEQ ID NO: 27 - protein, 19 aa, Artificial
  HSV-1 envelope glycoprotein-derived peptide
YYLANGGFLIAYQPLLSNT

SEQ ID NO: 28 - protein, 24 aa, Artificial
  HSV-1 envelope glycoprotein-derived peptide
SVERIKTTSSIEFARLQFTYNHIQ

SEQ ID NO: 29 - protein, 19 aa, Artificial
  HSV-1 envelope glycoprotein-derived peptide
YAYSHQLSRADITTVSTFI

SEQ ID NO: 30 - protein, 45 aa, Artificial
  HSV-1 envelope glycoprotein-derived peptide
SIEFARLQFTYNHIQRHVNDMLGRVAIAWCELQNHELTLWNEARK

SEQ ID NO: 31 - protein, 24 aa, Artificial
  HSV-1 envelope glycoprotein-derived peptide
SIEFARLQFTYNHIQRHVNDMLGR

SEQ ID NO: 32 - protein, 21 aa, Artificial
  HSV-1 envelope glycoprotein-derived peptide
VAIAWCELQNHELTLWNEARK

SEQ ID NO: 33 - protein, 21 aa, Artificial
  HSV-1 envelope glycoprotein-derived peptide
FARLQFTYNHIQRHVNDMLGR

SEQ ID NO: 34 - protein, 14 aa, Artificial
  HSV-1 envelope glycoprotein-derived peptide
YNHIQRHVNDMLGR

SEQ ID NO: 35 - protein, 21 aa, Artificial
  HSV-1 envelope glycoprotein-derived peptide
YNHIQRHVNDMLGRVAIAWCE

SEQ ID NO: 36 - protein, 21 aa, Artificial
  HSV-1 envelope glycoprotein-derived peptide
YNHIQRHVNDMLGRVKKAWEE

SEQ ID NO: 37 - protein, 28 aa, Artificial
  HSV-1 envelope glycoprotein-derived peptide
FARLQFTYNHIQRHVNDMLGRVAIAWCE

SEQ ID NO: 38 - protein, 28 aa, Artificial
  HSV-1 envelope glycoprotein-derived peptide
FARLQFTYNHIQRHVNDMLGRVKKAWEE

SEQ ID NO: 39 - protein, 36 aa, Artificial
  HSV-1 envelope glycoprotein-derived peptide
SIEFARLQFTYNHIQRHVNDMLGRVAIAWCELQNHE

SEQ ID NO: 40 - protein, 18 aa, Artificial
  HSV-1 envelope glycoprotein-derived peptide
IVTTTWAGSTYAAITNQY

SEQ ID NO: 41 - protein, 24 aa, Artificial
  HSV-1 envelope glycoprotein-derived peptide
AGGRVTTVSLAEFAALQFTHDHTR

SEQ ID NO: 42 - protein, 15 aa, Artificial
  HSV-1 envelope glycoprotein-derived peptide
ANHKRYFRFGADYVY

SEQ ID NO: 43 - protein, 15 aa, Artificial
  HSV-1 envelope glycoprotein-derived peptide
GSRVQIRCRFRNSTR

SEQ ID NO: 44 - protein, 15 aa, Artificial
  HSV-1 envelope glycoprotein-derived peptide
GSRVQITCRFRNSTR

SEQ ID NO: 45 - protein, 15 aa, Artificial
  HSV-1 envelope glycoprotein-derived peptide
GSRVQIRCTFRNSTR

SEQ ID NO: 46 - protein, 15 aa, Artificial
  HSV-1 envelope glycoprotein-derived peptide
GSRVQIRCRFTNSTR

SEQ ID NO: 47 - protein, 15 aa, Artificial
  HSV-1 envelope glycoprotein-derived peptide
GSRVQITCTFTNSTR

SEQ ID NO: 48 - protein, 15 aa, Artificial
  HSV-1 envelope glycoprotein-derived peptide
GSTVQIRCRFRNSTT

SEQ ID NO: 49 - protein, 15 aa, Artificial
  HSV-1 envelope glycoprotein-derived peptide
GSRTQIRCRFRNSTR

SEQ ID NO: 50 - protein, 15 aa, Artificial
  HSV-1 envelope glycoprotein-derived peptide
GSRVQTRCRFRNSTR

SEQ ID NO: 51 - protein, 15 aa, Artificial
  HSV-1 envelope glycoprotein-derived peptide
GSRVQIRCRTRNSTR

SEQ ID NO: 52 - protein, 15 aa, Artificial
  HSV-1 envelope glycoprotein-derived peptide
GSRTQTRCRTRNSTR

SEQ ID NO: 53 - protein, 20 aa, Artificial
  HSV-1 envelope glycoprotein-derived peptide
AAHLIDALYAEFLGGRVLTT

SEQ ID NO: 54 - protein, 21 aa, Artificial
  HSV-1 envelope glycoprotein-derived peptide
HGLASTLTRWAHYNALIRAFX

SEQ ID NO: 55 - protein, 43 aa, Artificial
  HSV-1 envelope glycoprotein-derived peptide
TWLATRGLLRSPGRYVYFSPSASTWPVGIWTTGELVLGCDAAL

SEQ ID NO: 56 - protein, 40 aa, Artificial
  HSV-1 envelope glycoprotein-derived peptide
RLTGLLATSGFAFVNAAHANGAVCLSDLLGFLAHSRALAG

SEQ ID NO: 57 - protein, 45 aa, Artificial
  HSV-1 envelope glycoprotein-derived peptide
AAHLIDALYAEFLGGRVLTTPVVHRALFYASAVLRQPFLAGVPSA

SEQ ID NO: 58 - protein, 19 aa, Artificial
  HSV-1 envelope glycoprotein-derived peptide
GLASTLTRWAHYNALIRAF

SEQ ID NO: 59 - protein, 25 aa, Artificial
  HSV-1 envelope glycoprotein-derived peptide
ASLRFDLDESVFILDALAQATRSET

SEQ ID NO: 60 - protein, 25 aa, Artificial
  HSV-1 envelope glycoprotein-derived peptide
VFILDALAQATRSETPVEVLAQQTH

SEQ ID NO: 61 - protein, 12 aa, Artificial
  HSV-1 envelope glycoprotein-derived peptide
LRSRTKIIRIRH

SEQ ID NO: 62 - protein, 10 aa, Artificial
  HSV-1 envelope glycoprotein-derived peptide
RSRTKIIRIR

SEQ ID NO: 63 - protein, 12 aa, Artificial
  HSV-1 envelope glycoprotein-derived peptide
MPRRRRIRRRQK

SEQ ID NO: 64 - protein, 10 aa, Artificial
  HSV-1 envelope glycoprotein-derived peptide
RRRRIRRRQK

SEQ ID NO: 65 - protein, 12 aa, Artificial
  HSV-1 envelope glycoprotein-derived peptide
APRRRRIRRRQK

SEQ ID NO: 66 - protein, 12 aa, Artificial
  HSV-1 envelope glycoprotein-derived peptide
MARRRRIRRRQK

SEQ ID NO: 67 - protein, 12 aa, Artificial
  HSV-1 envelope glycoprotein-derived peptide
MPARRRIRRRQK

SEQ ID NO: 68 - protein, 12 aa, Artificial
  HSV-1 envelope glycoprotein-derived peptide
MPRARRIRRRQK

SEQ ID NO: 69 - protein, 12 aa, Artificial
  HSV-1 envelope glycoprotein-derived peptide
MPRRARIRRRQK

SEQ ID NO: 70 - protein, 12 aa, Artificial
  HSV-1 envelope glycoprotein-derived peptide
MPRRRAIRRRQK

SEQ ID NO: 71 - protein, 12 aa, Artificial
  HSV-1 envelope glycoprotein-derived peptide
MPRRRRARRRQK

SEQ ID NO: 72 - protein, 12 aa, Artificial
  HSV-1 envelope glycoprotein-derived peptide
MPRRRRIARRQK

SEQ ID NO: 73 - protein, 12 aa, Artificial
  HSV-1 envelope glycoprotein-derived peptide
MPRRRRIRARQK

SEQ ID NO: 74 - protein, 12 aa, Artificial
  HSV-1 envelope glycoprotein-derived peptide
MPRRRRIRRAQK

SEQ ID NO: 75 - protein, 12 aa, Artificial
  HSV-1 envelope glycoprotein-derived peptide
MPRRRRIRRRAK

SEQ ID NO: 76 - protein, 12 aa, Artificial
  HSV-1 envelope glycoprotein-derived peptide
MPRRRRIRRRQA